(19) United States
(12) Patent Application Publication
(10) Pub. No.: US 2025/0205204 A1
(43) Pub. Date: Jun. 26, 2025

(54) Transcription Factor Brn2 Inhibitory Compounds for Use as Therapeutics

(71) Applicant: The University of British Columbia, Vancouver (CA)

(72) Inventors: Daksh Thaper, Vancouver (CA); Amina Zoubeidi, Vancouver (CA); Ravi Shashi Nayana Munuganti, Vancouver (CA)

(21) Appl. No.: 18/684,637

(22) PCT Filed: Aug. 23, 2022
(86) PCT No.: PCT/CA2022/051275

§ 371 Date: Feb. 16, 2024

Related U.S. Application Data
(60) Provisional application No. 63/236,200, filed on Aug. 23, 2021.

(51) Int. Cl. A61K 31/428; C07D 221/04; C07D 247/02; C07D 277/82; C07D 417/12

(52) U.S. Cl. CPC A61K 31/428; C07D 221/04; C07D 247/02; C07D 277/82; C07D 417/12

(57) ABSTRACT

This invention provides compound having a structure of Formula (I) or (II): (I, II) and uses of such compounds for treatment of various indications, including prostate cancer as well as methods of treatment involving such compounds are also provide. Furthermore, the compounds described herein may be used for the treatment of BRN2 expressing cancers. The cancer selected from prostate cancer; lung cancer; bladder cancer; sarcoma; glioma; and melanoma.

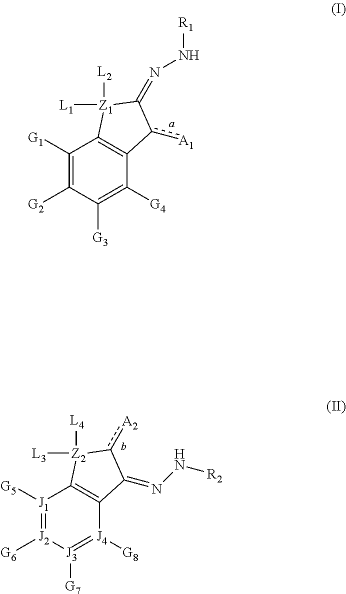

### CROSS REFERENCE TO RELATED APPLICATIONS

This application claims the benefit of U.S. Provisional Patent Application Ser. No. 63/236,200 filed 23 Aug. 2021 entitled “TRANSCRIPTION FACTOR BRN2 INHIBITORY COMPOUNDS FOR USE AS THERAPEUTICS”.

### TECHNICAL FIELD

This invention relates to therapeutics, their uses and methods for the treatment of various indications, including various cancers. In particular, the invention relates to therapies and methods of treatment for cancers such as prostate cancer.

### BACKGROUND

Progression from primary prostate cancer to advanced metastatic disease is heavily dependent on the androgen receptor (AR), which fuels tumor survival. In men whose treatments for localized prostate tumors have failed, or in those who present with metastatic disease, androgen deprivation therapies (ADT) are used to deplete circulating androgens to abrogate AR signaling and prevent disease progression. Eventually, however, prostate cancer recurs after first-line ADT as castration-resistant prostate cancer (CRPC). Despite low levels of serum androgens in men with CRPC, reactivation of the AR occurs; thus, it remains central to tumor cell survival, proliferation, and metastatic spread. Targeting the AR is a cornerstone therapeutic intervention in patients with CRPC, and AR pathway inhibitors (API) that further prevent AR activation, such as enzalutamide (ENZ), have become mainstays in the prostate cancer treatment landscape. Despite its being a potent API, the treatment benefits of ENZ are short-lived in patients with CRPC and resistance rapidly occurs.

ENZ-resistant (ENZ^{R}) CRPC represents a significant clinical challenge not only due to the lack of third-line treatment options to prevent AR-driven tumor progression but also because it can be a precursor to rapidly progressing and lethal neuroendocrine prostate cancer (NEPC). Although NEPC can rarely arise de novo, it is increasingly defined as a variant of highly API-resistant CRPC. Aside from the unique small-cell morphology and positive staining for neuroendocrine (NE) markers that characterize NEPC, it is often distinguished from prostatic adenocarcinoma by reduced AR expression or activity. Clinical presentation of NEPC reflects this shift away from reliance on the AR, as patients typically present with low circulating levels of PSA despite high metastatic burden in soft tissues, and are refractory to APIs. Importantly, it has been reported that under the strong selective pressure of potent APIs like ENZ, these “non-AR-driven” prostate cancers, which include NEPC, may constitute up to 25% of advanced, drug-resistant CRPC cases. Not surprisingly, therefore, the incidence of NEPC has significantly increased in recent years, coinciding with the widespread clinical use of APIs.

A number of molecular mechanisms likely facilitate the progression of CRPC to NEPC. These include loss of tumor suppressors, such as RB1 and p53, amplification of MYCN, mitotic deregulation through AURKA and PEG10, epigenetic controls such as REST and EZH2, and splicing factors like SSRM4.

Importantly, the AR plays a crucial, albeit still mechanistically unclear, role in NEPC. Reports over many years have highlighted how ADT or loss of AR promotes the NE differentiation of prostate cancer cells; as such, many genes associated with an NE phenotype, including ARG2, HASH1, and REST are controlled by the AR. Although this evidence underscores an inverse correlation between AR expression and/or activity and molecular events leading to NEPC, the mechanisms by which the AR directly influences the induction of an NEPC phenotype from CRPC under the selective pressure of APIs such as ENZ remain elusive.

Answering such questions requires a model of API-resistant CRPC that recapitulates the transdifferentiation of adenocarcinoma to NEPC that occurs in patients. Herein, we present an in vivo—derived model of ENZ^{R}, which, different from others, underscores the emergence of tumors with heterogeneous mechanisms of resistance to ENZ over multiple transplanted generations. These include the natural acquisition of known AR mutations found in ENZ^{R} patients and the transdifferentiation of NEPC-like tumors through an AR⁺ state, without the manipulation of oncogenes typically used to establish NEPC in murine prostate cancer models. Bishop et al. (Cancer Discovery (2017) 7(1):54-71) show that a master regulator of neuronal differentiation, the POU-domain transcription factor BRN2 (encoded by POU3F2), is directly transcriptionally repressed by the AR, is required for the expression of terminal NE markers and aggressive growth of ENZ R CRPC, and is highly expressed in human NEPC and metastatic CRPC with low circulating PSA. Beyond suppressing BRN2 expression and activity, Bishop et al. show that the AR inhibits BRN2 regulation of SOX2, another transcription factor associated with NEPC, suggesting that relief of AR-mediated suppression of BRN2 is a consequence of ENZ treatment in CRPC that may facilitate the progression of NEPC, especially in men with “non-AR driven” disease.

### SUMMARY

This invention is based in part on the fortuitous discovery that compounds described herein modulate BRN2 activity. Specifically, some compounds identified herein, also show inhibition of BRN2 and a further subset show specificity for BRN2 expressing cells.

In accordance with a first aspect, there is provided a compound having the structure of Formula I or Formula II:

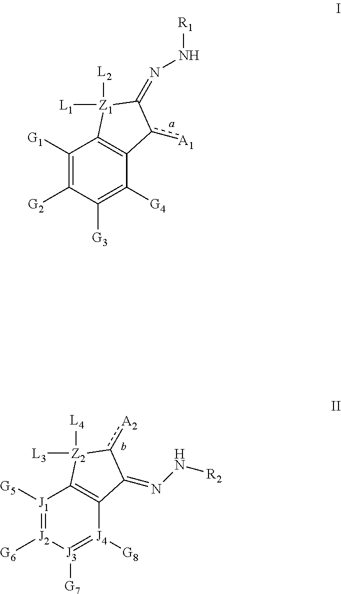

wherein, [symbol] at a may be either a double bond or two single bonds; [symbol] at b may be either a double bond or two single bonds; Z₁ may be selected from C and N; Z₂ may be selected from C, O and N; L₁ may be selected from H, CH₃, CH₂CH₃ and F or is absent when Z₁ is N; L₂ may be selected from H, CH₃, CH₂CH₃ and F; L₃ may be selected from H, CH₃, CH₂CH₃ and F or is absent when Z₂ is N; L₄ may be selected from H, CH₃, CH₂CH₃ and F; A₁ may be O when [symbol] at a is a double bond or A₁ may be two H when at a is two single bonds; A₂ may be O when [symbol] at b is a double bond or A₂ may be two CH₃ groups or two H when [symbol] at b is two single bonds; G₁ may be selected from H, OH, CH₃, CF₃,

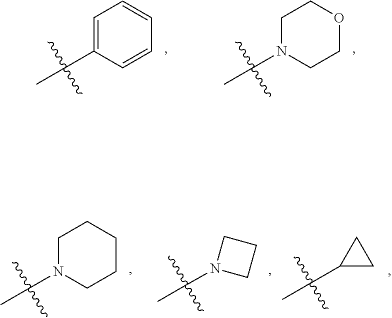

CN, OCH₃, OCHCH₃(CH₃), CH₂CH₃, CH(CH₃)CH₃, N(CH₃)CH₃, N(CH₂CH₃)CH₂CH₃, NH₂, tBuO₂C, F, Cl and Br; G₂ may be selected from H, OH, CH₃, CF₃,

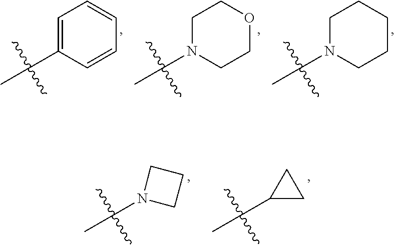

CN, OCH₃, OCHCH₃(CH₃), CH₂CH₃, CH(CH₃)CH₃, N(CH₃)CH₃, N(CH₂CH₃)CH₂CH₃, NH₂, tBuO₂C, F, Cl and Br; G₃ may be selected from H, OH, CH₃, CF₃,

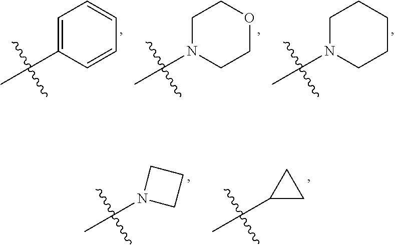

CN, OCH₃, OCHCH₃(CH₃), CH₂CH₃, CH(CH₃)CH₃, N(CH₃)CH₃, N(CH₂CH₃)CH₂CH₃, NH₂, tBuO₂C, F, Cl and Br; G₄ may be selected from H, OH, CH₃,

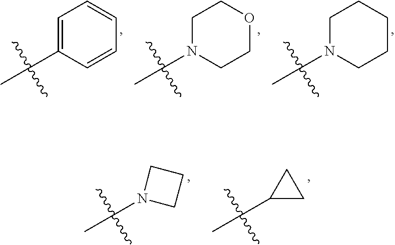

CN, OCH₃, OCHCH₃(CH₃), CH₂CH₃, CH(CH₃)CH₃, N(CH₃)CH₃, N(CH₂CH₃)CH₂CH₃, NH₂, tBuO₂C, F, Cl and Br; G₅ may be selected from H, CH₃, OCH₃, CH₂CH₃, CF₃, F, Cl and Br or is absent when J₁ is N; G₆ may be selected from H, CH₃, OCH₃, CH₂CH₃, CF₃, F, Cl and Br or is absent when J₂ is N; G₇ may be selected from H, CH₃, OCH₃, CH₂CH₃, CF₃ and F or is absent when J₃ is N; G₈ may be selected from H, CH₃, OCH₃, CH₂CH₃ and F or is absent when J₄ is N; J₁ may be selected from C and N; J₂ may be selected from C and N; J₃ may be selected from C and N; J₄ may be selected from C and N; R₁ may be selected from

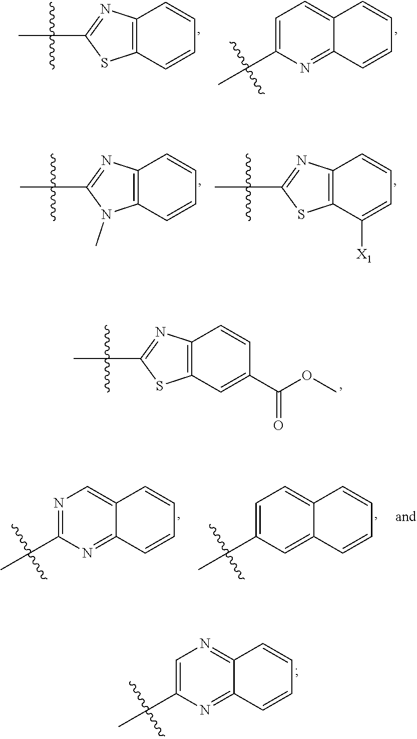

R₂ may be selected from

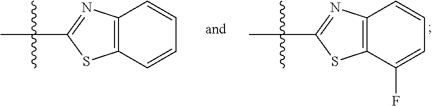 [SMILES: *c1nc2ccccc2s1 ; *c1nc2cccc(F)c2s1]

X₁ may be selected from F, Cl and Br; and provided that the compound is not

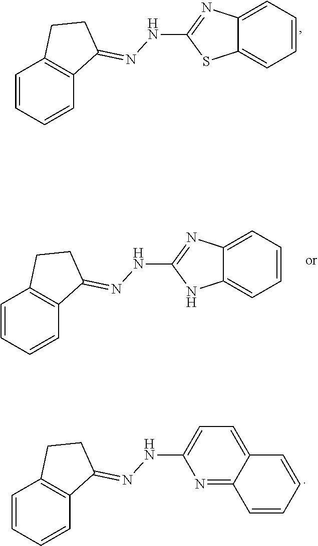 [SMILES: c1ccc2c(c1)CC/C2=N\Nc1nc2ccccc2s1 ; c1ccc2c(c1)CC/C2=N\Nc1nc2ccccc2[nH]1 ; c1ccc2c(c1)CC/C2=N\Nc1ccc2ccccc2n1]

In accordance with a further aspect, there is provided a compound having the structure of Formula I or Formula II:

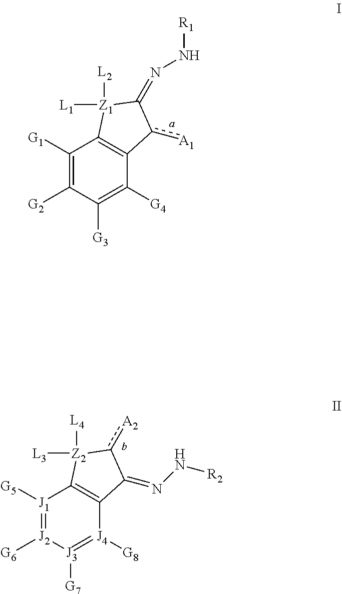

wherein, [symbol] at a may be either a double bond or two single bonds; [symbol] at b may be either a double bond or two single bonds; Z₁ may be C; Z₂ may be selected from C and O; L₁ may be selected from H, CH₃, CH₂CH₃ and F; L₂ may be selected from H, CH₃, CH₂CH₃ and F; L₃ may be selected from H, CH₃, CH₂CH₃ and F or is absent when Z₂ is N; L₄ may be selected from H, CH₃, CH₂CH₃ and F; A₁ may be O when [symbol] at a is a double bond or A₁ is two H when [symbol] at a is two single bonds; A₂ may be O when [symbol] at b is a double bond or A₂ is two CH₃ groups or two H when [symbol] at b is two single bonds; G₁ may be selected from H, OH, CH₃, CF₃,

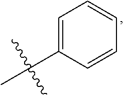 [SMILES: *c1ccccc1]

CN, OCH₃, OCHCH₃(CH₃), N(CH₂CH₃)CH₂CH₃, F, Cl and Br; G₂ may be selected from H, OH, CH₃, CF₃,

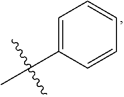 [SMILES: *c1ccccc1]

CN, OCH₃, OCHCH₃(CH₃), N(CH₂CH₃)CH₂CH₃, F, Cl and Br; G₃ may be selected from H, OH, CH₃, CF₃,

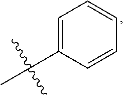 [SMILES: *c1ccccc1]

CN, OCH₃, OCHCH₃(CH₃), N(CH₂CH₃)CH₂CH₃, F, Cl and Br; G₄ may be selected from H, OH, CH₃, CF₃,

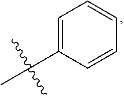 [SMILES: *c1ccccc1]

CN, OCH₃, OCHCH₃(CH₃), N(CH₂CH₃)CH₂CH₃, F, Cl and Br; G₅ may be selected from H, CH₃, OCH₃, CH₂CH₃, CF₃, F, Cl and Br or is absent when J₁ is N; G₆ may be selected from H, CH₃, OCH₃, CH₂CH₃, CF₃, F, Cl and Br or is absent when J₂ is N; G₇ may be selected from H, CH₃, OCH₃, CH₂CH₃, CF₃ and F or is absent when J₃ is N; G₈ may be selected from H, CH₃, OCH₃, CH₂CH₃ and F or is absent when J₄ is N; J₁ may be selected from C and N; J₂ may be selected from C and N; J₃ may be selected from C and N; J₄ may be selected from C and N; R₁ may be selected from

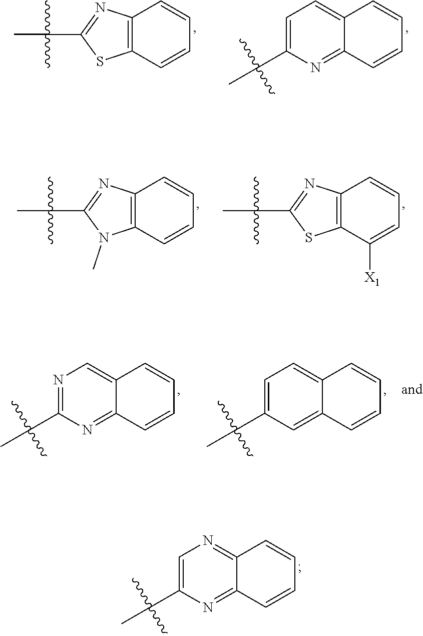

R₂ may be selected from

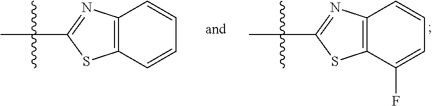 [SMILES: *c1nc2ccccc2s1 ; *c1nc2cccc(F)c2s1]

X₁ may be selected from F, Cl and Br; and provided that the compound is not

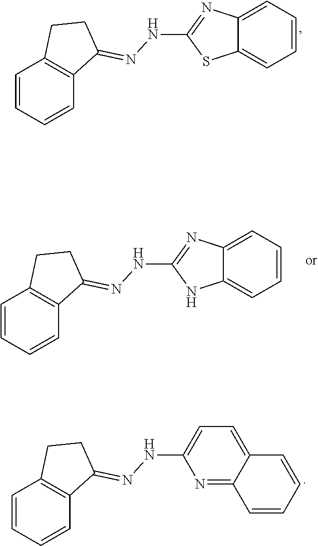 [SMILES: c1ccc2c(c1)CC/C2=N\Nc1nc2ccccc2s1 ; c1ccc2c(c1)CC/C2=N\Nc1nc2ccccc2[nH]1 ; c1ccc2c(c1)CC/C2=N\Nc1ccc2ccccc2n1]

G₁ may be selected from H, CH₃, CF₃, CN, OCH₃, F, Cl and Br; G₂ may be selected from H, OH, CH₃, CF₃,

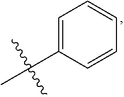 [SMILES: *c1ccccc1]

CN, OCH₃, OCHCH₃(CH₃), F, Cl and Br; G₃ may be selected from H, OH, CH₃, CF₃,

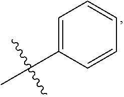 [SMILES: *c1ccccc1]

CN, OCH₃, OCHCH₃(CH₃), F, Cl and Br; and G₄ may be selected from H, CH₃, CF₃, CN, OCH₃, OCHCH₃(CH₃), F, Cl and Br.

G₁ may be selected from H, CF₃, F, Cl and Br; G₂ may be selected from H, CF₃,

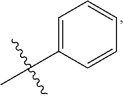 [SMILES: *c1ccccc1]

CN, OCHCH₃(CH₃), F, Cl and Br; G₃ may be selected from H, CF₃, CN, OCHCH₃(CH₃), F, Cl and Br; and G₄ may be selected from H, CF₃, CN, F, Cl, and Br.

R₁ may be selected from

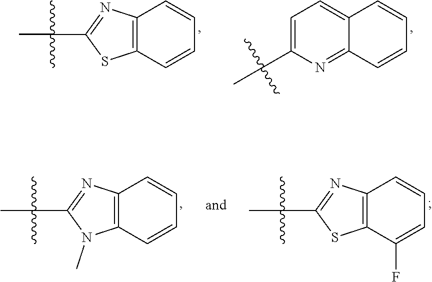 [SMILES: *c1nc2ccccc2s1 ; *c1ccc2ccccc2n1 ; *c1nc2ccccc2n1C ; *c1nc2cccc(F)c2s1]

and

R₂ may be selected from S and F

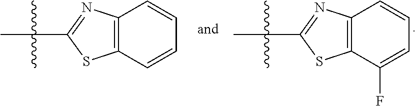 [SMILES: *c1nc2ccccc2s1 ; *c1nc2cccc(F)c2s1]

Z₁ may be C; Z₂ may be C; L₁ may be H; L₂ may be H; L₃ may be H; and L₄ may be H.

The compound may be selected from one or more of the following:

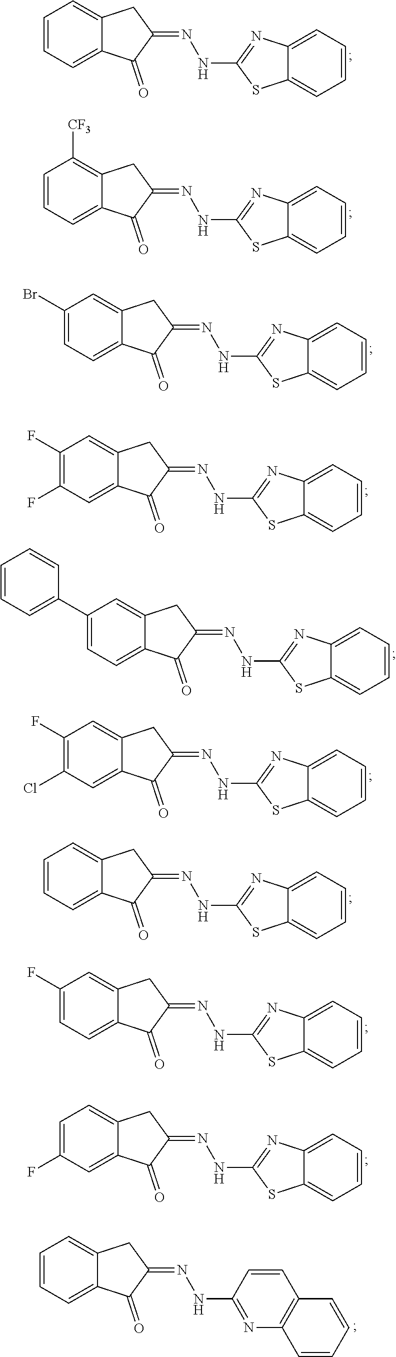

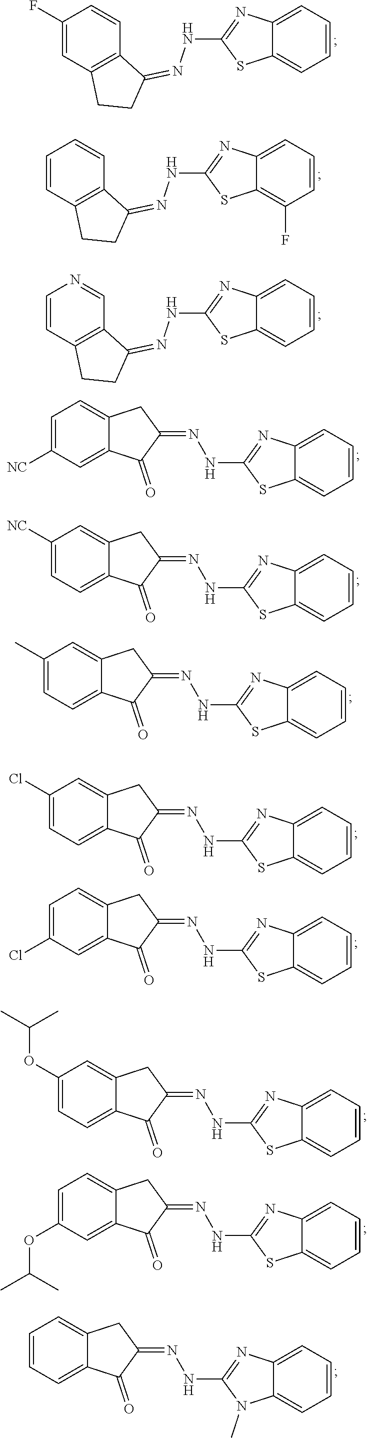

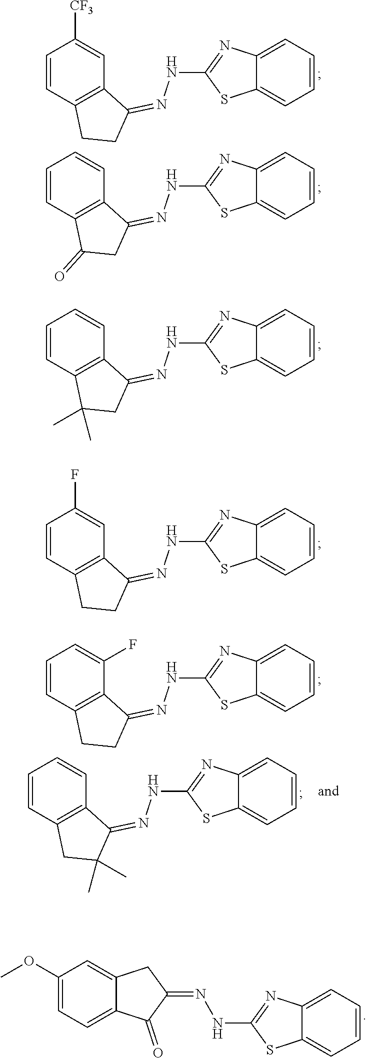

The compound may be selected from one or more of the following:

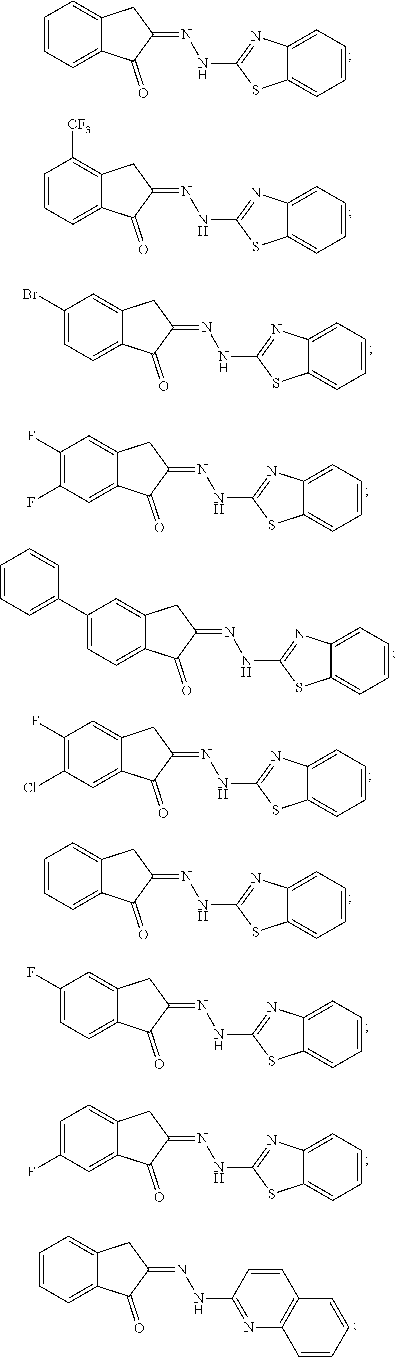

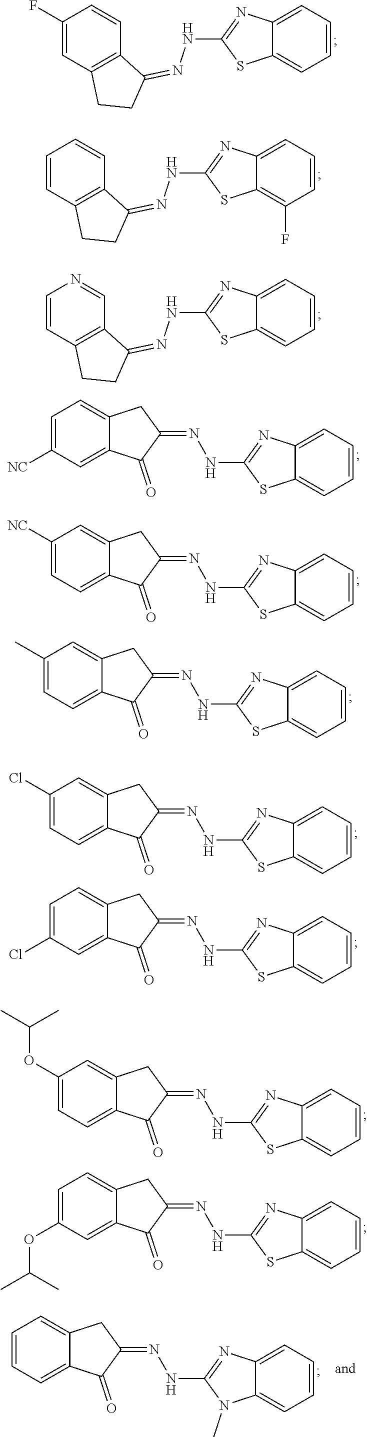

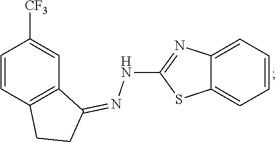 [SMILES: FC(F)(F)c1ccc2c(c1)/C(=N\Nc1nc3ccccc3s1)CC2]

The compound may be selected from one or more of the following:

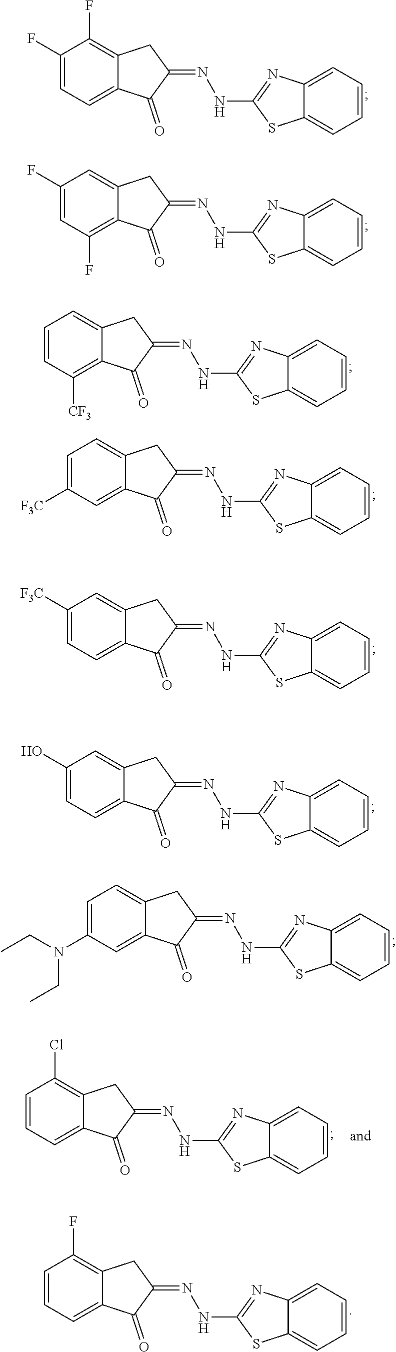

In accordance with a further aspect, there is provided a method of inhibiting POU domain transcription factor BRN2, the method comprising administering a compound as described herein and including the following compounds:

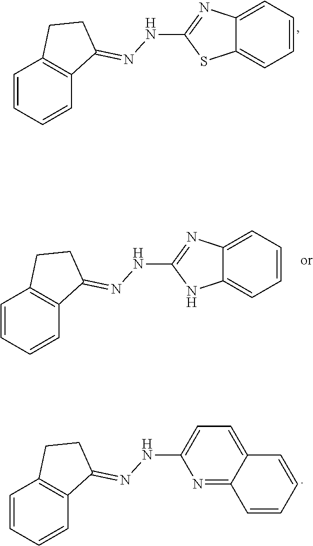 [SMILES: c1ccc2c(c1)CC/C2=N\Nc1nc2ccccc2s1 ; c1ccc2c(c1)CC/C2=N\Nc1nc2ccccc2[nH]1 ; c1ccc2c(c1)CC/C2=N\Nc1ccc2ccccc2n1]

In accordance with a further aspect, there is provided compound as described herein and including the following compounds: or

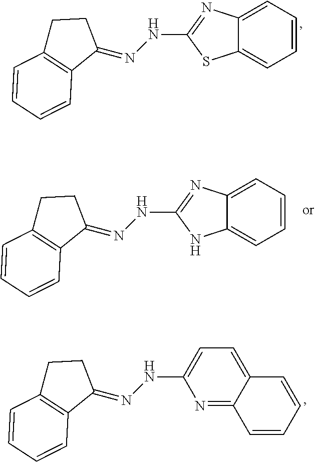 [SMILES: c1ccc2c(c1)CC/C2=N\Nc1nc2ccccc2s1 ; c1ccc2c(c1)CC/C2=N\Nc1nc2ccccc2[nH]1 ; c1ccc2c(c1)CC/C2=N\Nc1ccc2ccccc2n1]

for use in inhibiting POU domain transcription factor BRN2.

In accordance with a further aspect, there is provided pharmaceutical composition for treating cancer, comprising compound as described herein and a pharmaceutically acceptable carrier.

In accordance with a further aspect, there is provided a use of compound as described herein and including the following compounds:

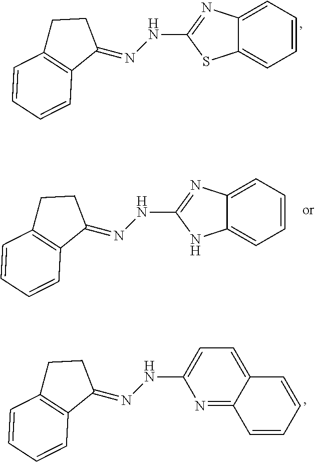 [SMILES: c1ccc2c(c1)CC/C2=N\Nc1nc2ccccc2s1 ; c1ccc2c(c1)CC/C2=N\Nc1nc2ccccc2[nH]1 ; c1ccc2c(c1)CC/C2=N\Nc1ccc2ccccc2n1]

for treating cancer.

In accordance with a further aspect, there is provided a use of compound as described herein and including the following compounds:

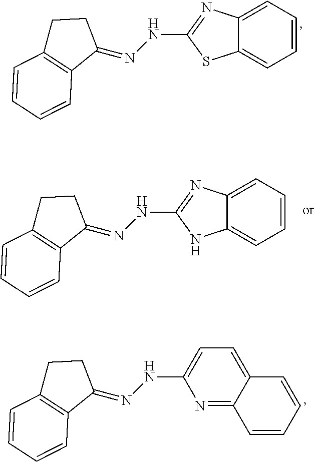 [SMILES: c1ccc2c(c1)CC/C2=N\Nc1nc2ccccc2s1 ; c1ccc2c(c1)CC/C2=N\Nc1nc2ccccc2[nH]1 ; c1ccc2c(c1)CC/C2=N\Nc1ccc2ccccc2n1]

in the manufacture of a medicament for treating cancer.

In accordance with a further aspect, there is provided a commercial package comprising (a) compound described herein and a pharmaceutically acceptable carrier; and (b) instructions for the use thereof for treating cancer.

In accordance with a further aspect, there is provided a commercial package comprising (a) a pharmaceutical composition comprising compound as described herein and including the following compounds:

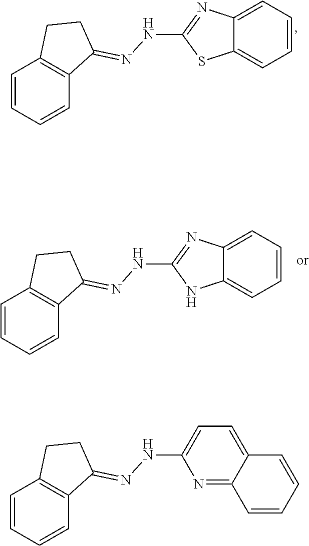 [SMILES: c1ccc2c(c1)CC/C2=N\Nc1nc2ccccc2s1 ; c1ccc2c(c1)CC/C2=N\Nc1nc2ccccc2[nH]1 ; c1ccc2c(c1)CC/C2=N\Nc1ccc2ccccc2n1]

and a pharmaceutically acceptable carrier; and (b) instructions for the use thereof for treating cancer.

G₁ may be selected from H, CH₃, CF₃, CN, OCH₃, NH₂, F, Cl and Br. G₂ may be selected from H, OH, CH₃, CF₃,

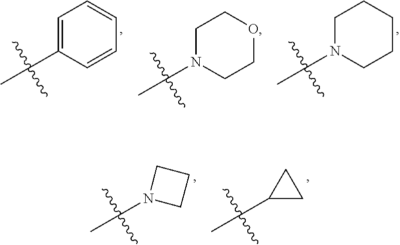

CN, OCH₃, OCHCH₃(CH₃), CH₂CH₃, CH(CH₃)CH₃, N(CH₃)CH₃, N(CH₂CH₃)CH₂CH₃, NH₂, tBuO₂C, F, Cl and Br. G₃ may be selected from H, OH, CH₃, CF₃,

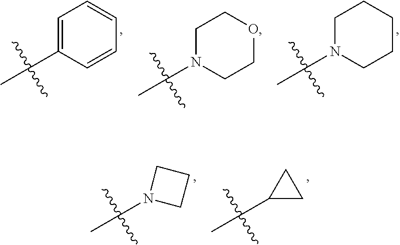

CN, OCH₃, OCHCH₃(CH₃), CH₂CH₃, CH(CH₃)CH₃, N(CH₃)CH₃, N(CH₂CH₃)CH₂CH₃, NH₂, tBuO₂C, F, Cl and Br. G₄ may be selected from H, CH₃, CF₃, CN, OCH₃, OCHCH₃(CH₃), CH₂CH₃, CH(CH₃)CH₃, N(CH₃)CH₃, N(CH₂CH₃)CH₂CH₃, NH₂, tBuO₂C, F, Cl and Br. G₁ may be selected from H, CF₃, F, Cl and Br. G₂ may be selected from H, CH₃,

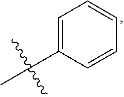 [SMILES: *c1ccccc1]

CN, OCH₃, OCHCH₃(CH₃), CH₂CH₃, F, Cl and Br. G₃ may be selected from H, CH₃, CF₃, CN, OCH₃, OCHCH₃(CH₃), CH₂CH₃, CH(CH₃)CH₃, F, Cl and Br. G₄ may be selected from H, CH₃, CF₃, CN, F, Cl and Br. G₅ may be selected from H, CH₃, CF₃, F, Cl and Br. G₆ may be selected from H, CH₃, CF₃, F, Cl and Br. G₇ may be selected from H, CH₃, CF₃ and F or may be absent when J₃ is N. G₈ may be selected from H, CH₃ and F. G₅ may be selected from H, F, Cl and Br. G₆ may be selected from H, F, Cl and Br. G₇ may be selected from H, CF₃ and F or may be absent when J₃ is N. G₈ may be selected from H, and F. R₁ may be selected from

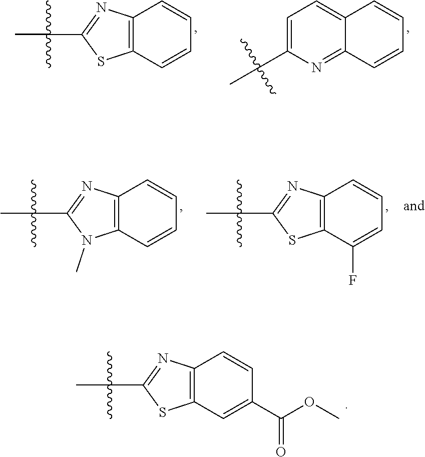 [SMILES: *c1nc2ccccc2s1 ; *c1ccc2ccccc2n1 ; *c1nc2ccccc2n1C ; *c1nc2cccc(F)c2s1 ; *c1nc2ccc(C(=O)OC)cc2s1]

R₂ may be selected from

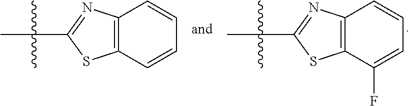 [SMILES: *c1nc2ccccc2s1 ; *c1nc2cccc(F)c2s1]

R₁ may be selected from

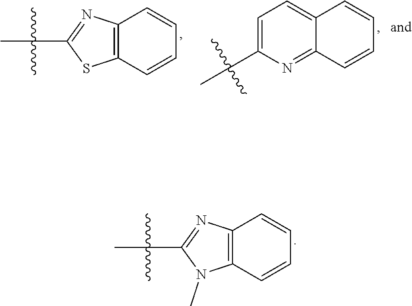 [SMILES: *c1nc2ccccc2s1 ; *c1ccc2ccccc2n1 ; *c1nc2ccccc2n1C]

R₂ may be selected from

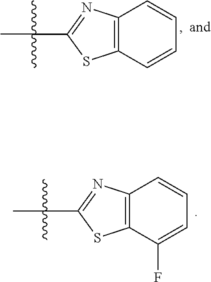 [SMILES: *c1nc2ccccc2s1 ; *c1nc2cccc(F)c2s1]

R₁ may be selected from

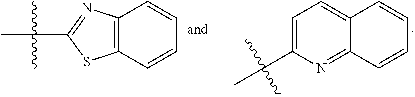 [SMILES: *c1nc2ccccc2s1 ; *c1ccc2ccccc2n1]

### R₁ may be

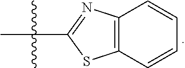 [SMILES: *c1nc2ccccc2s1]

may be

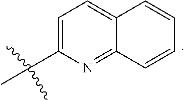 [SMILES: *c1ccc2ccccc2n1]

### R₁ may be

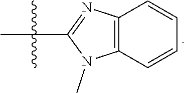 [SMILES: *c1nc2ccccc2n1C]

### R₂ may be

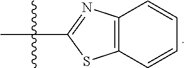 [SMILES: *c1nc2ccccc2s1]

### R₂ may be

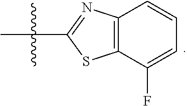 [SMILES: *c1nc2cccc(F)c2s1]

Z₁ may be selected from C and N. Z₂ may be selected from C and N. L₁ may be H or may be absent when Z₁ is N. L₂ may be H. L₃ may be H or may be absent when Z₂ is N. L₄ may be H. A₁ may be when O when [symbol] at a is a double bond or A₁ is two H when [symbol] at a is two single bonds; and A₂ may be two H when [symbol] at b is two single bonds. J₁ may be C. J₂ may be C. J₃ may be selected from C or N. J₄ may be C.

The cancer may be selected from one or more of the following: bladder cancer; cholangiocarcinoma; colorectal cancer; diffuse large B-cell lymphoma (DLBC); liver cancer; ovarian cancer; thymoma; thyroid cancer; clear cell renal cell carcinoma (CCRCC); chromophobe renal cell carcinoma (ChRCC); prostate cancer; breast cancer; uterine cancer; pancreatic cancer; cervical cancer; uveal melanoma; acute myeloid leukemia (AML); head and neck cancer; small cell lung cancer (SCLC); lung adenocarcinoma sarcoma; mesothelioma; adenoid cystic carcinoma (ACC); sarcoma; testicular germ cell cancer; uterine cancer; pheochromocytoma and paraganglioma (PCPG); melanoma; glioma; glioblastoma multiforme; T-cell Acute Lymphoblastic Leukemia; T-cell Lymphoma, medulloblastoma; and neuroblastoma.

The inhibiting of the POU domain transcription factor BRN2, may be for the treatment of cancer. The cancer may be a BRN2 expressing cancer. The cancer may be selected from the following cancers: prostate cancer; lung cancer; bladder cancer; sarcoma; glioma; and melanoma. The prostate cancer may be selected from: Neuroendocrine Prostate Cancer (NEPC); Prostate Adenocarcinoma; castration resistant prostate cancer (CRPC); androgen receptor pathway inhibitor (ARPI) resistant prostate cancer; enzalutamide (ENZ)-resistant (ENZ^{R}); and Abiraterone (Abi)-resistant (ABI_{R}). The lung cancer may be small cell lung cancer (SCLC) or lung adenocarcinoma. The bladder cancer may be small cell bladder cancer (SCBC). The sarcoma may be Ewing's sarcoma. The glioma may be glioblastoma multiforme.

The compound may be selected from one or more of the following:

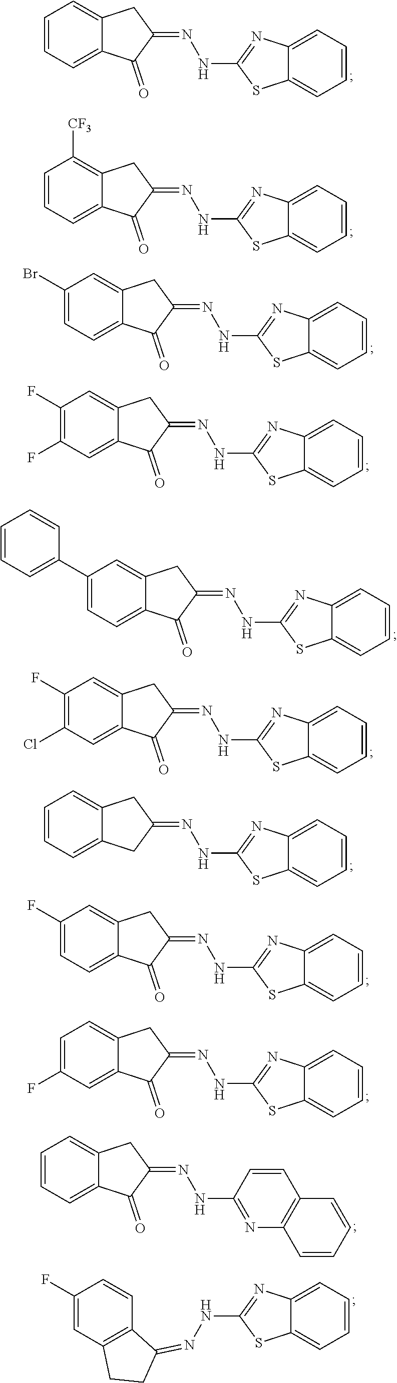

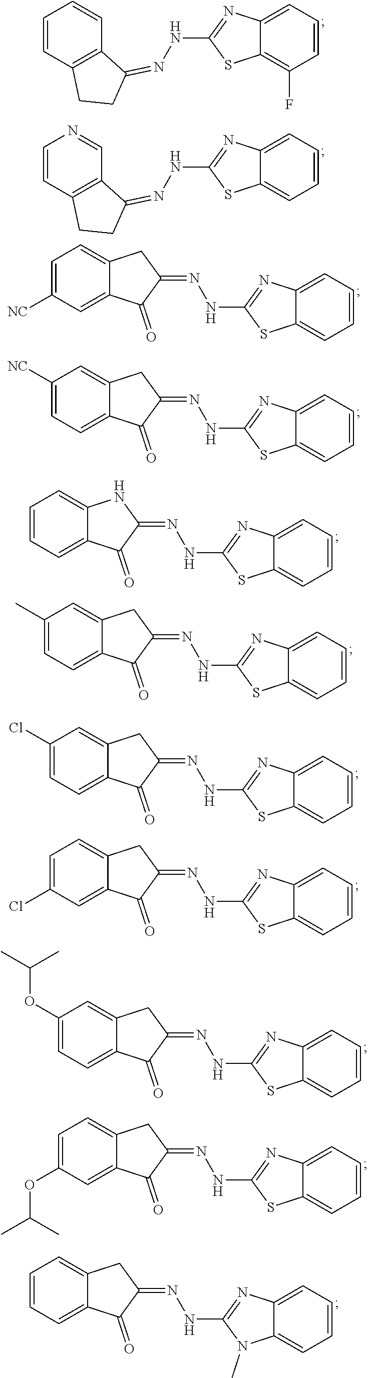

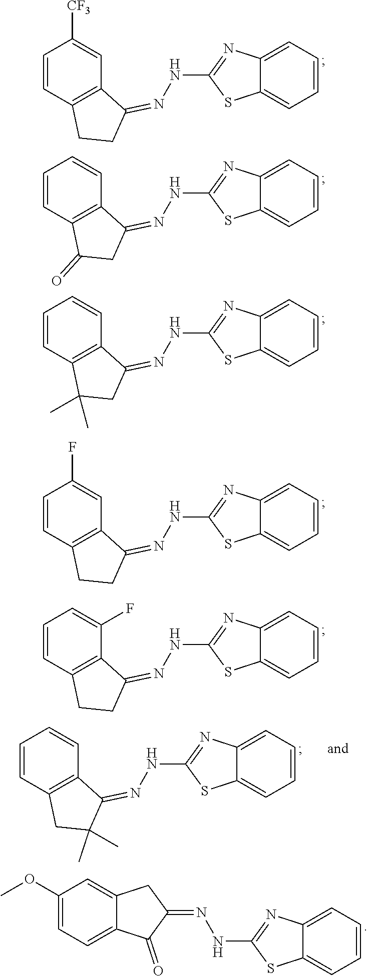

The compound may be selected from one or more of the following:

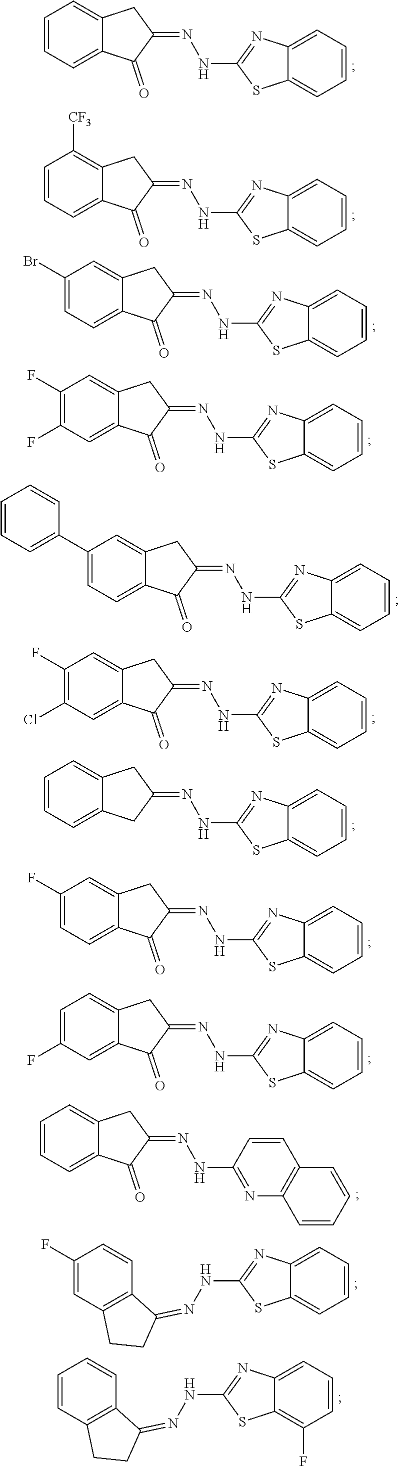

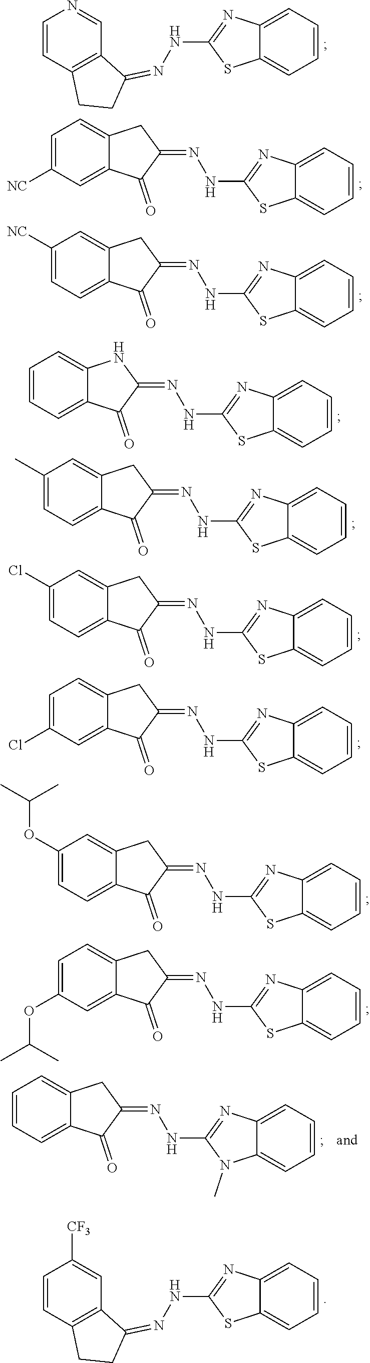

The compound may be selected from one or more of the following:

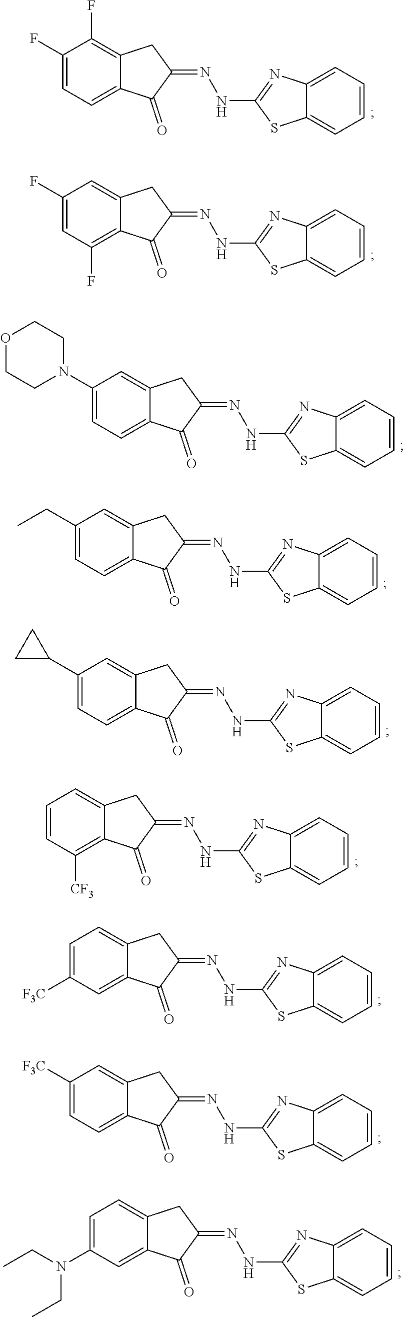

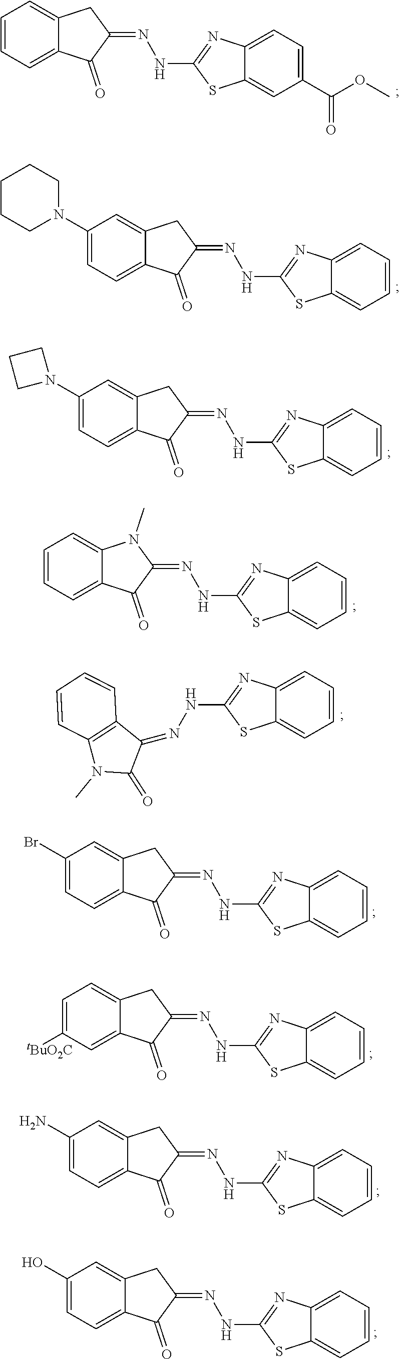

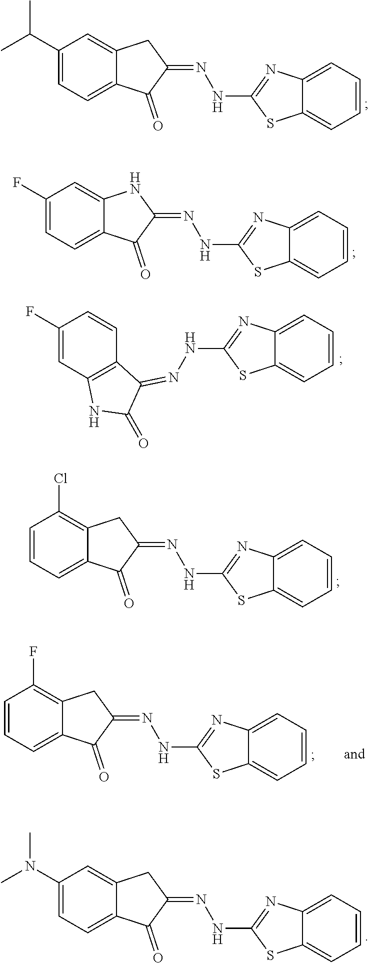

The compound may be selected from one or more of the following:

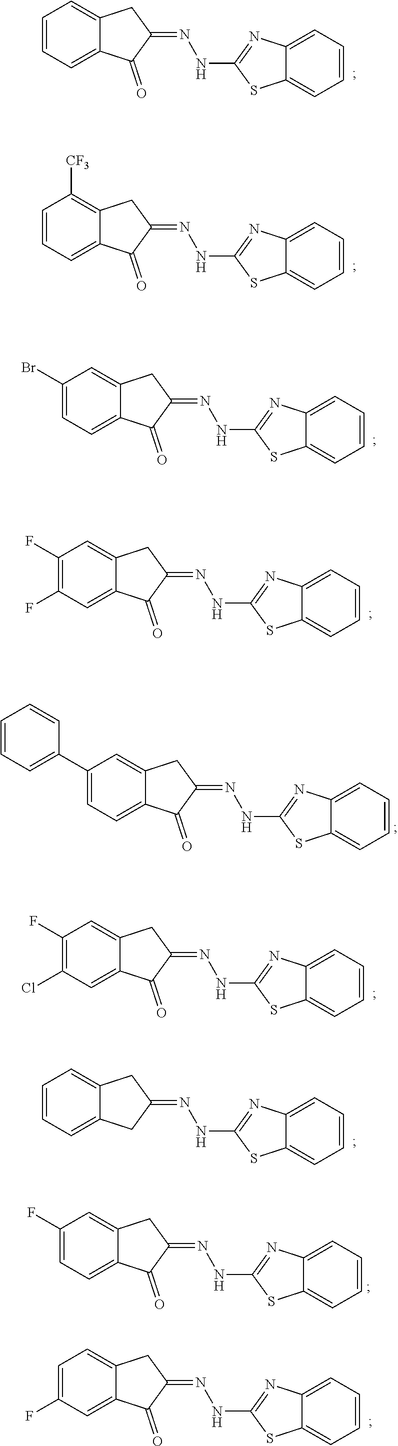

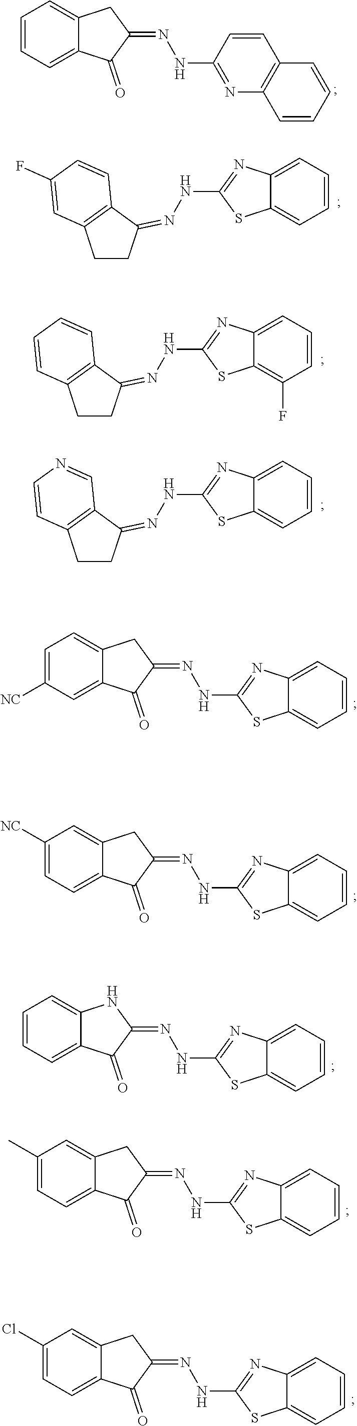

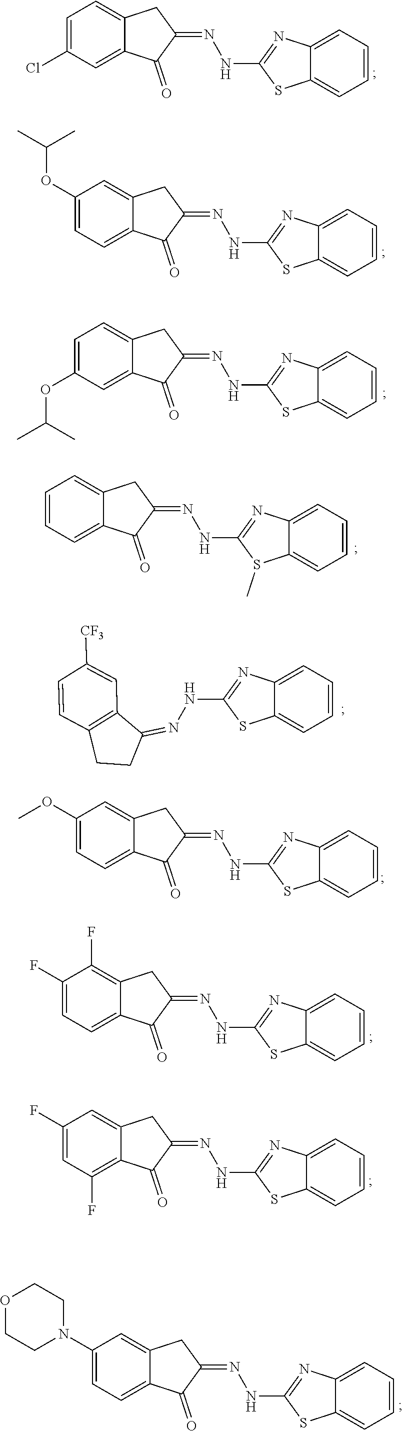

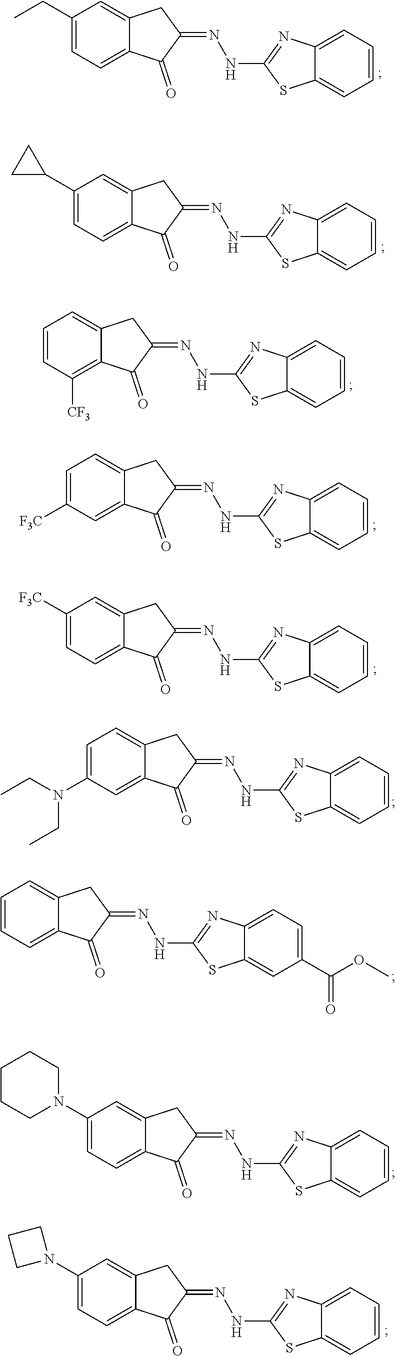

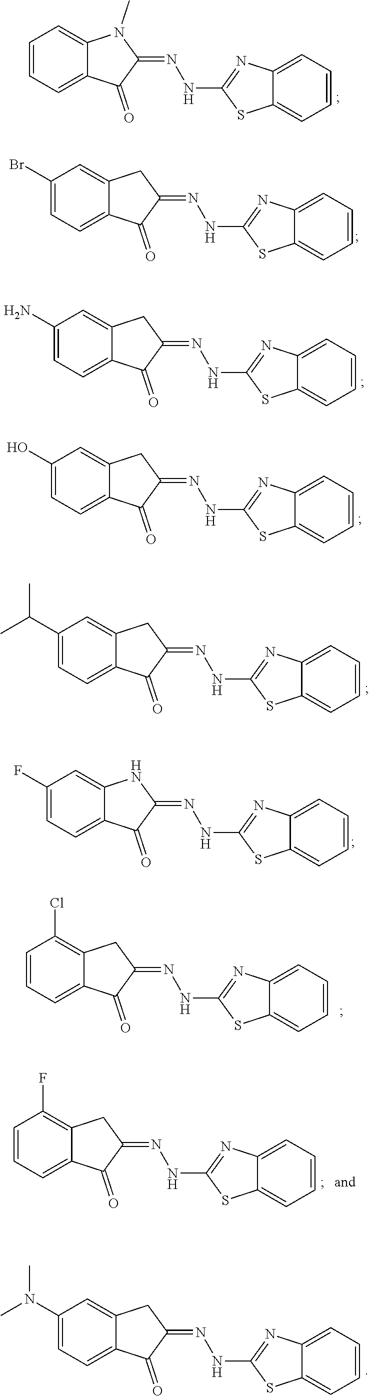

The compound may be selected from one or more of the compounds in TABLE 2.

In accordance with a further aspect, there is provided a method of inhibiting POU domain transcription factor BRN2, the method including administering a compound as described herein.

In accordance with a further aspect, there is provided a compound described herein, for use in inhibiting POU domain transcription factor BRN2.

In accordance with a further aspect, there is provided a pharmaceutical composition for treating cancer, including compound described herein and a pharmaceutically acceptable carrier.

In accordance with a further aspect, there is provided a use of compound described herein for treating cancer.

In accordance with a further aspect, there is provided a use of compound described herein for the manufacture of a medicament for treating cancer.

In accordance with a further aspect, there is provided a commercial package including (a) compound described herein and a pharmaceutically acceptable carrier; and (b) instructions for the use thereof for treating cancer.

In accordance with a further aspect, there is provided a commercial package including (a) a pharmaceutical composition described herein and a pharmaceutically acceptable carrier; and (b) instructions for the use thereof for treating cancer.

The inhibiting of the POU domain transcription factor BRN2, may be for the treatment of cancer. The cancer may be a BRN2 expressing cancer. The cancer may be selected from the following cancers: prostate cancer; lung cancer; bladder cancer; sarcoma; glioma; and melanoma. The prostate cancer may be selected from: Neuroendocrine Prostate Cancer (NEPC); Prostate Adenocarcinoma; castration resistant prostate cancer (CRPC); androgen receptor pathway inhibitor (ARPI) resistant prostate cancer; enzalutamide (ENZ)-resistant (ENZ^{R}); and Abiraterone (Abi)-resistant (ABI_{R}). The method of claim 17, wherein the lung cancer may be small cell lung cancer (SCLC) or lung adenocarcinoma. The bladder cancer may be small cell bladder cancer (SCBC). The sarcoma may be Ewing's sarcoma. The glioma may be glioblastoma multiforme. The BRN2 expressing cancer may be selected from the following: bladder cancer; cholangiocarcinoma; colorectal cancer; diffuse large B-cell lymphoma (DLBC); liver cancer; ovarian cancer; thymoma; thyroid cancer; clear cell renal cell carcinoma (CCRCC); chromophobe renal cell carcinoma (ChRCC); prostate cancer; breast cancer; uterine cancer; pancreatic cancer; cervical cancer; uveal melanoma; acute myeloid leukemia (AML); head and neck cancer; small cell lung cancer (SCLC); lung adenocarcinoma sarcoma; mesothelioma; adenoid cystic carcinoma (ACC); sarcoma; testicular germ cell cancer; uterine cancer; pheochromocytoma and paraganglioma (PCPG); melanoma; glioma; glioblastoma multiforme; T-cell Acute Lymphoblastic Leukemia; T-cell Lymphoma, medulloblastoma; and neuroblastoma.

### DETAILED DESCRIPTION

In silico computational drug discovery methods were used to conduct a virtual screen of ˜4 million purchasable lead-like compounds from the ZINC database (Irwin, J. et al. Abstracts of Papers Am. Chem. Soc. (2005) 230: U1009) to identify potential BRN2 binders. The in silico methods included large-scale docking, in-site rescoring and consensus voting procedures.

It will be understood by a person of skill that COOH and NR₂ may include the corresponding ions, for example carboxylate ions and ammonium ions, respectively. Alternatively, where the ions are shown, a person of skill in the art will appreciate that the counter ion may also be present.

Those skilled in the art will appreciate that the point of covalent attachment of the moiety to the compounds as described herein may be, for example, and without limitation, cleaved under specified conditions. Specified conditions may include, for example, and without limitation, in vivo enzymatic or non-enzymatic means. Cleavage of the moiety may occur, for example, and without limitation, spontaneously, or it may be catalyzed, induced by another agent, or a change in a physical parameter or environmental parameter, for example, an enzyme, light, acid, temperature or pH. The moiety may be, for example, and without limitation, a protecting group that acts to mask a functional group, a group that acts as a substrate for one or more active or passive transport mechanisms, or a group that acts to impart or enhance a property of the compound, for example, solubility, bioavailability or localization.

In some embodiments, compounds of Formula I or Formula II above may be used for systemic treatment of at least one indication selected from the group consisting of: prostate cancer, breast cancer, ovarian cancer, endometrial cancer, hair loss, acne, hirsutism, ovarian cysts, polycystic ovary disease, precocious puberty and age-related macular degeneration. In some embodiments compounds of Formula I or Formula II may be used in the preparation of a medicament or a composition for systemic treatment of an indication described herein. In some embodiments, methods of systemically treating any of the indications described herein are also provided.

Compounds as described herein may be in the free form or in the form of a salt thereof. In some embodiment, compounds as described herein may be in the form of a pharmaceutically acceptable salt, which are known in the art (Berge S. M. et al., J. Pharm. Sci. (1977) 66(1):1-19). Pharmaceutically acceptable salt as used herein includes, for example, salts that have the desired pharmacological activity of the parent compound (salts which retain the biological effectiveness and/or properties of the parent compound and which are not biologically and/or otherwise undesirable). Compounds as described herein having one or more functional groups capable of forming a salt may be, for example, formed as a pharmaceutically acceptable salt. Compounds containing one or more basic functional groups may be capable of forming a pharmaceutically acceptable salt with, for example, a pharmaceutically acceptable organic or inorganic acid. Pharmaceutically acceptable salts may be derived from, for example, and without limitation, acetic acid, adipic acid, alginic acid, aspartic acid, ascorbic acid, benzoic acid, benzenesulfonic acid, butyric acid, cinnamic acid, citric acid, camphoric acid, camphorsulfonic acid, cyclopentanepropionic acid, diethylacetic acid, digluconic acid, dodecylsulfonic acid, ethanesulfonic acid, formic acid, fumaric acid, glucoheptanoic acid, gluconic acid, glycerophosphoric acid, glycolic acid, hemisulfonic acid, heptanoic acid, hexanoic acid, hydrochloric acid, hydrobromic acid, hydriodic acid, 2-hydroxyethanesulfonic acid, isonicotinic acid, lactic acid, malic acid, maleic acid, malonic acid, mandelic acid, methanesulfonic acid, 2-napthalenesulfonic acid, naphthalenedisulphonic acid, p-toluenesulfonic acid, nicotinic acid, nitric acid, oxalic acid, pamoic acid, pectinic acid, 3-phenylpropionic acid, phosphoric acid, picric acid, pimelic acid, pivalic acid, propionic acid, pyruvic acid, salicylic acid, succinic acid, sulfuric acid, sulfamic acid, tartaric acid, thiocyanic acid or undecanoic acid. Compounds containing one or more acidic functional groups may be capable of forming pharmaceutically acceptable salts with a pharmaceutically acceptable base, for example, and without limitation, inorganic bases based on alkaline metals or alkaline earth metals or organic bases such as primary amine compounds, secondary amine compounds, tertiary amine compounds, quaternary amine compounds, substituted amines, naturally occurring substituted amines, cyclic amines or basic ion-exchange resins. Pharmaceutically acceptable salts may be derived from, for example, and without limitation, a hydroxide, carbonate, or bicarbonate of a pharmaceutically acceptable metal cation such as ammonium, sodium, potassium, lithium, calcium, magnesium, iron, zinc, copper, manganese or aluminum, ammonia, benzathine, meglumine, methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, glucamine, methylglucamine, theobromine, purines, piperazine, piperidine, procaine, N-ethylpiperidine, theobromine, tetramethylammonium compounds, tetraethylammonium compounds, pyridine, N,N-dimethylaniline, N-methylpiperidine, morpholine, N-methylmorpholine, N-ethylmorpholine, dicyclohexylamine, dibenzylamine, N,N-dibenzylphenethylamine, i-ephenamine, N,N′-dibenzylethylenediamine or polyamine resins.

In some embodiments, compounds as described herein may contain both acidic and basic groups and may be in the form of inner salts or zwitterions, for example, and without limitation, betaines. Salts as described herein may be prepared by conventional processes known to a person skilled in the art, for example, and without limitation, by reacting the free form with an organic acid or inorganic acid or base, or by anion exchange or cation exchange from other salts. Those skilled in the art will appreciate that preparation of salts may occur in situ during isolation and purification of the compounds or preparation of salts may occur by separately reacting an isolated and purified compound.

In some embodiments, compounds and all different forms thereof (e.g. free forms, salts, polymorphs, isomeric forms) as described herein may be in the solvent addition form, for example, solvates. Solvates contain either stoichiometric or non-stoichiometric amounts of a solvent in physical association the compound or salt thereof. The solvent may be, for example, and without limitation, a pharmaceutically acceptable solvent. For example, hydrates are formed when the solvent is water or alcoholates are formed when the solvent is an alcohol.

In some embodiments, compounds and all different forms thereof (e.g. free forms, salts, solvates, isomeric forms) as described herein may include crystalline and amorphous forms, for example, polymorphs, pseudopolymorphs, conformational polymorphs, amorphous forms, or a combination thereof. Polymorphs include different crystal packing arrangements of the same elemental composition of a compound. Polymorphs usually have different X-ray diffraction patterns, infrared spectra, melting points, density, hardness, crystal shape, optical and electrical properties, stability and/or solubility. Those skilled in the art will appreciate that various factors including recrystallization solvent, rate of crystallization and storage temperature may cause a single crystal form to dominate.

In some embodiments, compounds and all different forms thereof (e.g. free forms, salts, solvates, polymorphs) as described herein include isomers such as geometrical isomers, optical isomers based on asymmetric carbon, stereoisomers, tautomers, individual enantiomers, individual diastereomers, racemates, diastereomeric mixtures and combinations thereof, and are not limited by the description of the formula illustrated for the sake of convenience.

In some embodiments, pharmaceutical compositions as described herein may comprise a salt of such a compound, preferably a pharmaceutically or physiologically acceptable salt. Pharmaceutical preparations will typically comprise one or more carriers, excipients or diluents acceptable for the mode of administration of the preparation, be it by injection, inhalation, topical administration, lavage, or other modes suitable for the selected treatment. Suitable carriers, excipients or diluents (used interchangeably herein) are those known in the art for use in such modes of administration.

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water soluble compounds such as those used for vitamin K. For enteral administration, the compound may be administered in a tablet, capsule or dissolved in liquid form. The tablet or capsule may be enteric coated, or in a formulation for sustained release. Many suitable formulations are known, including, polymeric or protein microparticles encapsulating a compound to be released, ointments, pastes, gels, hydrogels, or solutions which can be used topically or locally to administer a compound. A sustained release patch or implant may be employed to provide release over a prolonged period of time. Many techniques known to one of skill in the art are described in Remington: the Science & Practice of Pharmacy by Alfonso Gennaro, 20^{th} ed., Lippencott Williams & Wilkins, (2000). Formulations for parenteral administration may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Compounds or pharmaceutical compositions as described herein or for use as described herein may be administered by means of a medical device or appliance such as an implant, graft, prosthesis, stent, etc. Also, implants may be devised which are intended to contain and release such compounds or compositions. An example would be an implant made of a polymeric material adapted to release the compound over a period of time.

An “effective amount” of a pharmaceutical composition as described herein includes a therapeutically effective amount or a prophylactically effective amount. A “therapeutically effective amount” refers to an amount effective, at dosages and for periods of time necessary, to achieve the desired therapeutic result, such as reduced tumor size, increased life span or increased life expectancy. A therapeutically effective amount of a compound may vary according to factors such as the disease state, age, sex, and weight of the subject, and the ability of the compound to elicit a desired response in the subject. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as smaller tumors, increased life span, increased life expectancy or prevention of the progression of prostate cancer to an androgen-independent form. Typically, a prophylactic dose is used in subjects prior to or at an earlier stage of disease, so that a prophylactically effective amount may be less than a therapeutically effective amount.

It is to be noted that dosage values may vary with the severity of the condition to be alleviated. For any particular subject, specific dosage regimens may be adjusted over time according to the individual need and the professional judgment of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In some embodiments, compounds and all different forms thereof as described herein may be used, for example, and without limitation, in combination with other treatment methods for at least one indication selected from the group consisting of: prostate cancer, breast cancer, ovarian cancer, endometrial cancer, hair loss, acne, hirsutism, ovarian cysts, polycystic ovary disease, precocious puberty and age-related macular degeneration. For example, compounds and all their different forms as described herein may be used as neo-adjuvant (prior), adjunctive (during), and/or adjuvant (after) therapy with surgery, radiation (brachytherapy or external beam), or other therapies (for example, HIFU).

In general, compounds as described herein should be used without causing substantial toxicity. Toxicity of the compounds as described herein can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be appropriate to administer substantial excesses of the compositions. Some compounds as described herein may be toxic at some concentrations. Titration studies may be used to determine toxic and non-toxic concentrations. Toxicity may be evaluated by examining a particular compound's or composition's specificity across cell lines using PC3 cells as a negative control that do not express AR. Animal studies may be used to provide an indication if the compound has any effects on other tissues. Systemic therapy that targets the AR will not likely cause major problems to other tissues since anti-androgens and androgen insensitivity syndrome are not fatal.

Compounds as described herein may be administered to a subject. As used herein, a “subject” may be a human, non-human primate, rat, mouse, cow, horse, pig, sheep, goat, dog, cat, etc. The subject may be suspected of having or at risk for having a cancer, such as prostate cancer, breast cancer, ovarian cancer or endometrial cancer, or suspected of having or at risk for having acne, hirsutism, alopecia, benign prostatic hyperplasia, ovarian cysts, polycystic ovary disease, precocious puberty, or age-related macular degeneration.

Diagnostic methods for various cancers, such as prostate cancer, breast cancer, ovarian cancer or endometrial cancer, and diagnostic methods for acne, hirsutism, alopecia, benign prostatic hyperplasia, ovarian cysts, polycystic ovary disease, precocious puberty, or age-related macular degeneration and the clinical delineation of cancer, such as prostate cancer, breast cancer, ovarian cancer or endometrial cancer, diagnoses and the clinical delineation of acne, hirsutism, alopecia, benign prostatic hyperplasia, ovarian cysts, polycystic ovary disease, precocious puberty, or age-related macular degeneration are known to those of ordinary skill in the art.

Definitions used include ligand-dependent activation of the androgen receptor (AR) by androgens such as dihydrotestosterone (DHT) or the synthetic androgen (R1881) used for research purposes. Ligand-independent activation of the AR refers to transactivation of the AR in the absence of androgen (ligand) by, for example, stimulation of the cAMP-dependent protein kinase (PKA) pathway with forskolin (FSK).

Some compounds and compositions as described herein may interfere with a mechanism specific to ligand-dependent activation (e.g., accessibility of the ligand binding domain (LBD) to androgen) or to ligand-independent activation of the AR.

Various alternative embodiments and examples of the invention are described herein. These embodiments and examples are illustrative and should not be construed as limiting the scope of the invention.

### Materials and Methods

### In Silico Screening

Modelling BRN2 protein structure: Since the crystal structure of human BRN2 has not been resolved, we built its structure using a homology modeling approach. Structurally, POU domain of BRN2 consists of two subdomains, a C-terminal homeodomain (POU_{H}) and N-terminal POU-specific region (POU_{S}) separated by a short non-conserved linker. Based on the sequence homology and crystal structure of other POU-domain proteins namely Piti, Oct1 and BRN5, we generated a BRN2 structure using Modeller9 (B. Webb, A. Sali. Comparative Protein Structure Modeling Using Modeller. Current Protocols in Bioinformatics 54, John Wiley & Sons, Inc., 5.6.1-5.6.37, 2016). The flexible loop that connects POU_{H} and POU_{S} domains was further refined by Loop Modeler module in MOE v2015. The quality of the developed BRN2 structure was assessed using Ramachandran plot and loons molecular dynamic (MD) simulations using AMBER (David Case, Thomas Cheatham, iii, Tom Darden, Holger Gohlke, Ray Luo, Kenneth Merz, jr., Alexey Onufriev, Carlos Simmerling, Bing Wang, Robert J. Woods. The Amber Biomolecular Simulation Programs. J Comput Chem. 2005 December; 26(16): 1668-1688.). The stability of BRN2 conformations was then assessed by the root mean squared deviation (RMSD) between the initial conformation and each snapshot during MD simulations.

In silico modeling for BRN2 Small molecule inhibitors: 4 millions of small-molecules from ZINC database v15 ((Teague Sterling, John J. Irwin. ZINC 15—Ligand Discovery for Everyone. J Chem Inf Model. 2015 55 (11): 2324-2337)) were docked into the active site of modeled hBRN2 structure using Glide (Friesner RA1, Banks J L, Murphy R B, Halgren T A, Klicic J J, Mainz D T, Repasky M P, Knoll E H, Shelley M, Perry J K, Shaw D E, Francis P, Shenkin P S. Glide: a new approach for rapid, accurate docking and scoring. 1. Method and assessment of docking accuracy. J Med Chem. 2004 Mar. 25; 47(7):1739-49.) and AutoDock (Stefano Forli, Ruth Huey, Michael E. Pique, Michel Sanner, David S. Goodsell, Arthur J. Olson. Computational protein-ligand docking and virtual drug screening with the AutoDock suite. Nat Protoc. 2016, 11(5): 905-919) programs. Next, RMSD values were calculated between the docking poses generated by Glide and AutoDock to identify the most consistent binding orientation of the compounds. Only molecules with poses having RMSD values below 2.0 Å were selected for further analysis. Furthermore, the selected ligands were subjected to additional on-site scoring using the LigX program and the pKi predicting module of the MOE. Finally, 2,000 compounds that consistently demonstrated high predicted binding affinities were selected for visual inspection to filter out compounds containing reactive or toxic groups such as aldehyde and alkyl halide groups. Based on chemical diversity and availability, 72 chemicals were purchased from commercial vendors as a first step of drugs testing.

### General Synthesis and Characterization of Compounds

### LCMS Conditions:

LCMS-Condition 01: Method: LCMS_X-Select (Formic acid)

Column: X-Select CSH C18 (3.0*50) mm 2.5μ; Mobile Phase: A: 0.05% Formic acid in water: ACN (95:5); B: 0.05% Formic acid in ACN; Injection volume: 2.0 μL; Column oven temperature: 50° C.; Flow Rate: 1.2.mL/minute; Gradient program: 0% B to 98% B in 2.0 minute, hold until 3.0 min, at 3.2 min B concentration is 0% up to 4.0 min.

### LCMS-Condition 02: Method: LCMS_X-Bridge (NH₃)

Column: X-Bridge C18 (3.0*50) mm 2.5μ; Mobile Phase: A: 0.05% NH₃ in water; B: 0.05% NH₃ in acetonitrile; Injection volume: 2.0 μL; Flow Rate: 1.0 mL/minute; Gradient program: 1% B to 90% B in 1.5 minute, 0% B in 2.5 minute, Hold until 2.8 minute, at 3.0 minute B concentration is 1% up to 4.0 min.

### LCMS-Condition 03: Method: LCMS_X-Select (Ammonium Bicarbonate)

Column: X-Select CSH C18 (3.0*50) mm 2.5p; Mobile Phase: A: 5 mM Ammonium Bicarbonate in water; B: Acetonitrile; Injection Volume: 2.0 μL, Flow Rate: 1.2 mL/minute; Column oven temp. 50° C.; Gradient program: 0% B to 98% B in 2.0 minute, hold until 3.0 min, at 3.2 min B concentration is 0% up to 4.0 min.

### LCMS-Condition 04: Method: LCMS_X-Select (TFA)

Column: X-Select CSH C18 (3.0*50) mm 2.5p; Mobile Phase: A: 0.1% TFA in water; B: Acetonitrile; Injection Volume: 2.0 μL, Flow Rate: 1.0 mL/minute; Gradient program: 2% B to 98% B in 2.8 minute, hold until 4.8 min, at 5.0 min B concentration is 2% up to 7.0 min.

### HPLC Conditions:

HPLC-Condition 01: Method: HPLC_X-Select (Formic acid):

Column: X-Select CSH C18 (4.6*150) mm 3.5p; Mobile Phase: A—0.1% Formic acid in water: Acetonitrile (95:05); B—Acetonitrile, Flow Rate: 1.0. mL/minute; Gradient program: Time (min)/B Conc.: 0.01/5, 1.0/5, 8.0/100, 12.0/100, 14.0/5, 18.0/5.

### HPLC-Condition 02: Method: HPLC_X-Select (Ammonium Bicarbonate):

Method:-HPLC_X-Bridge, Column: X-Bridge C18 (4.6*150) mm 5u, Mobile Phase: A—10 mMAmmonium bicarbonate in water, B—Acetonitrile, Inj Volume; 5.0 μL. Flow Rate: 1.0 mL/minute; Gradient program: Time(min)/B Conc.: 0.01/5, 1.0/5, 8.0/100, 12.0/100, 14.0/5, 18.0/5.

HPLC-Condition 03: Method: HPLC_X-Select (TFA):

Column: X-Select CSH C18 (4.6*150) mm 5 u, Mobile Phase: A—0.1% TFA in water, B—Acetonitrile Inj Volume; 5 μL, Flow Rate: 1.2. mL/minute; Gradient program: Time (min)/B Conc.: 0.01/5, 1.0/5, 8.0/100, 12.0/100, 14.0/5, 18.0/5.

### Synthesis of 7-62-2 (VPC 7-67)

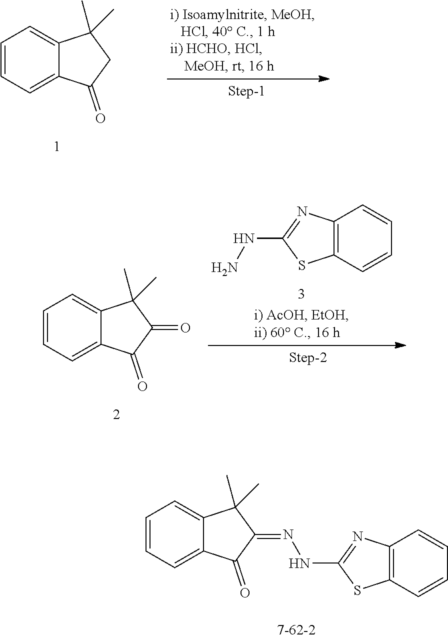 [SMILES: CC1(C)CC(=O)c2ccccc21 ; CC1(C)C(=O)C(=O)c2ccccc21 ; NNc1nc2ccccc2s1 ; CC1(C)/C(=N/Nc2nc3ccccc3s2)C(=O)c2ccccc21]

Step-1: Synthesis of 3,3-dimethyl-1H-indene-1,2(3H)-dione (2):

To a solution of 3,3-dimethyl-2,3-dihydro-1H-inden-1-one 1 (0.500 g, 3.125 mmol) in methanol (12 mL) was added isoamylnitrite (0.428 g, 3.437 mmol) followed by 37% HCl (3.8 mL) at room temperature and heated the reaction mixture at 40° C. for 1 h. After completion of the time, the resulting reaction mixture was concentrated under reduced pressure. The obtained residue was dissolved in 36% formaldehyde solution (2 mL) and stirred the reaction mixture at room temperature for further 16 h. After completion of the reaction (monitored by TLC and LCMS), the reaction mixture was cool to room temperature, quenched with water (50 mL) and extracted with ethyl acetate (2×25 mL). The combined organic layer was washed with brine (10 mL), dried over anhydrous Na₂SO₄, filtered and concentrated under reduced pressure and the crude compound was purified by Combiflash™ column chromatography eluting with 0-10% ethyl acetate in n-hexane to afford 0.300 g (55% yield) of 2 as an orange solid.

LCMS-Condition-10: [M+H]⁺=175.20; Rₜ=1.84 min

¹H NMR (400 MHz, DMSO-d₆) δ: 7.58-7.66 (m, 3H), 7.34-7.43 (m, 1H), 1.65 (br. s, 6H).

Step-2: Synthesis of (Z)-2-(2-(benzo[d]thiazol-2-yl)hydrazineylidene)-3,3-dimethyl-2,3-dihydro-1H-inden-1-one (7-62-2):

To a solution of 3,3-dimethyl-1H-indene-1,2(3H)-dione 2 (0.200 g, 1.149 mmol)) in ethanol (7.5 mL) was added 2-hydrazineylbenzo[d]thiazole 3 (0.208 g, 1.264 mmol) and heated the reaction mixture at 60° C. and stirred for 16 h. After completion of the reaction, the reaction mixture was cool to room temperature and was added 1-2 drops of acetic acid. The obtained solid was filtered through Buchner funnel and dried under reduced pressure. The crude obtained was purified by Prep-HPLC to afford 0.060 g, (16% yield) of 7-62-2 as an orange solid.

LCMS-Condition-1: [M+H]⁺=322.0; Rₜ=2.393 min.

¹H NMR (400 MHz, DMSO-d₆) δ: 13-45 (br. s, 1H), 7.77-7.94 (m, 2H), 7.65-7.75 (m, 2H), 7.39-7.54 (m, 2H), 7.14-7.32 (m, 2H), 1.55 (br. s, 6H).

HPLC-Condition-01: 95.94%, Rt: 10.50 min.

### Synthesis of 7-62-7 (VPC 7-68)

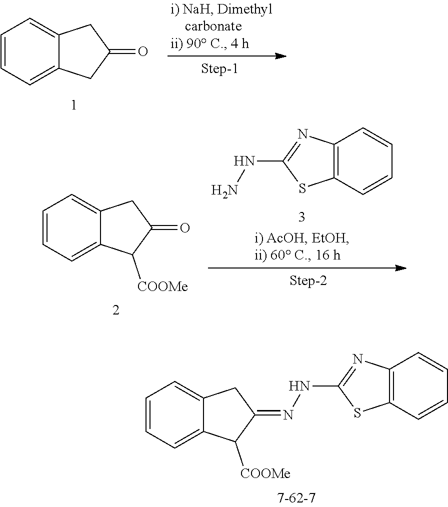 [SMILES: O=C1Cc2ccccc2C1 ; COC(=O)C1C(=O)Cc2ccccc21 ; NNc1nc2ccccc2s1 ; COC(=O)C1/C(=N/Nc2nc3ccccc3s2)Cc2ccccc21]

Step-1: Synthesis of methyl 2-oxo-2,3-dihydro-1H-indene-1-carboxylate (2):

To a solution of 1,3-dihydro-2H-inden-2-one 1(0.500 g, 3-48 mmol) in dimethyl carbonate (25 mL) was added NaH (0.136 g, 5.67 mmol) at room temperature and heated the reaction mixture at 90° C. for 4 h. After completion of the time, the resulting reaction mixture was concentrated under reduced pressure. After completion of the reaction (monitored by TLC and LCMS), the reaction mixture was quenched with ice water (20 mL) and extracted with ethyl acetate (3×20 mL). The combined organic layer was washed with brine (10 mL), dried over anhydrous Na₂SO₄, filtered and concentrated under reduced pressure and the crude compound was purified by Combiflash™ column chromatography eluting with 0-25% ethyl acetate in n-hexane to afford 0.140 g, (49% yield) of 2 as colourless viscous oil.

LCMS-Condition-01: [M+H]⁺=191.00; Rₜ=1.74 min

¹H NMR (400 MHz, DMSO-d₆) δ: 7.36-7.45 (m, 2H), 7.30-7.37 (m, 2H), 4.64 (br. s, 1H), 3.60 (br. s, 2H), 3.66 (s, 3H).

Step-2: Synthesis of methyl (E)-2-(2-(benzo[d]thiazol-2-yl)hydrazineylidene)-2,3-dihydro-1H-indene-1-carboxylate (7-62-7):

To a solution of methyl 2-oxo-2,3-dihydro-1H-indene-1-carboxylate 2 (0.200 g, 1.05 mmol) in ethanol (10 mL) was added 2-hydrazineylbenzo[d]thiazole 3 (0-191 g, 1.15 mmol) and heated the reaction mixture at 60° C. and stirred for 16 h. After completion of the reaction, the reaction mixture was cool to room temperature and was added 1-2 drops of acetic acid. The obtained solid was filtered through Buchner funnel and dried under reduced pressure. The crude obtained was purified by Prep-HPLC to afford 0.080 g, (23% yield) of 7-62-7 as Yellow solid.

LCMS-Condition-01: [M+H]⁺=337.9; Rₜ=2.036 min.

¹H NMR (400 MHz, DMSO-d₆) δ: 10.24 (br. s, 1H), 9.82 (s, 1H), 7.76 (d, J=7.99 Hz, 1H), 7.61 (d, J=7.99 Hz, 1H), 7.38-7.47 (m, 1H), 7.31 (t, J=15.9 Hz, 1H), 7.24 (d, J=7.99 Hz, 1H), 7.19 (t, J=15.9 Hz, 1H), 7.12 (t, J=15.9 Hz, 1H), 6.94 (t, J=15.9 Hz, 1H), 3.82 (s, 3H), 3.71 (s, 2H). HPLC-Condition-01: 96.02%, Rt: 9.083 min.

### Synthesis of 7-62-9 (VPC 7-69)

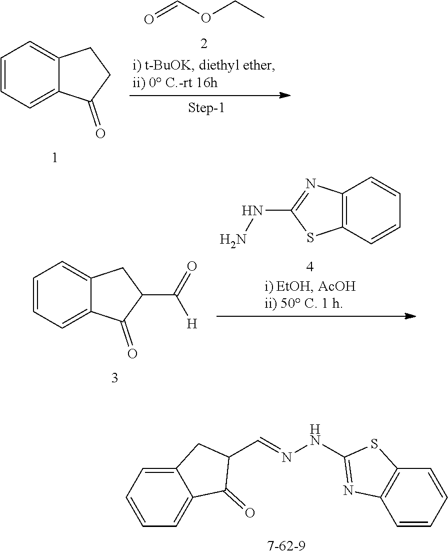

Step-1: Synthesis of 1-oxo-2,3-dihydro-1H-indene-2-carbaldehyde (3):

To a solution of 2,3-dihydro-1H-inden-1-one 1 (0.500 g, 3.783 mmol) and ethyl formate (0.560 g, 7.566 mmol) in Diethyl ether (15 mL) was added potassium tertiary butoxide (0.840 g, 7.566 mmol) at 0° C. and stirred for 30 min. The reaction mixture allowed to attain room temperature and stirred for further 16 h. After completion of the reaction (monitored by TLC and LCMS), the reaction mixture was concentrated under reduced pressure. The residue was dissolved diluted with water (5 mL) and neutralised with 1M HCl (1 mL). The obtained solid precipitate was filtered through Buchner funnel and dried under reduced pressure to afford 0.35 g (58% yield, crude) of 3 as brown solid. The crude compound was as such used for next step without carried out further purification. LCMS-Condition-01: [M+H]⁺=161.0; Rₜ=1.95 min

Step-2: Synthesis of (E)-2-((2-(benzo[d]thiazol-2-yl)hydrazineylidene)methyl)-2,3-dihydro-1H-inden-1-one (7-62-9):

To a solution of 1-oxo-2,3-dihydro-1H-indene-2-carbaldehyde 3 (0.100 g, 0.625 mmol) in ethanol (2 mL) was added 2-hydrazineylbenzo[d]thiazole 4 (0.100 g, 0.625 mmol) followed by acetic acid (2-3 drops) at room temperature and reaction mixture was heated at 50° C. and stirred for 1 h. After completion of the reaction (monitored by TLC and LCMS), the reaction mixture was cooled to room temperature, concentrated under reduced pressure and the crude compound was purified by Prep-HPLC to afford 0.04 g (21% yield) of 7-62-9 as yellow solid.

LCMS-Condition-01: [M+H]⁺=308.00; Rₜ=2.35 min.

¹H NMR (400 MHz, CD₃OD) δ: 8.10 (br. s, 1H), 7.61-7.76 (m, 3H), 7.43-7.51 (m, 2H), 7.27-7.39 (m, 2H), 7.10-7.24 (m, 1H), 3.62-3.73 (m, 2H), 3.56-3.68 (m, 1H), 3.35-3.49 (m, 1H).

HPLC-Condition-01: 95.04%, Rt: 8.422 min.

### Synthesis of 7-62-20 (VPC 7-62)

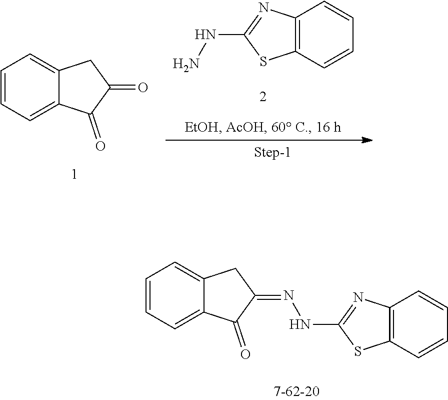 [SMILES: O=C1Cc2ccccc2C1=O ; NNc1nc2ccccc2s1 ; O=C1/C(=N\Nc2nc3ccccc3s2)Cc2ccccc21]

Step-1: Synthesis of (Z)-2-(2-(benzo[d]thiazol-2-yl)hydrazineylidene)-2,3-dihydro-1H-inden-1-one (7-62-20):

To a solution of 1H-indene-1,2(3H)-dione 1 (0.100 g, 0.684 mmol)) in ethanol (3 mL) was added 2-hydrazineylbenzo[d]thiazole 2 (0.110 g, 0.684 mmol) and heated the reaction mixture at 60° C. and stirred for 16 h. After completion of the reaction, the reaction mixture was cool to room temperature and was added 1-2 drops of acetic acid. The obtained solid was filtered through Buchner funnel and dried under reduced pressure. The crude obtained was purified by Prep-HPLC to afford (0.060 g, 16% yield) of 7-62-20 as yellow solid.

LCMS-Condition-01: [M+H]⁺=293.9; Rₜ=1.847 min. ¹H NMR (400 MHz, DMSO-d₆) δ: 12.48 (br. s, 1H), 7.20-7.85 (m, 8H), 3.88 (s, 3H). ¹H NMR (400 MHz, TFA) δ: 12.48 (br. s, 1H), 8.04-8.14 (m, 1H), 7.86-7.98 (m, 3H), 7.70-7.78 (m, 2H), 7.58-7.68 (m, 2H), 3.88 (s, 2H), 1.53 (s, 3H).

HPLC-Condition-01: 97.70%, Rt: 8.027 min.

### Synthesis of 7-62-29 (VPC 7-70)

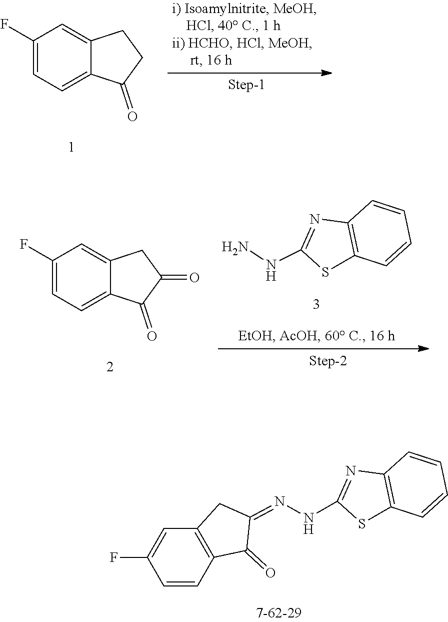 [SMILES: O=C1CCc2cc(F)ccc21 ; O=C1Cc2cc(F)ccc2C1=O ; NNc1nc2ccccc2s1 ; O=C1/C(=N\Nc2nc3ccccc3s2)Cc2cc(F)ccc21]

Step-1: Synthesis of 5-fluoro-1H-indene-1,2(3H)-dione (2):

To a solution of 5-fluoro-2,3-dihydro-1H-inden-1-one 1 (0.500 g, 3.330 mmol) in methanol (8 mL) was added isoamylnitrite (0.429 g, 3.663 mmol) followed by 37% HCl (1.5 mL) at room temperature and heated the reaction mixture at 40° C. for 1 h. After completion of the time, the resulting reaction mixture was concentrated under reduced pressure. The obtained residue was dissolved in 36% formaldehyde solution (2.1 mL) followed by 37% HCl (1.5 mL) and stirred the reaction mixture at room temperature for further 16 h. After completion of the reaction (monitored by TLC and LCMS), the reaction mixture was cool to room temperature, quenched with water (10 mL) and extracted with ethyl acetate (2×10 mL). The combined organic layer was washed with brine (10 mL), dried over anhydrous Na₂SO₄, filtered and concentrated under reduced pressure to afford 0.300 g (55% yield) of 2 as an off white solid. The crude was as such used for next reaction without carried out further purification.

LCMS-Condition-01: [M+H]⁺=165.10; Rₜ=1.94 min

Step-2: Synthesis of (Z)-2-(2-(benzo[d]thiazol-2-yl)hydrazineylidene)-5-fluoro-2,3-dihydro-1H-inden-1-one (7-62-29):

To a solution of 5-fluoro-1H-indene-1,2(3H)-dione 2 (0.200 g, 1.218 mol)) in ethanol (5 mL) was added 2-hydrazineylbenzo[d]thiazole 3 (0.201 g, 1.218 mmol) and heated the reaction mixture at 60° C. and stirred for 16 h. After completion of the reaction, the reaction mixture was cool to room temperature and was added 1-2 drops of acetic acid. The obtained solid was filtered through Buchner funnel and dried under reduced pressure. The crude was triturated with Ethanol (15 mL), dried under reduced pressure to afford 0.030 g, (8% yield) of 7-62-29 as Yellow solid.

LCMS-Condition-01: [M+H]⁺=311.9; Rₜ=1.816 min.

¹H NMR (400 MHz, DMSO-d₆) δ: 12.60 (br. s, 1H), 7.76-7.86 (m, 2H), 7.53 (d, J=7.89 Hz, 1H), 7.28-7.38 (m, 3H), 7.15-7.24 (m, 1H), 3.84 (s, 2H).

HPLC-Condition-02: 96.80%, Rt: 7.829 min.

### Synthesis of 7-62-30 (VPC 7-81)

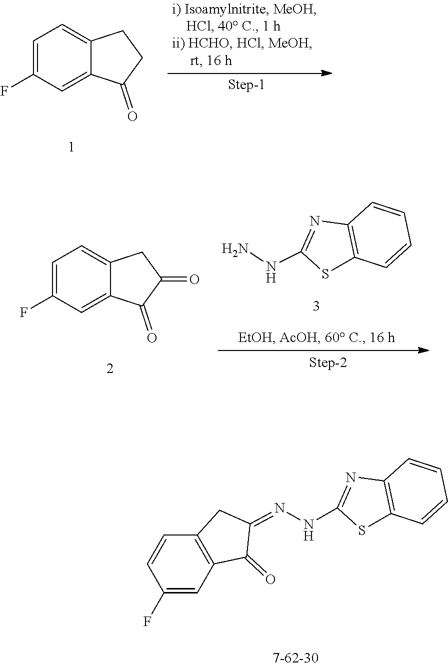 [SMILES: O=C1CCc2ccc(F)cc21 ; O=C1Cc2ccc(F)cc2C1=O ; NNc1nc2ccccc2s1 ; O=C1/C(=N\Nc2nc3ccccc3s2)Cc2ccc(F)cc21]

Step-1: Synthesis of 6-fluoro-1H-indene-1,2(3H)-dione (2):

To a solution of 6-fluoro-2,3-dihydro-1H-inden-1-one 1 (0.500 g, 3.330 mmol) in methanol (8 mL) was added isoamylnitrite (0.428 g, 3.437 mmol) followed by 37% HCl (1.5 mL) at room temperature and heated the reaction mixture at 40° C. for 1 h. After completion of the time, the resulting reaction mixture was concentrated under reduced pressure. The obtained residue was dissolved in 36% formaldehyde solution (2.1 mL) followed by 37% HCl (1.5 mL) at room temperature and stirred for further 16 h. After completion of the reaction (monitored by TLC and LCMS), the reaction mixture was cool to room temperature, quenched with water (50 mL) and extracted with ethyl acetate (2×25 mL). The combined organic layer was washed with brine (10 mL), dried over anhydrous Na₂SO₄, filtered and concentrated under reduced pressure and the crude compound was purified by Combiflash™ column chromatography eluting with 10-40% ethyl acetate in n-hexane to afford 0.250 g (46% yield) of 2 as an orange solid.

LCMS-Condition-01: [M+H]⁺=165.24; Rₜ=2.84 min

Step-2: Synthesis of (Z)-2-(2-(benzo[d]thiazol-2-yl)hydrazineylidene)-6-fluoro-2,3-dihydro-1H-inden-1-one (7-62-30):

To a solution of 6-fluoro-1H-indene-1,2(3H)-dione 2 (0.200 g, 1.218 mmol)) in ethanol (5 mL) was added 2-hydrazineylbenzo[d]thiazole 3 (0.201 g, 1.218 mmol) and heated the reaction mixture at 60° C. for 16 h. After completion of the reaction, the reaction mixture was cool to room temperature and was added (0.1 mL) of acetic acid. The obtained solid was filtered through Buchner funnel and dried under reduced pressure. The crude obtained was purified by Prep-HPLC to afford 0.050 g, (13% yield) of 7-62-30 as Yellow solid.

LCMS-Condition-01: [M+H]⁺=312.0; Rₜ=1.880 min.

¹H NMR (400 MHz, DMSO-d₆) δ: 12.61 (br. s, 1H), 7.83 (s, 1H), 7.65-7.75 (m, 1H), 7.50-7.60 (m, 3H), 7.28-7.38 (m, 1H), 7.15-7.25 (m, 1H), 3.84 (s, 2H).

HPLC-Condition-01: 95.25%, Rt: 8.545 min.

### Synthesis of 7-62-31 (VPC 7-71)

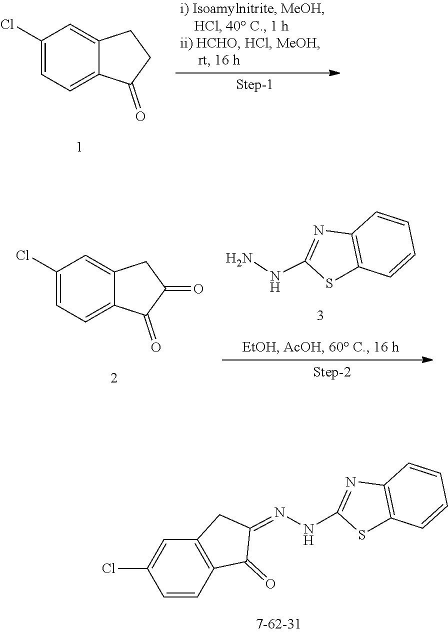 [SMILES: O=C1CCc2cc(Cl)ccc21 ; O=C1Cc2cc(Cl)ccc2C1=O ; NNc1nc2ccccc2s1 ; O=C1/C(=N\Nc2nc3ccccc3s2)Cc2cc(Cl)ccc21]

Step-1: Synthesis of 5-chloro-1H-indene-1,2(3H)-dione (2):

To a solution of 5-chloro-2,3-dihydro-1H-inden-1-one 1 (0.750 g, 4.501 mmol) in methanol (12 mL) was added isoamylnitrite (0.580 g, 4.951 mmol) followed by 37% HCl (0.5 mL) at room temperature and heated the reaction mixture at 40° C. for 1 h. After completion of the time, the resulting reaction mixture was concentrated under reduced pressure. The obtained residue was dissolved in 36% formaldehyde solution (1.6 mL) followed by 37% HCl (3.2 mL) and stirred the reaction mixture at room temperature for further 16 h. After completion of the reaction (monitored by TLC and LCMS), the reaction mixture was cool to room temperature, quenched with water (20 mL) and extracted with DCM (2×10 mL). The combined organic layer was washed with brine (10 mL), dried over anhydrous Na₂SO₄, filtered and concentrated under reduced pressure to afford 0.400 g (49% yield) of 2 as an off white solid. The crude was as such used for next reaction without carried out further purification.

LCMS-Condition-01: [M+H]⁺=190.10; Rₜ=2.034 min

Step-2: Synthesis (Z)-2-(2-(benzo[d]thiazol-2-yl)hydrazineylidene)-5-chloro-2,3-dihydro-1H-inden-1-one (7-62-31):

To a solution of 5-chloro-1H-indene-1,2(3H)-dione 2 (0.200 g, 1.107 mol)) in ethanol (5 mL) was added 2-hydrazineylbenzo[d]thiazole 3 (0.183 g, 1.107 mmol) and heated the reaction mixture at 60° C. and stirred for 16 h. After completion of the reaction, the reaction mixture was cool to room temperature and was added (0.1 mL) of acetic acid. The obtained solid was filtered through Buchner funnel and dried under reduced pressure. The crude was triturated with Ethanol (15 mL), dried under reduced pressure to afford 0.090 g, (25% yield) of 7-62-31 as Yellow solid.

LCMS-Condition-01: [M+H]⁺=327.71; Rₜ=1.939 min.

¹H NMR (400 MHz, DMSO-d₆) δ: 12.61 (br. s, 1H), 7.75-7.86 (m, 3H), 7.54 (d, J=7.89 Hz, 2H), 7.28-7.38 (m, 1H), 7.15-7.24 (m, 1H), 3.87 (s, 2H).

HPLC-Condition-02: 95.58%, Rt: 8.107 min.

### Synthesis of 7-62-32 (VPC 7-72)

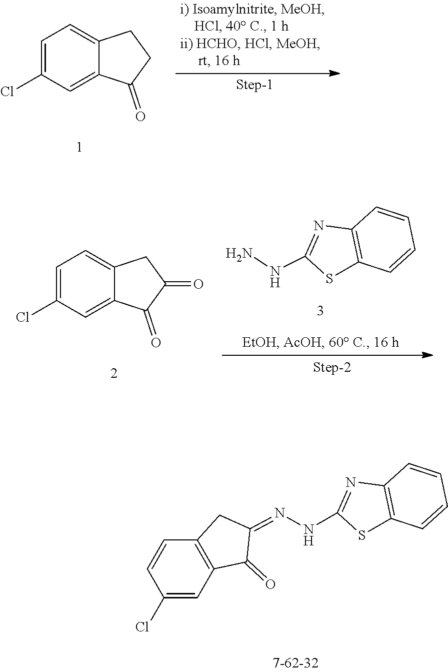 [SMILES: O=C1CCc2ccc(Cl)cc21 ; O=C1Cc2ccc(Cl)cc2C1=O ; NNc1nc2ccccc2s1 ; O=C1/C(=N\Nc2nc3ccccc3s2)Cc2ccc(Cl)cc21]

Step-1: Synthesis of 6-chloro-1H-indene-1,2(3H)-dione (2):

To a solution of 6-chloro-2,3-dihydro-1H-inden-1-one 1 (0.500 g, 3.001 mmol) in methanol (5 mL) was added isoamylnitrite (0.351 g, 3.001 mmol) followed by 37% HCl (1.5 mL) at room temperature and heated the reaction mixture at 40° C. for 1 h. After completion of the time, the resulting reaction mixture was concentrated under reduced pressure. The obtained residue was dissolved in 36% formaldehyde solution (2.2 mL) followed by 37% HCl (1.5 mL) and stirred the reaction mixture at room temperature for further 16 h. After completion of the reaction (monitored by TLC and LCMS), the reaction mixture was cool to room temperature, quenched with water (20 mL) and extracted with DCM (2×10 mL). The combined organic layer was washed with brine (10 mL), dried over anhydrous Na₂SO₄, filtered and concentrated under reduced pressure to afford 0.320 g (59% yield) of 2 as an off white solid. The crude was as such used for next reaction without carried out further purification.

LCMS-Condition-01: [M+H]⁺=181.20; Rₜ=2.048 min

Step-2: Synthesis 6-chloro-1H-indene-1,2(3H)-dione (7-62-32):

To a solution of 5-chloro-1H-indene-1,2(3H)-dione 2 (0.200 g, 1.107 mol)) in ethanol (5 mL) was added 2-hydrazineylbenzo[d]thiazole 3 (0.183 g, 1.107 mmol) and heated the reaction mixture at 60° C. and stirred for 16 h. After completion of the reaction, the reaction mixture was cool to room temperature and was added (0.1 mL) of acetic acid. The obtained solid was filtered through Buchner funnel and dried under reduced pressure. The crude was triturated with Ethanol (15 mL), dried under reduced pressure to afford 0.050 g, (14% yield) of 7-62-32 as Yellow solid.

LCMS-Condition-01: [M+H]⁺=327.95; Rₜ=3.120 min.

¹H NMR (400 MHz, DMSO-d₆) δ: 12.60 (br. s, 1H), 7.67-7.75 (m, 3H), 7.39-7.49 (m, 2H), 7.28-7.38 (m, 1H), 7.15-7.24 (m, 1H), 3.85 (s, 2H).

HPLC-Condition-03: 95.05%, Rt: 8.528 min.

### Synthesis of 7-62-39 (VPC 7-82)

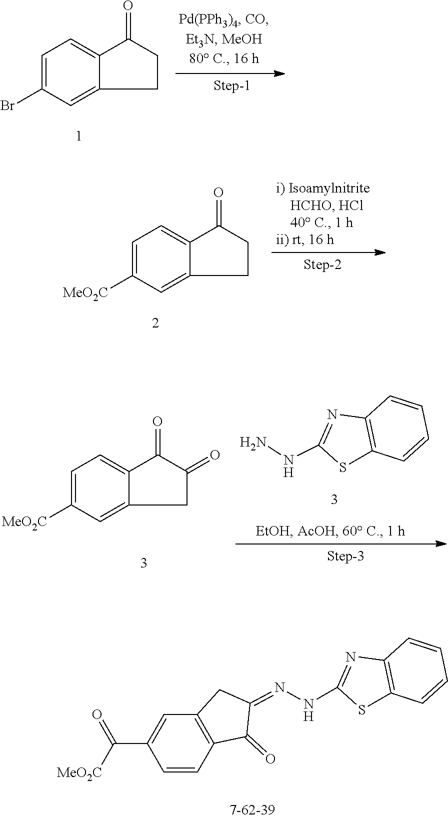 [SMILES: O=C1CCc2cc(Br)ccc21 ; COC(=O)c1ccc2c(c1)CCC2=O ; COC(=O)c1ccc2c(c1)CC(=O)C2=O ; NNc1nc2ccccc2s1 ; COC(=O)C(=O)c1ccc2c(c1)C/C(=N/Nc1nc3ccccc3s1)C2=O]

Step-1: Synthesis of methyl 1-oxo-2,3-dihydro-1H-indene-5-carboxylate (2):

To the stirred solution of 5-bromo-2,3-dihydro-1H-inden-1-one 1 (2.0 g, 9.474 mmol) in ethanol (30 mL) was added triethylamine (3.95 mL, 28.33 mmol) at room temperature and stirred for 15 min. To the resulting reaction mixture under inert atmosphere was added Pd(PPh₃)₄(1.1 g, 0.9474 mmol) at room temperature and the reaction mixture was stirred under CO gas atmosphere. The reaction mixture was sealed properly, heated at 80° C. temperature and stirred for 16 h. After completion of the reaction (monitored by TLC and LCMS), the reaction mixture was quenched with water (50 mL) and extracted with ethyl acetate (3×50 mL). The combined organic layer was washed with brine, dried over anhydrous Na₂SO₄, filtered and concentrated under reduced pressure and the crude compound was purified by Combiflash™ column chromatography eluting with 10-20% ethyl acetate in n-hexane to afford 1.8 g (99% yield) of 2 as off white solid.

LCMS-Condition-01: [M+H]⁺=191.20; Rₜ=1.92 min

Step-2: Synthesis of methyl 1,2-dioxo-2,3-dihydro-1H-indene-5-carboxylate (3):

To a solution of methyl 1-oxo-2,3-dihydro-1H-indene-5-carboxylate 2 (0.500 g, 1.051 mmol) in methanol (12 mL) was added isoamylnitrite (0.123 g, 1.051 mmol) followed by 37% HCl (0.5 mL) at room temperature and heated the reaction mixture at 40° C. for 1 h. After completion of the time, the resulting reaction mixture was concentrated under reduced pressure. The obtained residue was dissolved in 36% formaldehyde solution (1.6 mL) followed by 37% HCl (3.2 mL) and stirred the reaction mixture at room temperature for further 16 h. After completion of the reaction (monitored by TLC and LCMS), the reaction mixture was cool to room temperature, quenched with water (10 mL) and extracted with DCM (2×10 mL). The combined organic layer was washed with brine (10 mL), dried over anhydrous Na₂SO₄, filtered and concentrated under reduced pressure to afford 0.210 g (97% yield) of 3 as orange solid. The crude was as such used for next reaction without carried out further purification.

LCMS-Condition-01: [M+H]⁺=205.10; Rₜ=2.044 min

Step-3: Synthesis methyl (Z)-2-(2-(2-(benzo[d]thiazol-2-yl)hydrazineylidene)-1-oxo-2,3-dihydro-1H-inden-5-yl)-2-oxoacetate (7-62-39):

To a solution of methyl 1,2-dioxo-2,3-dihydro-1H-indene-5-carboxylate 2 (0.200 g, 0.979 mol) in ethanol (5 mL) was added 2-hydrazineylbenzo[d]thiazole 3 (0.161 g, 0.979 mmol) and heated the reaction mixture at 60° C. and stirred for 16 h. After completion of the reaction, the reaction mixture was cool to room temperature and was added (0.1 mL) of acetic acid. The obtained solid was filtered through Buchner funnel and dried under reduced pressure. The crude was triturated with Ethanol (15 mL), dried under reduced pressure to afford 0.050 g, (13% yield) of 7-62-39 as Yellow solid.

LCMS-Condition-01: [M+H]⁺=351.9; Rₜ=1.991 min.

¹H NMR (400 MHz, DMSO-d₆) δ: 12.62 (br. s, 1H), 8.19 (s, 1H), 8.03 (d, J=7.89 Hz, 1H) 7.38-7.49 (m, 2H)), 7.45-7.55 (m, 1H), 7.28-7.38 (m, 1H), 7.15-7.24 (m, 1H), 3.91 (s, 2H), 3.94 (s, 3H).

HPLC-Condition-03: 92.02%, Rt: 8.115 min.

### Synthesis of 7-62-43 (VPC 7-73)

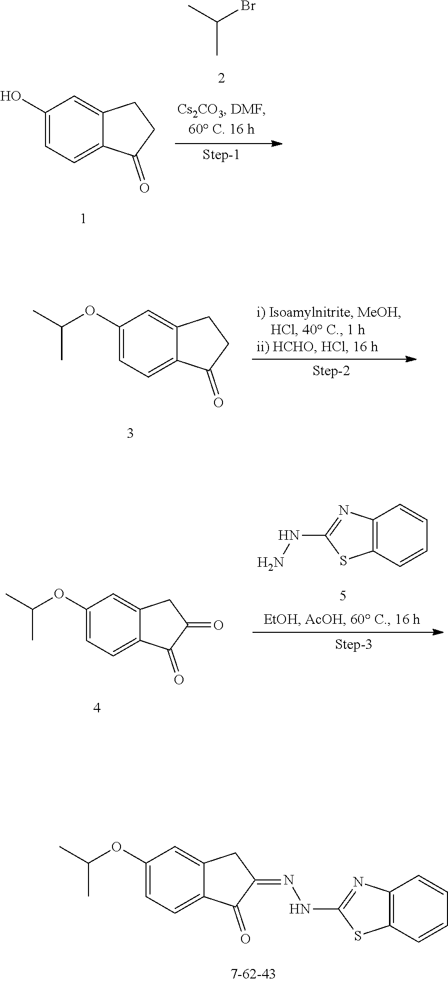

Step-1: Synthesis of 5-isopropoxy-2,3-dihydro-1H-inden-1-one (3):

To a solution of 5-hydroxy-2,3-dihydro-1H-inden-1-one 1 (0.500 g, 3.378 mmol) in DMF (3 mL) was added Cs₂CO₃ (2.2 g, 6.756 mmol) followed by 2-bromopropane 2 (0.830 g, 6.756 mmol) at room temperature and heated the reaction mixture at 60° C. for 16 h. After completion of the reaction (monitored by TLC and LCMS), the reaction mixture was quenched with ice cold water (20 mL) and extracted with diethyl ether (3×25 mL). The combined organic layer was washed with brine (10 mL), dried over anhydrous Na₂SO₄, filtered and concentrated under reduced pressure and the crude compound was purified by Combiflash™ column chromatography eluting with 10-20% ethyl acetate in n-hexane to afford 0.400 g (63% yield) of 3 as yellow solid.

LCMS-Condition-01: [M+H]⁺=191.00; Rₜ=1.84 min

Step-2: Synthesis of 5-isopropoxy-1H-indene-1,2(3H)-dione (4):

To a solution of 5-isopropoxy-2,3-dihydro-1H-inden-1-one 1 (0.400 g, 2.105 mmol) in methanol (6 mL) was added isoamylnitrite (0.270 g, 2.315 mmol) followed by 37% HCl (2 mL) at room temperature and heated the reaction mixture at 40° C. for 1 h. After completion of the time, the resulting reaction mixture was concentrated under reduced pressure. The obtained residue was dissolved in 36% formaldehyde solution (0.7 mL) followed by 37% HCl (2 mL) and stirred the reaction mixture at room temperature for further 16 h. After completion of the reaction (monitored by TLC and LCMS), the reaction mixture was cool to room temperature, quenched with water (10 mL) and extracted with DCM (3×10 mL). The combined organic layer was washed with brine (10 mL), dried over anhydrous Na₂SO₄, filtered and concentrated under reduced pressure and the crude compound was purified by Combiflash™ column chromatography eluting with 0-10% ethyl acetate in n-hexane to afford (0.350 g, Crude) of 4 as an orange solid. The crude was as such used for next reaction without carried out further purification.

LCMS-Condition-01: [M+H]⁺=205.00; Rₜ=1.920 min

Step-3: Synthesis of (Z)-2-(2-(benzo[d]thiazol-2-yl)hydrazineylidene)-5-isopropoxy-2,3-dihydro-1H-inden-1-one (7-62-43):

To a solution of 5-isopropoxy-1H-indene-1,2(3H)-dione 4 (0.100 g, 0.490 mmol)) in ethanol (3 mL) was added 2-hydrazineylbenzo[d]thiazole 4 (0.08 g, 0.490 mmol) and heated the reaction mixture at 60° C. and stirred for 16 h. After completion of the reaction, the reaction mixture was cool to room temperature and was added 1-2 drops of acetic acid and. The obtained solid was filtered through Buchner funnel and dried under reduced pressure. The crude obtained was purified by Prep-HPLC to afford 0.050 g (29% yield) of 7-62-43 as yellow solid

LCMS-Condition-01: [M+H]⁺=351.9; Rₜ=1.93 min.

¹H NMR (400 MHz, DMSO d₆) δ: 12.48 (br. s, 1H), 7.84 (br. s, 1H), 7.50-7.58 (m, 2H), 7.33-7.42 (m, 1H), 7.19-7.28 (m, 3H), 4.75 (q, J=23.99 Hz, 1H) 3.77 (s, 2H), 1.27 (d, J=7.99 Hz, 6H).

HPLC-Condition-01: 96.41%, Rt: 8.682 min.

### Synthesis of 7-62-44 (VPC 7-74)

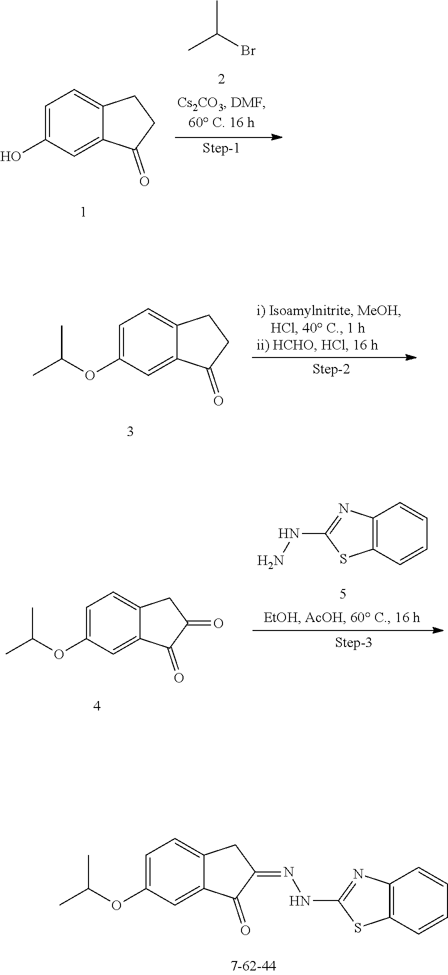

Step-1: Synthesis of 6-isopropoxy-2,3-dihydro-1H-inden-1-one (3):

To a solution of 6-hydroxy-2,3-dihydro-1H-inden-1-one 1 (0.500 g, 3.378 mmol) in DMF (3 mL) was added Cs₂CO₃ (2.2 g, 6.756 mmol) followed by 2-bromopropane 2 (0.830 g, 6.756 mmol) at room temperature and heated the reaction mixture at 60° C. for 16 h. After completion of the reaction (monitored by TLC and LCMS), the reaction mixture was quenched with ice cold water (10 mL) and extracted with ethyl acetate (2×20 mL). The combined organic layer was washed with brine (10 mL), dried over anhydrous Na₂SO₄, filtered and concentrated under reduced pressure and the crude compound was purified by Combiflash™ column chromatography eluting with 10-20% ethyl acetate in n-hexane to afford 0.400 g (62% yield) of 3 as yellow solid.

LCMS-Condition-01: [M+H]⁺=191.00; Rₜ=1.84 min.

Step-2: Synthesis of 6-isopropoxy-1H-indene-1,2(3H)-dione (4):

To a solution of 6-isopropoxy-2,3-dihydro-1H-inden-1-one 3 (0.400 g, 2.105 mmol) in methanol (6 mL) was added isoamylnitrite (0.270 g, 2.315 mmol) followed by 37% HCl (2 mL) at room temperature and heated the reaction mixture at 40° C. for 1 h. After completion of the time, the resulting reaction mixture was concentrated under reduced pressure. The obtained residue was dissolved in 36% formaldehyde solution (0.7 mL) followed by 37% HCl (2 mL) and stirred the reaction mixture at room temperature for further 16 h. After completion of the reaction (monitored by TLC and LCMS), the reaction mixture was cool to room temperature, quenched with water (10 mL) and extracted with DCM (3×10 mL). The combined organic layer was washed with brine (10 mL), dried over anhydrous Na₂SO₄, filtered and concentrated under reduced pressure and the crude compound was purified by Combiflash™ column chromatography eluting with 0-10% ethyl acetate in n-hexane to afford (0.300 g, Crude) of 4 as an off white solid. The crude was as such used for next reaction without carried out further purification.

LCMS-Condition-01: [M+H]⁺=205.30; Rₜ=1.948 min

Step-3: Synthesis of (Z)-2-(2-(benzo[d]thiazol-2-yl)hydrazineylidene)-6-isopropoxy-2,3-dihydro-1H-inden-1-one (7-62-44):

To a solution of 6-isopropoxy-1H-indene-1,2(3H)-dione 4 (0.100 g, 0.490 mmol) in ethanol (3 mL) was added 2-hydrazineylbenzo[d]thiazole 4 (0.08 g, 0.490 mmol) and heated the reaction mixture at 60° C. and stirred for 16 h. After completion of the reaction, the reaction mixture was cool to room temperature and was added 1-2 drops of acetic acid. The obtained solid was filtered through Buchner funnel and dried under reduced pressure. The crude compound obtained was purified by Prep-HPLC to afford 0.070 g (41% yield) of 7-62-44 as yellow solid.

LCMS-Condition-01: [M+H]⁺=351.9; Rₜ=1.93 min.

¹H NMR (400 MHz, DMSO d₆) δ: 12.48 (br. s, 1H), 7.84 (br. s, 1H), 7.50-7.58 (m, 2H), 7.33-7.42 (m, 1H), 7.19-7.28 (m, 3H), 4.75 (q, J=23.99 Hz, 1H) 3.77 (s, 2H), 1.27 (d, J=7.99 Hz, 6H).

HPLC-Condition-01: 98.32%, Rt: 9.292 min.

### Synthesis of 7-62-45 (VPC 7-7)

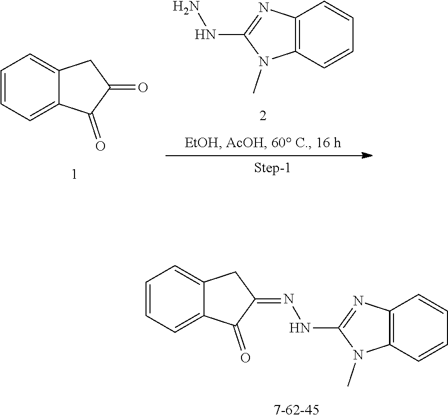 [SMILES: O=C1Cc2ccccc2C1=O ; Cn1c(NN)nc2ccccc21 ; Cn1c(N/N=C2/Cc3ccccc3C2=O)nc2ccccc21]

Step-1: Synthesis of (Z)-2-(2-(1-methyl-1H-benzo[d]imidazol-2-yl)hydrazineylidene)-2,3-dihydro-1H-inden-1-one (7-62-45):

To the stirred solution of 1H-indene-1,2(3H)-dione 1 (0.300 g, 2.052 mmol) in ethanol (15 mL) was added 2-hydrazineyl-1-methyl-1H-benzo[d]imidazole 2 (0.332 g, 2.052 mmol) followed by acetic acid (0.1 mL) at room temperature and reaction mixture was stirred at 60° C. temperature for 16 h. After completion of the reaction (monitored by TLC and LCMS), the reaction mixture was cooled to room temperature and solid precipitate was filtered through Buchner funnel. Dissolved the obtained solid in ethanol (15 mL) and concentrated under reduced pressure to afford 0.070 g (12% yield) of 7-62-45 as light yellow solid.

LCMS-Condition-01: [M+H]⁺=291.1; Rt=1.808 min.

¹H NMR (400 MHz, DMSO-d₆) δ: 11-33 (br. s, 1H), 7.63-7.75 (m, 3H), 7.38-7.48 (m, 2H), 7.29 (d, J=7.89 Hz, 1H) 7.10-7.20 (m, 2H), 3.88 (s, 2H), 3.64 (s, 2H).

HPLC-Condition-03: 96.90%, Rt: 6.152 min.

### Synthesis of 7-62-46 (VPC 7-76)

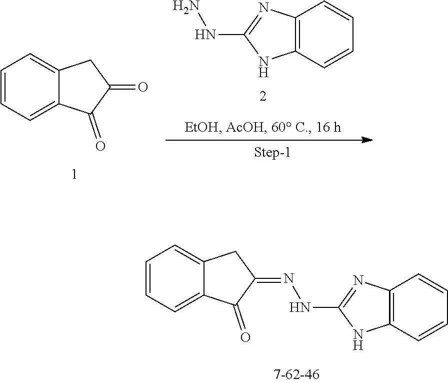 [SMILES: O=C1Cc2ccccc2C1=O ; NNc1nc2ccccc2[nH]1 ; O=C1/C(=N\Nc2nc3ccccc3[nH]2)Cc2ccccc21]

Step-1: Synthesis of (Z)-2-(2-(1H-benzo[d]imidazol-2-yl)hydrazineylidene)-2,3-dihydro-1H-inden-1-one (7-62-46):

To the stirred solution of 1H-indene-1,2(3H)-dione 1 (0.300 g, 2.052 mmol) in ethanol (15 mL) was added 2-hydrazineyl-1H-benzo[d]imidazole 2 (0.304 g, 2.052 mmol) followed by acetic acid (0.1 mL) at room temperature and reaction mixture was stirred at 60° C. temperature for 16 h. After completion of the reaction (monitored by TLC and LCMS), the reaction mixture was cooled to room temperature and solid precipitate was filtered through Buchner funnel.

Dissolved the obtained solid in ethanol (15 mL) and concentrated under reduced pressure to afford 0.090 g (16% yield) of 7-62-46 as yellow solid.

LCMS-Condition-03: [M+H]⁺=277.0; Rt=1.613 min.

¹H NMR (400 MHz, DMSO-d₆) δ: 11.88 (br. s, 2H), 7.66-7.76 (m, 2H), 7.63 (d, J=7.99 Hz, 1H), 7.40-7.50 (m, 1H), 7.20-7.30 (m, 2H), 7.02-7.08 (m, 2H), 3.93 (s, 2H).

HPLC-Condition-03: 97.01%, Rt: 6.063 min.

### Synthesis of 7-62-47 (VPC 7-77)

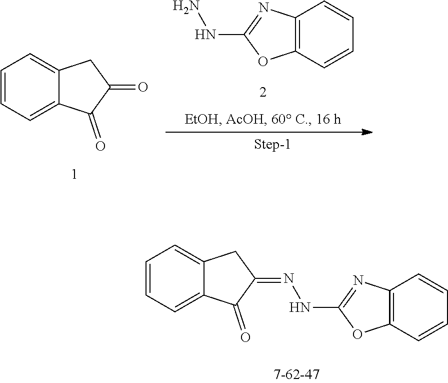 [SMILES: O=C1Cc2ccccc2C1=O ; NNc1nc2ccccc2o1 ; O=C1/C(=N\Nc2nc3ccccc3o2)Cc2ccccc21]

Step-1: Synthesis of (Z)-2-(2-(benzo[d]oxazol-2-yl)hydrazineylidene)-2,3-dihydro-1H-inden-1-one (7-62-47):

To the stirred solution of 1H-indene-1,2(3H)-dione 1 (0.300 g, 2.052 mmol) in ethanol (15 mL) was added 2-hydrazineylbenzo[d]oxazole 2 (0.306 g, 2.052 mmol) followed by acetic acid (0.1 mL) at room temperature and reaction mixture was stirred at 60° C. temperature for 16 h. After completion of the reaction (monitored by TLC and LCMS), the reaction mixture was cooled to room temperature and solid precipitate was filtered through Buchner funnel. Dissolved the obtained solid in ethanol (15 mL) and concentrated under reduced pressure to afford 0.065 g (11% yield) of 7-62-47 as yellow solid.

LCMS-Condition-04: [M+H]⁺=278.05; Rt=2.782 min.

¹H NMR (400 MHz, DMSO-d₆) δ: 12.22 (br. s, 1H), 7.62-7.76 (m, 3H), 7.46-7.56 (m, 2H), 7.35-7.45 (m, 1H), 7.15-7.27 (m, 2H), 3.93 (s, 2H).

HPLC-Condition-01: 97.40%, Rt: 7.578 min.

### Synthesis of 7-62-50 (VPC 7-83)

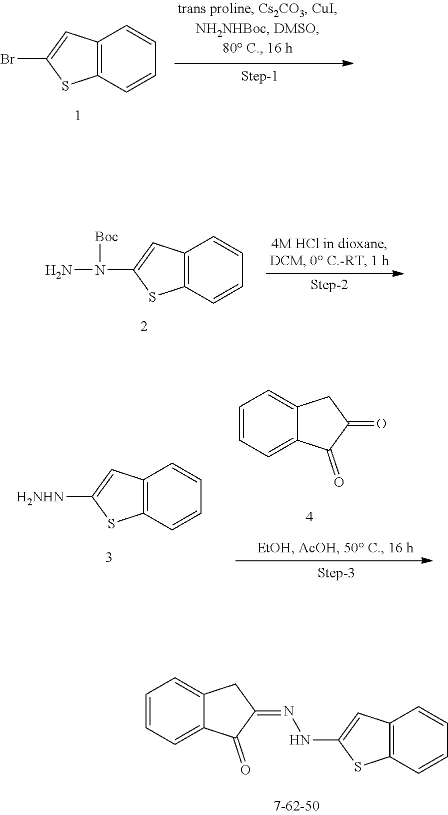

Step-2: Synthesis of benzo[b]thiophen-2-ylhydrazine (3):

To a solution of tert-butyl 1-(benzo[b]thiophen-2-yl) hydrazine-1-carboxylate 2 (0.100 g, 0.370 mmol) in DCM (1 mL) was added 4M HCl in dioxane (1 mL) 0° C. temperature. The reaction mixture was allowed to attain room temperature and stirred for 1 h. After completion of the reaction (monitored by TLC and LCMS), the reaction mixture was concentrated under reduced pressure and obtained residue was dissolved in water (5 mL), basified with saturated NaHCO₃ solution (2 mL) and extracted with ethyl acetate (2×10 mL). The combined organic layer was washed with brine (10 mL), dried over anhydrous Na₂SO₄, filtered and concentrated under reduced pressure to afford 0.04 g (64% yield) of 3 as brown solid. The crude was as such used for next reaction without carried out further purification. ¹H NMR (400 MHz, DMSO-d₆) δ: 8.30 (br. s, 1H), 7.80-7.90 (m, 2H), 7.23-7.32 (m, 2H), 7.38-7.50 (m, 1H), 4.60 (s, 2H).

Step-3: Synthesis (Z)-2-(2-(benzo[b]thiophen-2-yl)hydrazineylidene)-2,3-dihydro-1H-inden-1-one (7-62-50):

To a solution of benzo[b]thiophen-2-ylhydrazine 3 (0.040 g, 0.243 mol) in ethanol (2 mL) was added 1H-indene-1,2(3H)-dione 4 (0.035 g, 0.243 mmol) and heated the reaction mixture at 60° C. and stirred for 16 h. After completion of the reaction, the reaction mixture was cool to room temperature and was added 1-2 drops of acetic acid. The obtained solid was filtered through Buchner funnel and dried under reduced pressure. The crude was triturated with Ethanol (1 mL), dried under reduced pressure to afford 0.020 g, (28% yield) of 7-62-50 as Yellow solid.

LCMS-Condition-03: [M+H]⁺=292.9; Rₜ=2.229 min. ¹H NMR (400 MHz, DMSO-d₆) δ: 12.61 (br. s, 1H), 8.26 (d, J=7.89 Hz, 1H), 8.98-8.11 (m, 4H), 7.75-7.85 (m, 2H), 7.60-7.70 (m, 2H), 4.19 (s, 2H).

HPLC-Condition-03: 98.84%, Rt: 9.503 min.

### Synthesis of 7-62-51 (VPC 7-80)

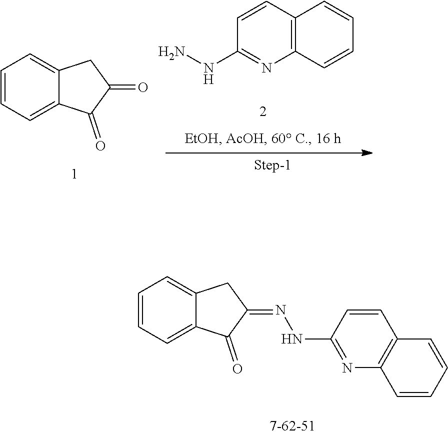 [SMILES: O=C1Cc2ccccc2C1=O ; NNc1ccc2ccccc2n1 ; O=C1/C(=N\Nc2ccc3ccccc3n2)Cc2ccccc21]

Step-1: Synthesis of (Z)-2-(2-(quinolin-2-yl) hydrazineylidene)-2,3-dihydro-1H-inden-1-one (7-62-51):

To the stirred solution of 1H-indene-1,2(3H)-dione 1 (0.200 g, 1.369 mmol) in ethanol (5 mL) was added 2-hydrazineylquinoline 2 (0.217 g, 1.369 mmol) followed by acetic acid (0.1 mL) at room temperature and reaction mixture was stirred at 60° C. temperature for 16 h. After completion of the reaction (monitored by TLC and LCMS), the reaction mixture was cooled to room temperature and solid precipitate was filtered through Buchner funnel. Dissolved the obtained solid in ethanol (15 mL) and concentrated under reduced pressure to afford 0.070 g (16% yield) of 7-62-51 as yellow oil.

LCMS-Condition-01: [M+H]⁺=287.9; Rₜ=1.728 min.

¹H NMR (400 MHz, DMSO-d₆) δ: 11-33 (br. s, 1H), 8.33 (d, J=7.99 Hz, 1H), 7.89 (d, J=7.99 Hz, 1H), 7.64-7.76 (m, 6H), 7.39-7.50 (m, 2H), 3.93 (s, 2H).

HPLC-Condition-01: 94.50%, Rt: 5.945 min.

### Synthesis of 7-62-53 (VPC 7-84)

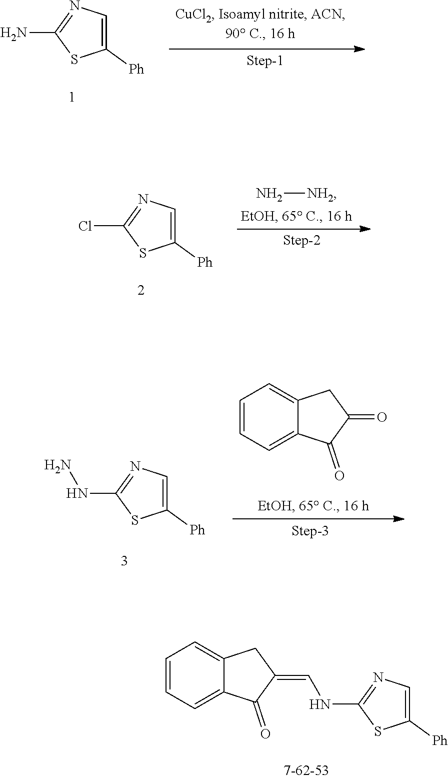

Step-1: Synthesis of 2-chloro-5-phenylthiazole (2):

To a solution of 5-phenylthiazol-2-amine 1 (1.0 g, 5.674 mmol) in acetonitrile (10 mL) was added isoamylnitrite (0.664 g, 5.674 mmol) followed by copper chloride (0.381 g, 2.837 mmol) at room temperature and heated the reaction mixture at 90° C. for 16 h. After completion of the time, the resulting reaction mixture was concentrated under reduced pressure. After completion of the reaction (monitored by TLC and LCMS), the reaction mixture was cool to room temperature, quenched with water (50 mL) and extracted with ethyl acetate (2×25 mL). The combined organic layer was washed with brine (10 mL), dried over anhydrous Na₂SO₄, filtered and concentrated under reduced pressure and the crude compound was purified by Combiflash™ column chromatography eluting with 10-30% ethyl acetate in n-hexane to afford 0.825 g (75% yield) of 2 as an orange solid.

LCMS-Condition-01: [M+H]⁺=196.36; Rₜ=2.48 min

Step-2: Synthesis of 2-hydrazineyl-5-phenylthiazole (3):

To a solution of 2-chloro-5-phenylthiazole 2 (0.820 g, 4.183 mmol) in ethanol (15 mL) was added slowly Hydrazine (535 mL, 16.73 mmol) at room temperature. The reaction mixture was heated at 65° C. temperature and stirred for 16 h. After completion of the reaction, the reaction mixture was cool to room temperature and concentrated under dried under reduced pressure to afford 0.225 g, (28% yield) of 3 as brown solid.

LCMS-Condition-01: [M+H]⁺=192.0; Rₜ=1.022 min.

Step-3: Synthesis of (Z)-2-(2-(5-phenylthiazol-2-yl)hydrazineylidene)-2,3-dihydro-1H-inden-1-one (7-62-53):

To a solution of 2-hydrazineyl-5-phenylthiazole 3 (0.220 g, 1.150 mmol) in ethanol (5 mL) was added 1H-indene-1,2(3H)-dione 4 (0.170 g, 1.150 mmol) at room temperature and heated the reaction mixture at 65° C. for 16 h. After completion of the reaction, the reaction mixture was cool to room temperature and was added (0.1 mL) of acetic acid. The obtained solid was filtered through Buchner funnel and dried under reduced pressure. The crude obtained was purified by Prep-HPLC to afford 0.050 g, (14% yield) of 7-62-53 as Yellow solid.

LCMS-Condition-1: [M+H]⁺=319.95; Rₜ=1.904 min. ¹H NMR (400 MHz, DMSO-d₆) δ: 12.27 (br. s, 1H), 7.80-7.88 (m, 1H), 7.70-7.78 (m, 2H), 7.59-7.69 (m, 3H), 7.38-7.48 (m, 3H), 7.25-7.35 (m, 1H), 3.85 (s, 2H).

HPLC-Condition-03: 92.18%, Rt: 8.209 min.

### Synthesis of 7-62-54

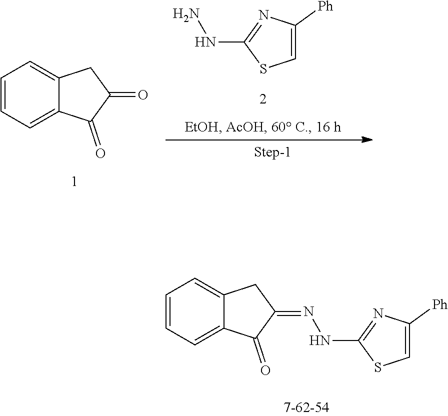 [SMILES: O=C1Cc2ccccc2C1=O ; NNc1nc(-c2ccccc2)cs1 ; O=C1/C(=N\Nc2nc(-c3ccccc3)cs2)Cc2ccccc21]

Step-1: Synthesis of (Z)-2-(2-(4-phenylthiazol-2-yl)hydrazineylidene)-2,3-dihydro-1H-inden-1-one (7-62-54):

To the stirred solution of 1H-indene-1,2(3H)-dione 1 (0.200 g, 1.369 mmol) in ethanol (15 mL) was added 2-hydrazineyl-4-phenylthiazole 2 (0.237, 1.369 mmol) followed by acetic acid (0.1 mL) at room temperature and reaction mixture was stirred at 60° C. temperature for 16 h. After completion of the reaction (monitored by TLC and LCMS), the reaction mixture was cooled to room temperature and solid precipitate was filtered through Buchner funnel. Dissolved the obtained solid in ethanol (15 mL) and concentrated under reduced pressure to afford 0.025 g (7% yield) of 7-62-54 as yellow oil.

LCMS-Condition-01: [M+H]⁺=320.15.05; Rₜ=3.13 min.

¹H NMR (400 MHz, DMSO-d₆) δ: 12.27 (br. s, 1H), 7.91 (d, J=8.31 Hz, 2H), 7.69-7.77 (m, 2H), 7.66 (d, J=8.31 Hz, 1H), 7.42-7.52 (m, 4H), 7.24-7.35 (m, 1H), 3.88 (s, 2H).

HPLC-Condition-03: 95.03%, Rt: 8.427 min.

### Analytical Methods:

¹H and ¹³C NMR spectra (COSY, ¹H/¹³C 2D-correlations) were recorded with Bruker Avance III™ 400 MHz. Processing of the spectra was performed with MestRec™ software and data are reported as follows: chemical shifts (δ) in parts per million, coupling constants (J) in hertz (Hz). The high-resolution mass spectra were recorded in positive ion-mode with an ESI ion source on an Agilent™ Time-of-Flight LC/MS mass spectrometer. HPLC analyses and purity of >95% were performed by analytical reverse-phase HPLC with a Agilent™ instrument with variable detector using column Agilent Zorbax™ 4.6×5 mm, Sum; flow: 2.0 mL·min⁻¹, H₂O (0.1% FA)/CH₃CN (0.1% FA), gradient 2→98% (6 min) and 98% (0.3 min).

A person of skill in the art based on the general knowledge in the art and the information provided herein would be able to synthesize the compounds described herein or modify the compounds described herein.

BRN2 Inhibitor Selection

### Preparation of Reagents

### Preparation of Potassium Phosphate Buffer, 50 mM (pH 7.4)

Potassium phosphate buffer (Kphos) was prepared by adding 0.647 g potassium phosphate monobasic (KH₂PO₄) and 3.527 g potassium phosphate dibasic (KH₂PO₄) to 400 mL of Milli-Q™ water. pH of the buffer was adjusted to 7.4 and volume was made up to 500 mL.

### Preparation of Microsomes

Microsomes (20 mg/mL) were diluted in KH₂PO₄ buffer to prepare a concentration of 0.357 mg/mL.

### Preparation of Test Compounds (BRN2 Inhibitors)

Stock solutions of BRN2 inhibitors were prepared in DMSO at a concentration of 1 mM

### Preparation of NADPH Solution

A stock solution of 3.33 mM NADPH (3.33×) was prepared by dissolving appropriate amount of NADPH in KH₂PO₄ buffer.

### Assay Conditions

Total Incubation volume: 100 μL
Compound concentration: 1 μM
Protein Concentration: 0.25 mg/mL
NADPH: 1 mM
Final DMSO contain: 0.1%
Number of replicates: 2
Time points: 0, 5, 15, 30 and 60 min

### Assay

An 1120 μL aliquot of Kphos buffer (50 mM, pH 7.4) containing liver microsomes (0.357 mg/mL) were added to individual 2 mL tubes (final concentration 0.25 mg/mL). Test compounds (1 mM) and positive controls were directly spiked into respective tubes to prepare a concentration of 1.428 μM (final concentration 1 μM). From the above mix, 70 μL was added to individual wells of 96 well reaction plates and pre-incubated in a 37° C. water bath for 5 min. All the reactions were initiated by adding 30 μL of 3.33 mM NADPH (final concentration 1 mM). Reactions without NADPH and buffer controls (minus NADPH) at 0 min and 60 min were also incubated to rule out non-NADPH metabolism or chemical instability in the incubation buffer. All reactions were terminated using 100 μL of ice-cold acetonitrile containing internal standard at 0, 5, 15, 30 and 60 min. The plates were centrifuged at 4000 RPM for 15 min and 100 μL aliquots were submitted for analysis by LC-MS/MS.

### Bio-Analysis

Samples were monitored for parent compounds disappearance in MRM mode using LC-MS/MS.

The LC-MS/MS conditions and MRM chromatogram.

### Data Analysis

The percent remaining of test compounds and positive control in each sample was determined by considering peak area ratio in the 0 minute sample as 100%. The Half-life of compounds in microsomes is calculated by formula:

Half-life (t½) (min)=0.693/k, where k is gradient of line determined from plot of peak area ratio (compound peak area/internal standard peak area) against time.

In vitro intrinsic clearance (CL'int) (units in mL/min/kg) was calculated using the formula.

$${Cl}_{int}^{′} = \frac{0.693}{in vitro T 1 / 2} \cdot \frac{mL incubation}{mg microsomes} \cdot \frac{45 mg microsomes}{gm liver} \cdot \frac{liver weight in gm *}{Kg b . w}$$

For liver microsomes, scaling factor used was 45 mg microsomal protein per gm liver.
* Indicates liver weight (gm) which varies species wise. For mice the liver weights are 90 gm.

### Cell-Based Testing

### Step 1: Reporter Activity

This assay quantifies inhibition of BRN2 transcriptional activity.
42DE^{NZR} cells stably expressing BRN2-luciferase reporter are plated in a 12-well Corning™ plates at 100,000 cells per well.
Next day cells are treated with 10 and 30 μM of compounds from a stock of 10 mM
24 hours later, cells are washed twice with PBS and lysed in ix Passive Lysis Buffer (Promega™) for 15 minutes.
Plates are frozen at −8° C. and thawed before lysed cells are collected.
Cell lysate is spun at 10,000 rpm for 10 minutes
Supernatant is plated in Flat White Bottom plates and bioluminescence is measured using Tecan M200 Luminometer™.
Bioluminescence readings are normalized to protein concentration of the cell lysate.

Compounds with greater than 75% inhibition at 10 μM with a dose response showing higher inhibition at 30 μM pass this criterion. Decreased expression of luciferase protein is validated via western blot to confirm downregulation and avoid false positive molecules that inhibit luciferase protein activity and not BRN2-drive luciferase expression. Molecules are prioritized by their inhibition potency to measure direct interaction with BRN2 in Step 2.

### Step 2: Drug Affinity Responsive Target Stability (DARTS) Assay

This in cell assay provides a yes/no answer to direct interaction between BRN2 inhibitor and protein.

1) Prepare and place the following on ice:
Pronase
TNC 10×
500 mM Tris-HCl (pH 8.0)
500 mM NaCl, 100 mM CaCl₂
DMSO
Drug
10 mL NP40+Protease inhibitor+Phosphatase inhibitor solution (can store in 4° C. and use for up to 4 weeks):
2 mL NP40 5×
8 mL ddH₂O
1 PhosphoSTOP™ pill
200 uL protease inhibitor 50×

2) Remove the growth medium from the cell plate (42D^{ENZR}) and add 10 mL of cold PBS/vanadate.

3) Scrape the cells. Remove the PBS-cell solution and add in a falcon tube.

4) Name a new 1.5 ml Eppendorf tube and put on ice to pre-chill.

5) Centrifuge the cells at 1500 for 5 minutes at 4° C. Remove supernatant and re-suspend the cells in 600 uL NP40 lysis buffer by pipetting up and down. If the cell pellet is small use less volume, for example 300 μl)

6) Transfer the lysing cells into the pre-chilled Eppendorf tube. Incubate on ice for 20 minutes. If not continuing the DARTS on the same day, you can put the lysate in −30 and work on it later.

### Preparing Cell Lysates for DARTS:

7) Centrifuge the tube at maximum speed for 25-30 minutes at 4° C.

8) Discard the pellet (either transfer the supernatant in a new tube or just take out the pellet).

9) Measure protein concentration of the lysates using the BCA.

*Optimal protein concentration for DARTS is between 4-6 μg/μL. A little higher or lower works too (roughly 3 to 8).

10) Split the lysates into two samples by transferring half of the liquid into each of two tubes.

Warm the lysates to room temperature.

Incubate Protein Lysates with Small Molecule:

11) Add into one tube 3 μL DMSO, and into the other tube 3 μL small molecule with 10 mM final concentration.

12) Mix the samples immediately by gently flicking the tubes, spin the tubes for the drops, and allow them to incubate at room temperature for 1 hour. 45 mins in, start preparing the Pronase™.

13) Thaw one aliquot of 10 mg/mL Pronase™ (protease) and place on ice. (to make new Pronase™ stock 10 mg/ml)

14) Thaw one aliquot of TNC 10×, dilute to 1×(add 100 μL to 900 μL H₂O). Don't Freeze and thaw 10×TNC.

15) Dilute Pronase™ to 1.25 mg/mL by mixing 12.5 μL Pronase™ with 87.5 μL cold 1×TNC Buffer, which will serve as the 1:100 Pronase™ stock solution.

16) Dilute the 1:100 Pronase™ solution serially by mixing with 1×TNC to create 1:100, 1:500, 1:1000, 1:5000, and 1:10000 Pronase™ stock solutions. MIX every tube well by flicking before making the next solution.
1:500 . . . Take 20 μL of 1:100 and add 80 ul of 1×TNC
1:1000 . . . Take 50 μL of 1:500 and add 50 ul of 1×TNC
1:5000 . . . Take 20 μL of 1:1000 and add 80 ul of 1×TNC
1:10000 . . . Take 50 μL of 1:5000 and add 50 ul of 1×TNC

### Performing Proteolysis:

17) Prepare 5 aliquots from each of the tubes, each with 30 uL, and save the remaining of each sample to be used as a control sample (if previously you used less than 600 μl of NP40 in step 5, you might want to omit some pronase dilutions).

18) Add 2 μL of 1:100 pronase to one drug-treated aliquot and to one aliquot of DMSO sample. Mix them well by flicking and then incubate at room temperature. Continue adding 2 μL of the corresponding pronase stock solution into each of the remaining aliquots.

19) After 30 minutes, add 10 uL of 4× sample buffer into each sample. Heat at 100° C. for 5 minutes. Spin down to collect the drops.

20) The samples are ready for Western blot gel running. You can run them now, or freeze to run later (in that case, after thawing you have to vortex and spin down, then run). For primary antibody, use your protein of interest (here BRN2 in Rb) and loading control proteins like GAPDH, actin, or Vinculin (better if its Ms antibody).

### Molecules Displaying Positive Binding Progress to Step 3.

### Step 3: Cell Proliferation

This step selects BRN2 inhibitors to inhibit proliferation of BRN2 expressing 42DE^{N}ZR cells while being completely ineffective against 16D^{CRPC} cells, which do not express BRN2.
1. Plate 42DE^{N}ZR (˜7000 cells per well) and 16D^{CRPC} (˜5000 cells per well) cells in 96 well plate with a volume of 100 μL per well.
2. Next day 1 mL aliquots of media are combined with 3× the desired concentration of compounds. The aliquots are vortexed and 50 μL of each 3× concentration is added to the 100 μL of media in each well to give the ix desired concentration.

The following are the desired concentrations:

0, 10 nM, 100 nM, 500 nM, 1 μM, 2.5 μM, 5 μM, 10 μM, 15 μM, 20 μM, 30 μM.
3. Approximately 72 hours later, 17 μL of 10% Glutaraldehyde is added to each well and plates are placed on a shaker for 20-30 minutes.
4. Media is washed off the fixed cells by slow submersion in a container of water. Then the water is removed by tapping the plate upside down.
5. 75 μL of Crystal Violet™ solution is added into the 96 well plates and placed on a shaker for ˜30 minutes.
6. Media is washed off the fixed cells by repeated slow submersion in a container of water and the plates are allowed to dry over-night.
7. Next day, 75 μL of Sorensen Solution is accurately pipetted into each well. The plates placed on a shaker for ˜30 minutes.
8. Absorbance is measured in the plate at 590 nm.
   Compounds that Show Specificity Towards 42DE^{N}ZR Cells Vs 16D^{CRPC} Cells Progress to Step 4.

### Step 4: Suppression of Target Genes

This step evaluates the ability of BRN2 inhibitors to suppress expression of BRN2 target genes NCAM1 and SOX2, as well as NEPC marker CHGA.
1. 42D^{ENZR} cells are plated in 10 cm cell culture plates at 1,000,000 cells per plate.
2. The next day, cells are treated with compounds at 0, 1, 5 and 10 PM.
3. Approximately 48 hours later, cells are washed once with PBS and mRNA is extracted using PureLink RNA Mini Kit™ (Thermo Fisher™) as per manufacturer's instructions.
4. cDNA is prepared using SuperScript IV™ (Thermo Fisher™) as per manufacturer's instructions.
5. Using Tagman™ Probe master mix (Roche™) and Tagman™ Primers (Thermo Fisher™) q-PCR is conducted on the samples according to the following protocol: For each sample prepare a small Eppendorf™ tube containing:

| | | | |
|---|---|---|---|
| 2X Taqman ™ Mix | 5 μl | ×5 | 62 μl |
| cDNA from RT reaction - sample | 1 μl | × | μl |
| DEPC water | 1 μl | × | μl |
| Final volume in each well | 7 μl | | |

For Each Gene (e.g. Probe and rRNA) Prepare MasterMix:

| | 1 sample | replicates |
|---|---|---|
| 20X primer mix | 0.5 μl | 5 μl |
| H₂O | 2.5 μl | μl |
| Put in each well | 3.0 μl | |

Plates are run on ABI ViiA7 machine with standard Tagman™ protocol. Cycle times are analyzed via ΔΔCT method. Relative expression of target genes NCAM1 and SOX2 as well as CHGA are quantified compared to control. Compounds clear this final selection step if the expression of these genes is reduced in a dose dependent manner.

### EXAMPLES

### Example m—Compound Testing

In TABLE 1 compounds were tested for metabolic stability % and T½ (MLM). Some compounds were also tested to determine their specificity to 42D and not 16D cells and BLI binding as shown in TABLE 1.

TABLE 1
Structural and experimental data for the BRN2 interactors.

| | | Met Stab | | | |
|---|---|---|---|---|---|
| | | % (60 | T1/2 in | | |
| | | mins) | mins | BLI | Growth |
| VPC-ID | Structure | MLM | (MLM) | binding | Specificity |
| VPC-7 | 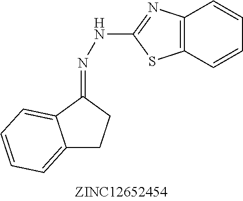 [SMILES: c1ccc2c(c1)CC/C2=N\Nc1nc2ccccc2s1] | | | | 42D not 16D |
| VPC-8 | 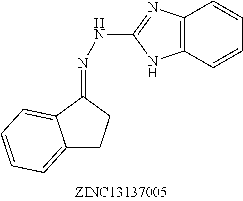 [SMILES: c1ccc2c(c1)CC/C2=N\Nc1nc2ccccc2[nH]1] | | | | 42D and 16D non-specific |
| VPC-39 | 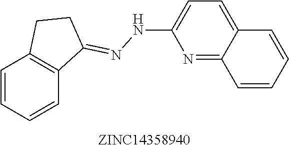 [SMILES: c1ccc2c(c1)CC/C2=N\Nc1ccc2ccccc2n1] | | | | non-specific |
| 7-62 | 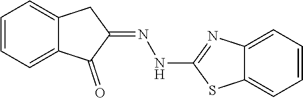 [SMILES: O=C1/C(=N\Nc2nc3ccccc3s2)Cc2ccccc21] | 31 | 40 | Strong binding observed using DARTS assay | Specific to 42D (IC50 is 1.0 μM) and no effect on 16D. |
| 7-91 | 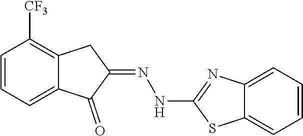 [SMILES: O=C1/C(=N\Nc2nc3ccccc3s2)Cc2c1cccc2C(F)(F)F] | 67 | >60 | Positive binding | Specific to 42D IC50 ~2 μM |
| 7-106 | 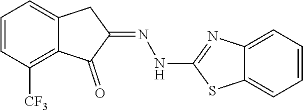 [SMILES: O=C1/C(=N\Nc2nc3ccccc3s2)Cc2cccc(C(F)(F)F)c21] | 38 | 46 | Positive binding | Specific to 42D cells (IC50 is about 2 μM) |
| 7-94 | 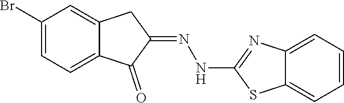 [SMILES: O=C1/C(=N\Nc2nc3ccccc3s2)Cc2cc(Br)ccc21] | 50 | >60 | Positive binding | Specific to 42D IC50 ~2 μM |
| 7-97 | 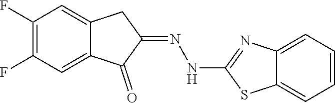 [SMILES: O=C1/C(=N\Nc2nc3ccccc3s2)Cc2cc(F)c(F)cc21] | 69 | >60 | Positive binding | Specific to 42D IC50 ~2 μM |
| 7-98 | 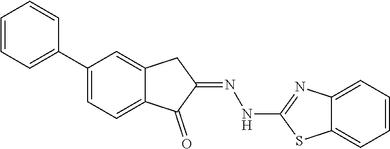 [SMILES: O=C1/C(=N\Nc2nc3ccccc3s2)Cc2cc(-c3ccccc3)ccc21] | 26 | 33 | | Specific to 42D IC50 ~2 μM |
| 7-100 | 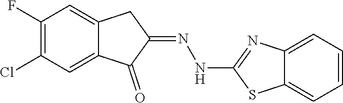 [SMILES: O=C1/C(=N\Nc2nc3ccccc3s2)Cc2cc(F)c(Cl)cc21] | 30 | 38 | Positive binding | Specific to 42D IC50 ~2 μM |
| 7-101 | 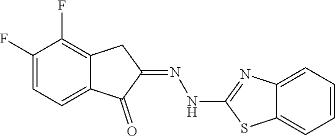 [SMILES: O=C1/C(=N\Nc2nc3ccccc3s2)Cc2c1ccc(F)c2F] | 67 | >60 | Positive binding | Specific to 42D cells (IC50 is 1- 2.5 μM) |
| 7-102 | 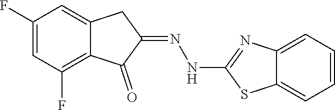 [SMILES: O=C1/C(=N\Nc2nc3ccccc3s2)Cc2cc(F)cc(F)c21] | 46 | 56 | Positive binding but not strong | Specific to 42D cells (IC50 is about 2.5 μM) |
| 7-50 | 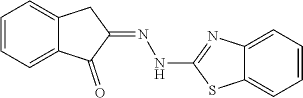 [SMILES: O=C1/C(=N\Nc2nc3ccccc3s2)Cc2ccccc21] | 9 | 25 | Positive | Specific to 42D (IC50 is 3 μM) and no effect on 16D. |
| 7-70 | 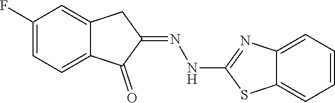 [SMILES: O=C1/C(=N\Nc2nc3ccccc3s2)Cc2cc(F)ccc21] | 38 | 50 | Positive binding | Specific to 42D with IC50 ~5 μM |
| 7-81 | 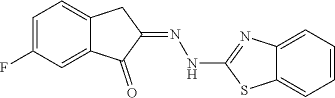 [SMILES: O=C1/C(=N\Nc2nc3ccccc3s2)Cc2ccc(F)cc21] | 26 | 22 | Positive binding | Specific to 42D |
| 7-125 | 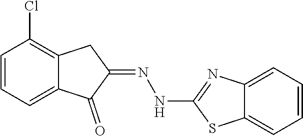 [SMILES: O=C1/C(=N\Nc2nc3ccccc3s2)Cc2c(Cl)cccc21] | 36 | 44 | No binding | specific |
| 7-126 | 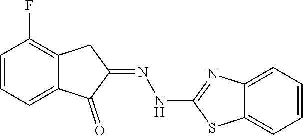 [SMILES: O=C1/C(=N\Nc2nc3ccccc3s2)Cc2c(F)cccc21] | 6 | 15 | binding | specific |
| 7-80 | 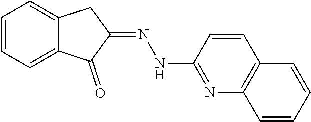 [SMILES: O=C1/C(=N\Nc2ccc3ccccc3n2)Cc2ccccc21] | 4 | 10 | | Specific to 42D |
| 7-57 | 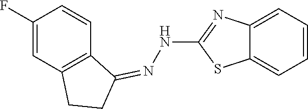 [SMILES: Fc1ccc2c(c1)CC/C2=N/Nc1nc2ccccc2s1] | 0 | 8 | Positive | Specific to 42D (IC50 is 10 μM) and no effect on 16D. |
| 7-65 | 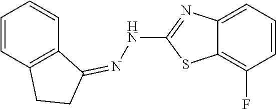 [SMILES: Fc1cccc2nc(N/N=C3/CCc4ccccc43)sc12] | 0 | 6 | | Specific to 42D (IC50 is 10 μM) and no effect on 16D. |
| 7-66 | 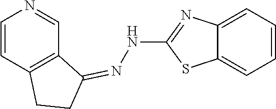 [SMILES: c1ccc2sc(N/N=C3/CCc4ccncc43)nc2c1] | 1 | 8 | | Specific to 42D (IC50 is 10 μM) and no effect on 16D. |
| 7-92 | 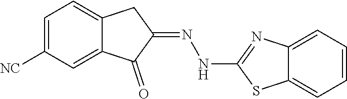 [SMILES: N#Cc1ccc2c(c1)C(=O)/C(=N\Nc1nc3ccccc3s1)C2] | 41 | 48 | Positive binding | Specific but weak, 42D IC50 is ~15 μM |
| 7-93 | 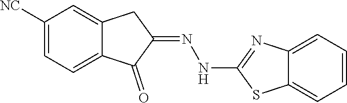 [SMILES: N#Cc1ccc2c(c1)C/C(=N/Nc1nc3ccccc3s1)C2=O] | 39 | 44 | no binding | Specific but weak, 42D IC50 is ~15 μM |
| 7-95 | 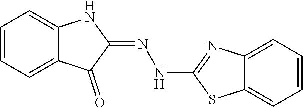 [SMILES: O=C1/C(=N\Nc2nc3ccccc3s2)Nc2ccccc21] | 69 | >60 | Positive binding | Specific but weak, 42D IC50 is ~15 μM |
| 7-96 | 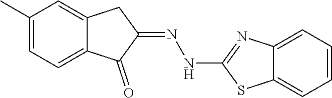 [SMILES: Cc1ccc2c(c1)C/C(=N/Nc1nc3ccccc3s1)C2=O] | 22 | 31 | inconclusive, needs to be repeated | Specific but weak, 42D IC50 is ~15 μM |
| 7-71 | 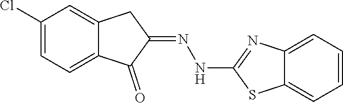 [SMILES: O=C1/C(=N\Nc2nc3ccccc3s2)Cc2cc(Cl)ccc21] | 35 | 47 | Positive binding | Specific to 42D but weak with IC50 ~15 μM |
| 7-72 | 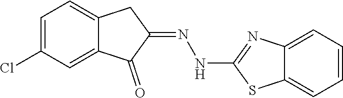 [SMILES: O=C1/C(=N\Nc2nc3ccccc3s2)Cc2ccc(Cl)cc21] | 8 | 17 | | Specific to 42D at low doses, affects 16D >15 μM |
| 7-73 | 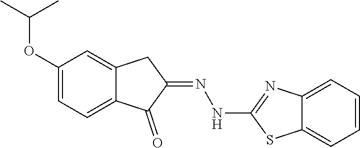 [SMILES: CC(C)Oc1ccc2c(c1)C/C(=N/Nc1nc3ccccc3s1)C2=O] | 19 | 26 | Positive binding | Specific to 42D at low doses, affects 16D >15 μM |
| 7-108 | 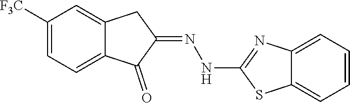 [SMILES: O=C1/C(=N\Nc2nc3ccccc3s2)Cc2cc(C(F)(F)F)ccc21] | 68 | >60 | Positive binding | Specific to 42D cells until 10 μM and activity on 16D >15 μM |
| 7-74 | 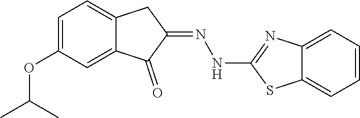 [SMILES: CC(C)Oc1ccc2c(c1)C(=O)/C(=N\Nc1nc3ccccc3s1)C2] | 6 | 17 | Positive binding | Specific to 42D at low doses, affects 16D >20 μM |
| 7-79 | 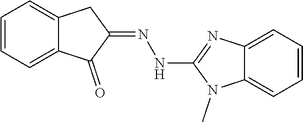 [SMILES: Cn1c(N/N=C2/Cc3ccccc3C2=O)nc2ccccc21] | 2 | 6 | | Specific but weak, 42D IC50 is >15 μM |
| 7-36 | 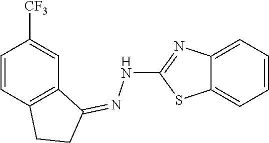 [SMILES: FC(F)(F)c1ccc2c(c1)/C(=N\Nc1nc3ccccc3s1)CC2] | 78 | >60 | DARTS- good binding | Specific to 42D, but weak activity |
| 7-52 | 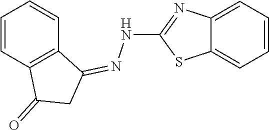 [SMILES: O=C1C/C(=N/Nc2nc3ccccc3s2)c2ccccc21] | 9 | 23 | Positive | Potent (IC50 is 3-7 μM) but effective on both 42D and 16D cells |
| 7-53 | 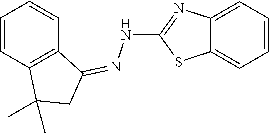 [SMILES: CC1(C)C/C(=N/Nc2nc3ccccc3s2)c2ccccc21] | 4 | 13 | | Potent (IC50 is 10 μM) but effective on both 42D and 16D cells |
| 7-56 | 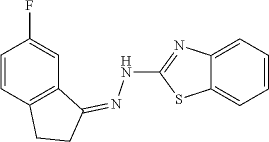 [SMILES: Fc1ccc2c(c1)/C(=N\Nc1nc3ccccc3s1)CC2] | 0 | 8 | | Potent (IC50 is 10 μM) but effective on both 42D and 16D cells |
| 7-58 | 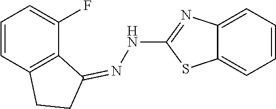 [SMILES: Fc1cccc2c1/C(=N\Nc1nc3ccccc3s1)CC2] | 0 | 7 | | Potent (IC50 is 10 μM) but effective on both 42D and 16D cells |
| 7-61 | 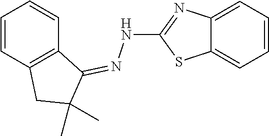 [SMILES: CC1(C)Cc2ccccc2/C1=N\Nc1nc2ccccc2s1] | 1 | 10 | | Potent (IC50 is 10 μM) but effective on both 42D and 16D cells |
| 7-99 | 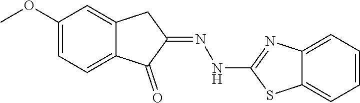 [SMILES: COc1ccc2c(c1)C/C(=N/Nc1nc3ccccc3s1)C2=O] | 6 | 15 | | Potent but effective on both 42D and 16D cells |
| 7-107 | 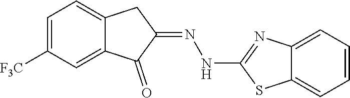 [SMILES: O=C1/C(=N\Nc2nc3ccccc3s2)Cc2ccc(C(F)(F)F)cc21] | 82 | >60 | | Low potency |
| 7-30 | 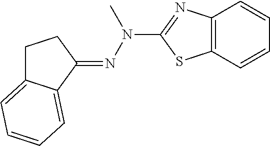 [SMILES: CN(/N=C1\CCc2ccccc21)c1nc2ccccc2s1] | 1 | 11 | | Non-specific. Effective on both 42D and 16D cells |
| 7-31 | 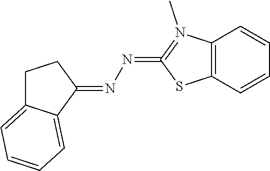 | 15 | 32 | | Non-specific. Effective on both 42D and 16D cells |
| 7-32 | 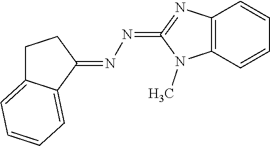 | 0 | NC | | Non-specific. Effective on both 42D and 16D cells |
| 7-33 | 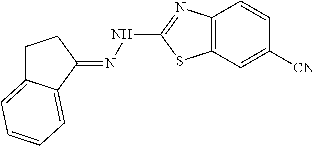 [SMILES: N#Cc1ccc2nc(N/N=C3\CCc4ccccc43)sc2c1] | 35 | 47 | | Non-specific. Effective on both 42D and 16D cells |
| 7-34 | 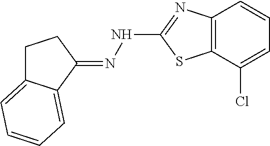 [SMILES: Clc1cccc2nc(N/N=C3\CCc4ccccc43)sc12] | 0 | 6 | | Non-specific. Effective on both 42D or 16D cells |
| 7-35 | 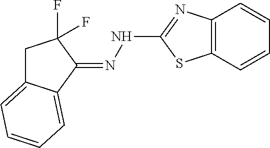 [SMILES: FC1(F)Cc2ccccc2/C1=N/Nc1nc2ccccc2s1] | 1 | 12 | | Not effective on 42D or 16D cells |
| 7-37 | 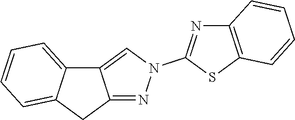 [SMILES: c1ccc2c(c1)Cc1nn(-c3nc4ccccc4s3)cc1-2] | 7 | 16 | | Not effective on 42D or 16D cells |
| 7-38 | 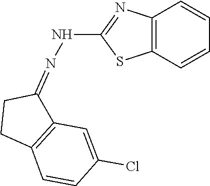 [SMILES: Clc1ccc2c(c1)/C(=N\Nc1nc3ccccc3s1)CC2] | 1 | 8 | | Not effective on 42D or 16D cells |
| 7-39 | 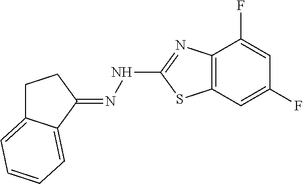 [SMILES: Fc1cc(F)c2nc(N/N=C3\CCc4ccccc43)sc2c1] | 7 | 17 | | Not effective on 42D or 16D cells |
| 7-40 | 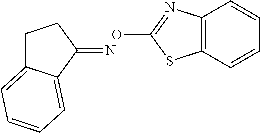 | 0 | NC | | Not effective on 42D or 16D cells |
| 7-41 | 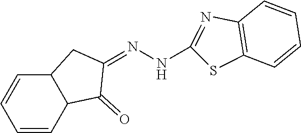 [SMILES: O=C1/C(=N\Nc2nc3ccccc3s2)CC2C=CC=CC12] | 28 | 33 | | Not effective on 42D or 16D cells |
| 7-42 | 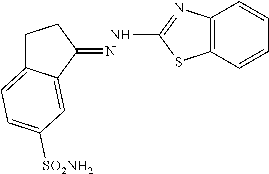 | 49 | 60 | | Not effective on 42D or 16D cells |
| 7-43 | 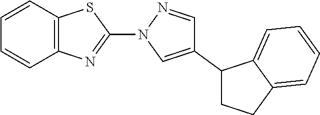 [SMILES: c1ccc2c(c1)CCC2c1cnn(-c2nc3ccccc3s2)c1] | 1 | 10 | | Not effective on 42D or 16D cells |
| 7-44 | 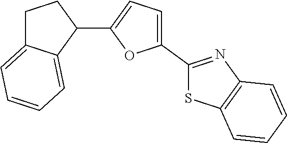 [SMILES: c1ccc2c(c1)CCC2c1ccc(-c2nc3ccccc3s2)o1] | 1 | 10 | | Not effective on 42D or 16D cells |
| 7-45 | 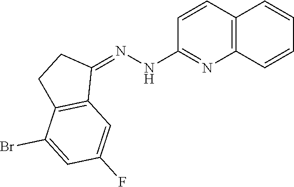 [SMILES: Fc1cc(Br)c2c(c1)/C(=N\Nc1ccc3ccccc3n1)CC2] | 14 | 21 | | Not effective on 42D or 16D cells |
| 7-46 | 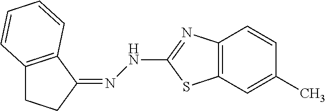 [SMILES: Cc1ccc2nc(N/N=C3/CCc4ccccc43)sc2c1] | 5 | 13 | | Not effective on 42D or 16D cells |
| 7-47 | 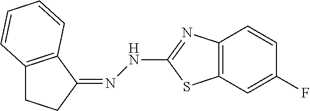 [SMILES: Fc1ccc2nc(N/N=C3/CCc4ccccc43)sc2c1] | 0 | 7 | | Not effective on 42D or 16D cells |
| 7-48 | 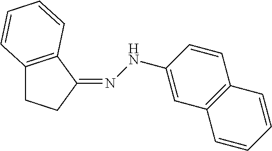 [SMILES: c1ccc2c(c1)CC/C2=N/Nc1ccc2ccccc2c1] | 14 | 22 | | Not effective on 42D or 16D cells |
| 7-49 | 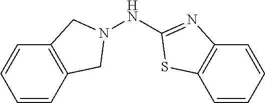 [SMILES: c1ccc2c(c1)CN(Nc1nc3ccccc3s1)C2] | 1 | 14 | | Not effective on 42D or 16D cells |
| 7-51 | 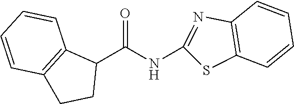 [SMILES: O=C(Nc1nc2ccccc2s1)C1CCc2ccccc21] | 1 | 12 | | Not effective on 42D or 16D cells |
| 7-54 | 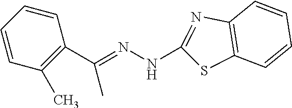 [SMILES: C/C(=N\Nc1nc2ccccc2s1)c1ccccc1C] | 0 | nc | | Not effective on 42D or 16D cells |
| 7-60 | 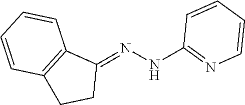 [SMILES: c1ccc(N/N=C2\CCc3ccccc32)nc1] | 0 | 8 | | Not effective on 42D or 16D cells |
| 7-63 | 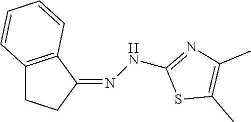 [SMILES: Cc1nc(N/N=C2/CCc3ccccc32)sc1C] | 1 | 11 | | Not effective on 42D or 16D cells |
| 7-64 | 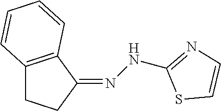 [SMILES: c1ccc2c(c1)CC/C2=N/Nc1nccs1] | 0 | 8 | | Not effective on 42D or 16D cells |
| 7-67 | 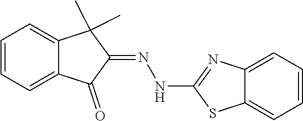 [SMILES: CC1(C)/C(=N/Nc2nc3ccccc3s2)C(=O)c2ccccc21] | 28 | 34 | | not specific |
| 7-68 | 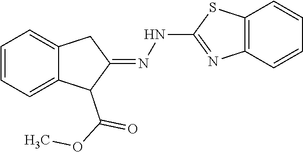 [SMILES: COC(=O)C1/C(=N/Nc2nc3ccccc3s2)Cc2ccccc21] | 0 | <5 | | not specific |
| 7-69 | 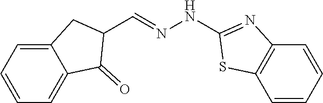 [SMILES: O=C1c2ccccc2CC1/C=N/Nc1nc2ccccc2s1] | 33 | 44 | | not specific |
| 7-76 | 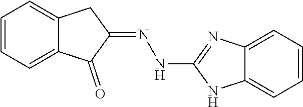 [SMILES: O=C1/C(=N\Nc2nc3ccccc3[nH]2)Cc2ccccc21] | 1 | 10 | | Not effective on 42D or 16D cells |
| 7-77 | 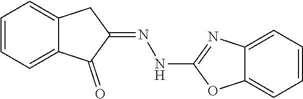 [SMILES: O=C1/C(=N\Nc2nc3ccccc3o2)Cc2ccccc21] | 8 | 16 | | Not specific |
| 7-78 | 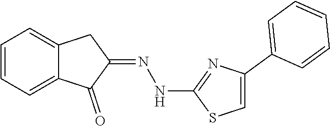 [SMILES: O=C1/C(=N\Nc2nc(-c3ccccc3)cs2)Cc2ccccc21] | 32 | 32 | | Not effective on 42D or 16D cells |
| 7-82 | 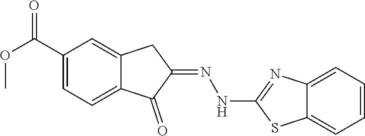 [SMILES: COC(=O)c1ccc2c(c1)C/C(=N/Nc1nc3ccccc3s1)C2=O] | 63 | >60 | | Not effective on 42D or 16D cells |
| 7-110 | 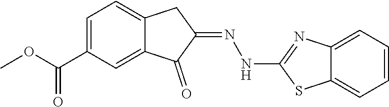 [SMILES: COC(=O)c1ccc2c(c1)C(=O)/C(=N\Nc1nc3ccccc3s1)C2] | 25 | 32 | No binding | No activity |
| 7-83 | 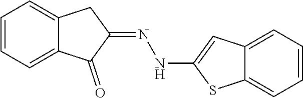 [SMILES: O=C1/C(=N\Nc2cc3ccccc3s2)Cc2ccccc21] | 16 | 17 | | Not effective on 42D or 16D cells |
| 7-84 | 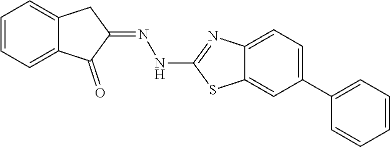 [SMILES: O=C1/C(=N\Nc2nc3ccc(-c4ccccc4)cc3s2)Cc2ccccc21] | 8 | 21 | | Not specific |
| 7-85 | 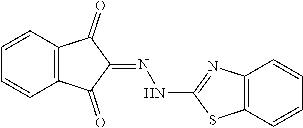 [SMILES: O=c1c(=NNc2nc3ccccc3s2)c(=O)c2ccccc12] | 25 | 32 | | Not specific |
| 7-86 | 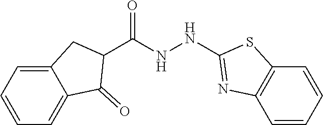 [SMILES: O=C(NNc1nc2ccccc2s1)C1Cc2ccccc2C1=O] | 18 | 19 | | Not specific |
| 7-87 | 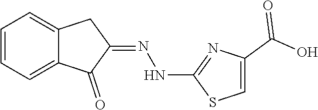 [SMILES: O=C(O)c1csc(N/N=C2/Cc3ccccc3C2=O)n1] | 98 | >60 | | Not specific |
| 7-88 | 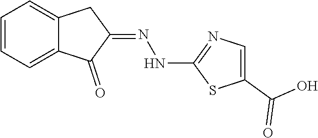 [SMILES: O=C(O)c1cnc(N/N=C2/Cc3ccccc3C2=O)s1] | 94 | >60 | | Not specific |
| 7-89 | 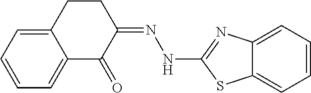 [SMILES: O=C1/C(=N\Nc2nc3ccccc3s2)CCc2ccccc21] | 2 | 11 | | Not specific |
| 7-90 | 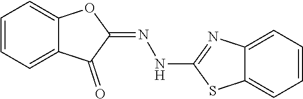 [SMILES: O=C1/C(=N\Nc2nc3ccccc3s2)Oc2ccccc21] | 4 | 13 | no binding | Not specific |
| 7-103 | 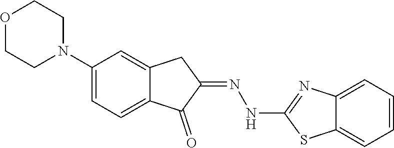 [SMILES: O=C1/C(=N\Nc2nc3ccccc3s2)Cc2cc(N3CCOCC3)ccc21] | 42 | 44 | No binding | Not specific |
| 7-104 | 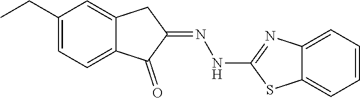 [SMILES: CCc1ccc2c(c1)C/C(=N/Nc1nc3ccccc3s1)C2=O] | 17 | 25 | No binding | Not specific |
| 7-105 | 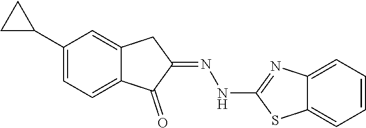 [SMILES: O=C1/C(=N\Nc2nc3ccccc3s2)Cc2cc(C3CC3)ccc21] | 75 | >60 | No binding | Not specific |
| 7-109 | 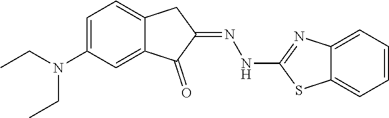 [SMILES: CCN(CC)c1ccc2c(c1)C(=O)/C(=N\Nc1nc3ccccc3s1)C2] | 3 | 13 | Positive binding | Not specific |
| 7-111 | 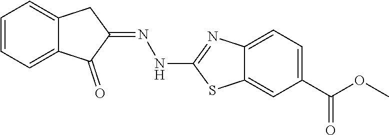 [SMILES: COC(=O)c1ccc2nc(N/N=C3/Cc4ccccc4C3=O)sc2c1] | 6 | 15 | No binding | No activity |
| 7-112 | 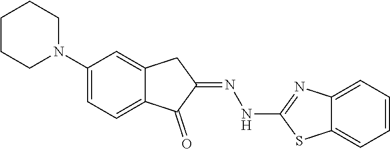 [SMILES: O=C1/C(=N\Nc2nc3ccccc3s2)Cc2cc(N3CCCCC3)ccc21] | 26 | 32 | No binding | Not specific |
| 7-113 | 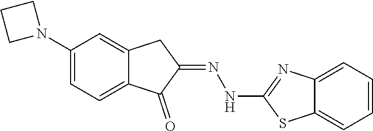 [SMILES: O=C1/C(=N\Nc2nc3ccccc3s2)Cc2cc(N3CCC3)ccc21] | 6 | 15 | No binding | precipitates |
| 7-114 | 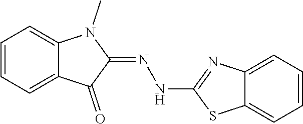 [SMILES: CN1/C(=N/Nc2nc3ccccc3s2)C(=O)c2ccccc21] | 40 | 44 | No binding | precipitates |
| 7-115 | 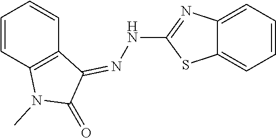 [SMILES: CN1C(=O)/C(=N/Nc2nc3ccccc3s2)c2ccccc21] | 2 | 7 | No binding | Not specific |
| 7-116 | 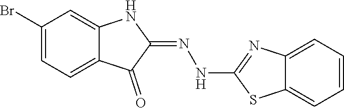 [SMILES: O=C1/C(=N\Nc2nc3ccccc3s2)Nc2cc(Br)ccc21] | 64 | >60 | No binding | Not specific |
| 7-117 | 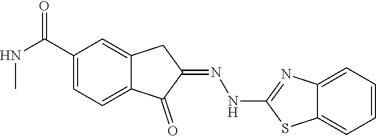 [SMILES: CNC(=O)c1ccc2c(c1)C/C(=N/Nc1nc3ccccc3s1)C2=O] | | | No binding | Not specific |
| 7-118 | 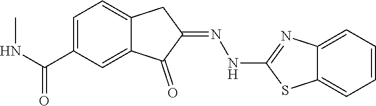 [SMILES: CNC(=O)c1ccc2c(c1)C(=O)/C(=N\Nc1nc3ccccc3s1)C2] | | | No binding | very low activity |
| 7-119 | 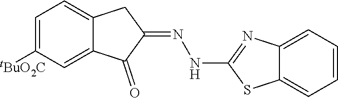 [SMILES: CC(C)(C)OC(=O)c1ccc2c(c1)C(=O)/C(=N\Nc1nc3ccccc3s1)C2] | 18 | 27 | No binding | Not specific |
| 7-120 | 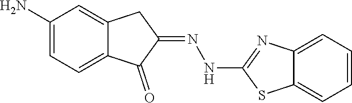 [SMILES: Nc1ccc2c(c1)C/C(=N/Nc1nc3ccccc3s1)C2=O] | 17 | 24 | No binding | Not specific |
| 7-121 | 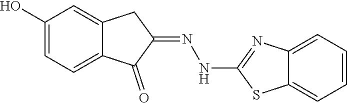 [SMILES: O=C1/C(=N\Nc2nc3ccccc3s2)Cc2cc(O)ccc21] | 30 | 36 | Positive binding | Not specific |
| 7-122 | 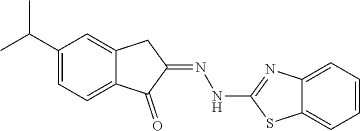 [SMILES: CC(C)c1ccc2c(c1)C/C(=N/Nc1nc3ccccc3s1)C2=O] | 1 | 10 | No binding | Not specific |
| 7-123 | 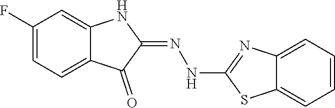 [SMILES: O=C1/C(=N\Nc2nc3ccccc3s2)Nc2cc(F)ccc21] | 18 | 25 | No binding | Not specific |
| 7-124 | 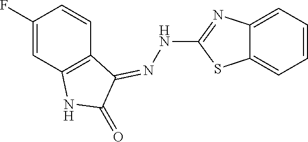 [SMILES: O=C1Nc2cc(F)ccc2/C1=N\Nc1nc2ccccc2s1] | 21 | 27 | No binding | Not specific |
| 7-127 | 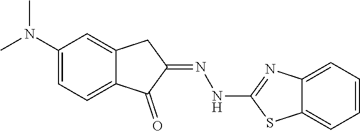 [SMILES: CN(C)c1ccc2c(c1)C/C(=N/Nc1nc3ccccc3s1)C2=O] | 14 | 23 | No binding | Not specific |

VPC-7 showed 82.14% BRN2 inhibition at 10 μM and 97.76% BRN2 inhibition at 30 μM. VPC-8 showed 84.50% BRN2 inhibition at 10 μM and 96.06% BRN2 inhibition at 30 μM. VPC-39 showed 99.05% BRN2 inhibition at 10 μM and 99.92% BRN2 inhibition at 30 μM.

TABLE 2
List of BRN2 Inhibitory Compounds with Specificity

| | | Met Stab | | | |
|---|---|---|---|---|---|
| | | % (60 | T1/2 in | | |
| | | mins) | mins | | Growth |
| VPC-ID | Structure | MLM | (MLM) | BLI binding | Specificity |
| VPC-7 | 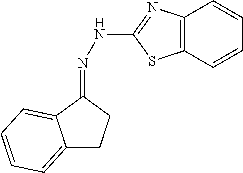 [SMILES: c1ccc2c(c1)CC/C2=N\Nc1nc2ccccc2s1] | | | | 42D not 16D |
| 7-62 | 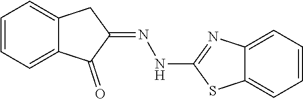 [SMILES: O=C1/C(=N\Nc2nc3ccccc3s2)Cc2ccccc21] | 31 | 40 | Strong binding observed using DARTS assay | Specific to 42D (IC50 is 1.0 μM) and no effect on 16D. |
| 7-91 | 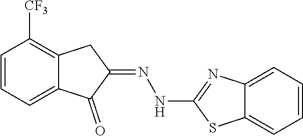 [SMILES: O=C1/C(=N\Nc2nc3ccccc3s2)Cc2c1cccc2C(F)(F)F] | 67 | >60 | Positive binding | Specific to 42D IC50 ~2 μM |
| 7-106 | 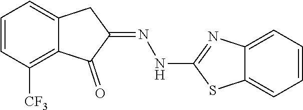 [SMILES: O=C1/C(=N\Nc2nc3ccccc3s2)Cc2cccc(C(F)(F)F)c21] | 38 | 46 | Positive binding | Specific to 42D cells (IC50 is about 2 μM) |
| 7-94 | 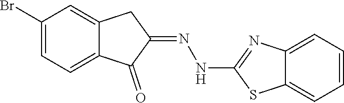 [SMILES: O=C1/C(=N\Nc2nc3ccccc3s2)Cc2cc(Br)ccc21] | 50 | >60 | Positive binding | Specific to 42D IC50 ~2 μM |
| 7-97 | 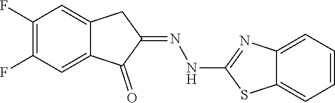 [SMILES: O=C1/C(=N\Nc2nc3ccccc3s2)Cc2cc(F)c(F)cc21] | 69 | >60 | Positive binding | Specific to 42D IC50 ~2 μM |
| 7-101 | 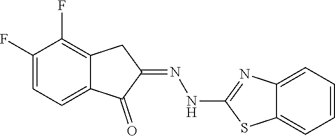 [SMILES: O=C1/C(=N\Nc2nc3ccccc3s2)Cc2c1ccc(F)c2F] | 67 | >60 | Positive binding | Specific to 42D cells (IC50 is 1- 2.5 μM) |
| 7-102 | 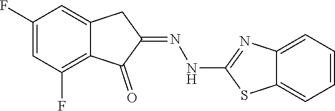 [SMILES: O=C1/C(=N\Nc2nc3ccccc3s2)Cc2cc(F)cc(F)c21] | 46 | 56 | Positive binding but not strong | Specific to 42D cells (IC50 is about 2.5 μM) |
| 7-98 | 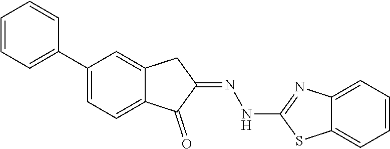 [SMILES: O=C1/C(=N\Nc2nc3ccccc3s2)Cc2cc(-c3ccccc3)ccc21] | 26 | 33 | | Specific to 42D IC50 ~2 μM |
| 7-100 | 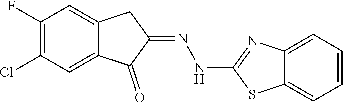 [SMILES: O=C1/C(=N\Nc2nc3ccccc3s2)Cc2cc(F)c(Cl)cc21] | 30 | 38 | Positive binding | Specific to 42D IC50 ~2 μM |
| 7-50 | 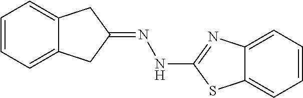 [SMILES: c1ccc2c(c1)CC(=NNc1nc3ccccc3s1)C2] | 9 | 25 | Positive | Specific to 42D (IC50 is 3 μM) and no effect on 16D. |
| 7-70 | 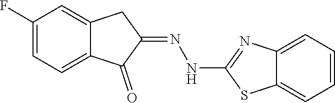 [SMILES: O=C1/C(=N\Nc2nc3ccccc3s2)Cc2cc(F)ccc21] | 38 | 50 | Positive binding | Specific to 42D with IC50 ~5 μM |
| 7-81. | 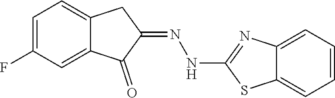 [SMILES: O=C1/C(=N\Nc2nc3ccccc3s2)Cc2ccc(F)cc21] | 26 | 22 | Positive binding | Specific to 42D |
| 7-125 | 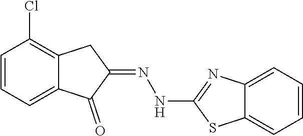 [SMILES: O=C1/C(=N\Nc2nc3ccccc3s2)Cc2c(Cl)cccc21] | 36 | 44 | No binding | specific |
| 7-126 | 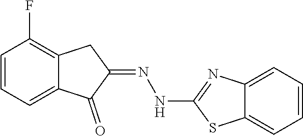 [SMILES: O=C1/C(=N\Nc2nc3ccccc3s2)Cc2c(F)cccc21] | 6 | 15 | binding | specific |
| 7-80 | 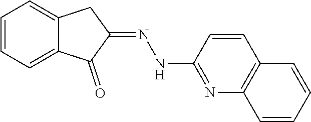 [SMILES: O=C1/C(=N\Nc2ccc3ccccc3n2)Cc2ccccc21] | 4 | 10 | | Specific to 42D |
| 7-57 | 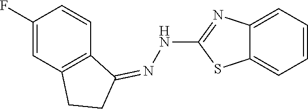 [SMILES: Fc1ccc2c(c1)CC/C2=N/Nc1nc2ccccc2s1] | 0 | 8 | Positive | Specific to 42D (IC50 is 10 μM) |
| 7-65 | 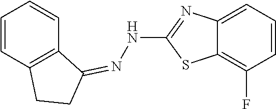 [SMILES: Fc1cccc2nc(N/N=C3/CCc4ccccc43)sc12] | 0 | 6 | | Specific to 42D (IC50 is 10 μM) |
| 7-66 | 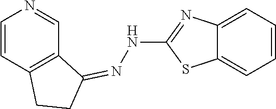 [SMILES: c1ccc2sc(N/N=C3/CCc4ccncc43)nc2c1] | 1 | 8 | | Specific to 42D (IC50 is 10 μM) |
| 7-92 | 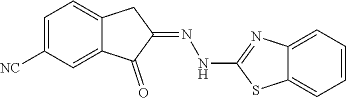 [SMILES: N#Cc1ccc2c(c1)C(=O)/C(=N\Nc1nc3ccccc3s1)C2] | 41 | 48 | Positive binding | Specific but weak, 42D IC50 is ~15 μM |
| 7-93 | 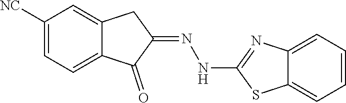 [SMILES: N#Cc1ccc2c(c1)C/C(=N/Nc1nc3ccccc3s1)C2=O] | 39 | 44 | no binding | Specific but weak, 42D IC50 is ~15 μM |
| 7-95 | 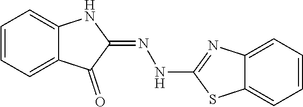 [SMILES: O=C1/C(=N\Nc2nc3ccccc3s2)Nc2ccccc21] | 69 | >60 | Positive binding | Specific but weak, 42D IC50 is ~15 μM |
| 7-96 | 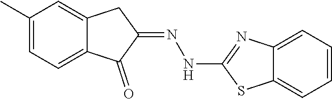 [SMILES: Cc1ccc2c(c1)C/C(=N/Nc1nc3ccccc3s1)C2=O] | 22 | 31 | inconclusive, needs to be repeated | Specific but weak, 42D IC50 is ~15 μM |
| 7-71 | 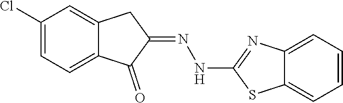 [SMILES: O=C1/C(=N\Nc2nc3ccccc3s2)Cc2cc(Cl)ccc21] | 35 | 47 | Positive binding | Specific to 42D but weak with IC50 ~15 μM |
| 7-72 | 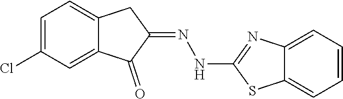 [SMILES: O=C1/C(=N\Nc2nc3ccccc3s2)Cc2ccc(Cl)cc21] | 8 | 17 | | Specific to 42D at low doses, affects 16D >15 μM |
| 7-73 | 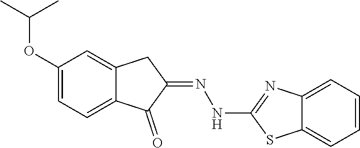 [SMILES: CC(C)Oc1ccc2c(c1)C/C(=N/Nc1nc3ccccc3s1)C2=O] | 19 | 26 | Positive binding | Specific to 42D at low doses (16D >15 μM) |
| 7-108 | 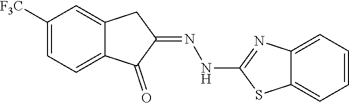 [SMILES: O=C1/C(=N\Nc2nc3ccccc3s2)Cc2cc(C(F)(F)F)ccc21] | 68 | >60 | Positive binding | Specific to 42D cells up to 10 μM (activity on 16D >15 μM) |
| 7-74 | 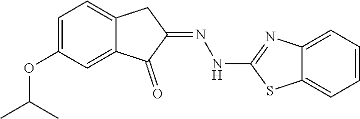 [SMILES: CC(C)Oc1ccc2c(c1)C(=O)/C(=N\Nc1nc3ccccc3s1)C2] | 6 | 17 | Positive binding | Specific to 42D at low doses, affects 16D >20 μM |
| 7-79 | 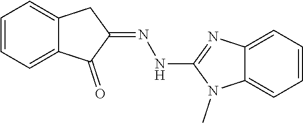 [SMILES: Cn1c(N/N=C2/Cc3ccccc3C2=O)nc2ccccc21] | 2 | 6 | | Specific but weak, 42D IC50 is >15 μM |
| 7-36 | 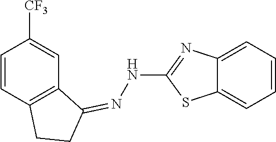 [SMILES: FC(F)(F)c1ccc2c(c1)/C(=N\Nc1nc3ccccc3s1)CC2] | 78 | >60 | DARTS- good binding | Specific to 42D, but weak activity |

In TABLES 1 and 2, in columns Met Stab % and T½, refers to compound's stability in MLM. There is a distinction in TABLES 1 and 2 difference between “Active” and “Specific” or “Specificity”. Some of the compounds were first tested to quantify inhibition of BRN2 transcriptional activity in a luciferase assay to determine BRN2 inhibition at 10 μM and 30 μM concentrations. Some compounds, were further tested for “Specificity” on two cell lines (i.e. 42D cells, which express BRN2 and 16D cells, which do not express any BRN2). Generally, compounds having low inhibitory activity were compounds with less than about ˜30% or activation at either concentration (i.e. 10 μM or 30 μM). Furthermore, some compounds that lacked specificity could also show off-target effects.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. The invention includes all embodiments and variations substantially as hereinbefore described and with reference to the examples and drawings.

1. A compound having the structure of Formula I or Formula II: 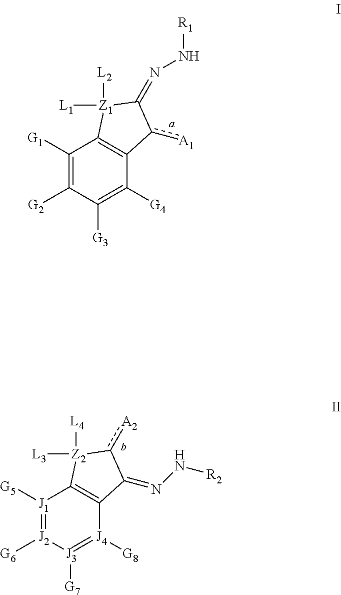
wherein, [symbol]
at a is either a double bond or two single bonds; [symbol]
at b is either a double bond or two single bonds;
Z₁ is C;
Z₂ is selected from C and 0;
L₁ is selected from H, CH₃, CH₂CH₃ and F;
L₂ is selected from H, CH₃, CH₂CH₃ and F;
L₃ is selected from H, CH₃, CH₂CH₃ and F or is absent when Z₂ is N;
L₄ is selected from H, CH₃, CH₂CH₃ and F;
A₁ is O when [symbol] at a is a double bond or A₁ is two H when [symbol] at a is two single bonds;
A₂ is O when [symbol] at b is a double bond or A₂ is two CH₃ groups or two H when [symbol] at b is two single bonds;
G₁ is selected from H, OH, CH₃, CF₃, 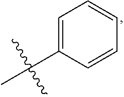 [SMILES: *c1ccccc1]
CN, OCH₃, OCHCH₃(CH₃), N(CH₂CH₃)CH₂CH₃, F, Cl and Br;
G₂ is selected from H, OH, CH₃, CF₃, 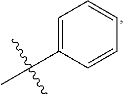 [SMILES: *c1ccccc1]
CN, OCH₃, OCHCH₃(CH₃), N(CH₂CH₃)CH₂CH₃, F, Cl and Br;
G₃ is selected from H, OH, CH₃, CF₃, 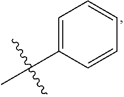 [SMILES: *c1ccccc1]
CN, OCH₃, OCHCH₃(CH₃), N(CH₂CH₃)CH₂CH₃, F, Cl and Br;
G₄ is selected from H, OH, CH₃, CF₃, 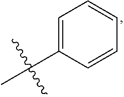 [SMILES: *c1ccccc1]
CN, OCH₃, OCHCH₃(CH₃), N(CH₂CH₃)CH₂CH₃, F, Cl and Br;
G₈ is selected from H, CH₃, OCH₃, CH₂CH₃, CF₃, F, Cl and Br or is absent when J₁ is N;
G₆ is selected from H, CH₃, OCH₃, CH₂CH₃, CF₃, F, Cl and Br or is absent when J₂ is N;
G₇ is selected from H, CH₃, OCH₃, CH₂CH₃, CF₃ and F or is absent when J₃ is N;
G₈ is selected from H, CH₃, OCH₃, CH₂CH₃ and F or is absent when J₄ is N;
J₁ is selected from C and N;
J₂ is selected from C and N;
J₃ is selected from C and N;
J₄ is selected from C and N;
R₁ is selected from 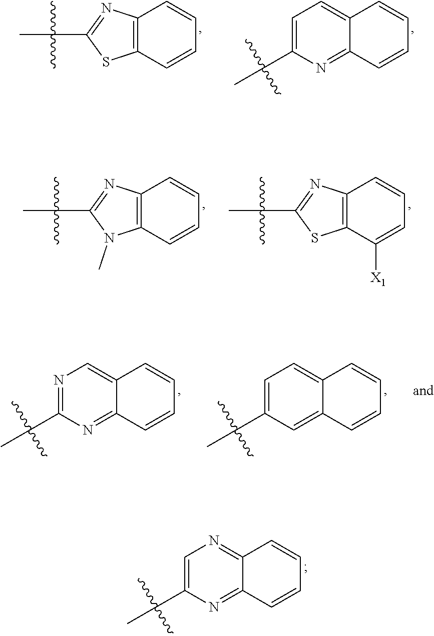
R₂ is selected from S and F 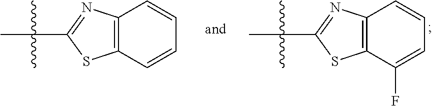 [SMILES: *c1nc2ccccc2s1 ; *c1nc2cccc(F)c2s1]
X₁ is selected from F, Cl and Br; and
provided that the compound is not

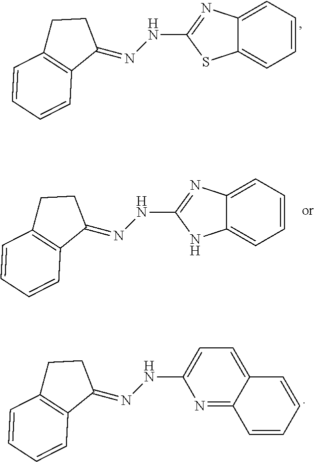 [SMILES: c1ccc2c(c1)CC/C2=N\Nc1nc2ccccc2s1 ; c1ccc2c(c1)CC/C2=N\Nc1nc2ccccc2[nH]1 ; c1ccc2c(c1)CC/C2=N\Nc1ccc2ccccc2n1]

2. The compound of claim 1, wherein
G₁ is selected from H, CH₃, CF₃, CN, OCH₃, F, Cl and Br;
G₂ is selected from H, OH, CH₃, CF₃, 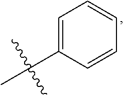 [SMILES: *c1ccccc1]
CN, OCH₃, OCHCH₃(CH₃), F, Cl and Br;
G₃ is selected from H, OH, CH₃, CF₃, 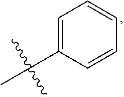 [SMILES: *c1ccccc1]
CN, OCH₃, OCHCH₃(CH₃), F, Cl and Br; and
G₄ is selected from H, CH₃, CF₃, CN, OCH₃, OCHCH₃(CH₃), F, Cl and Br; or
G₁ is selected from H, CF₃, F, Cl and Br;
G₂ is selected from H, CF₃, 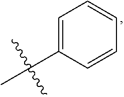 [SMILES: *c1ccccc1]
F, Cl and Br;
G₃ is selected from H, CF₃, CN, OCHCH(CH₃), F, Cl and Br; and
G₄ is selected from H, CF₃, CN, F, CI, and Br; or
G₅ is selected from H, CH₃, CF₃, F, Cl and Br;
G₆ is selected from H, CH₃, CF₃, F, Cl and Br;
G₇ is selected from H, CH₃, CF₃ and F or is absent when J₃ is N; and
G₈ is selected from H, CH₃ and F; or
G₅ is selected from H, F, Cl and Br;
G₆ is selected from H, F, Cl and Br;
G₇ is selected from H, CF₃ and F or is absent when J₃ is N; and
G₈ is selected from H, and F.

3.-5. (canceled)

6. The compound of claim 1, wherein
R₁ is selected from 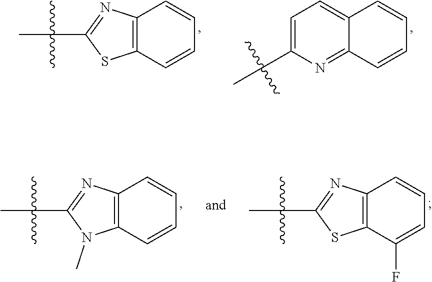 [SMILES: *c1nc2ccccc2s1 ; *c1ccc2ccccc2n1 ; *c1nc2ccccc2n1C ; *c1nc2cccc(F)c2s1]
and
R₂ is selected from

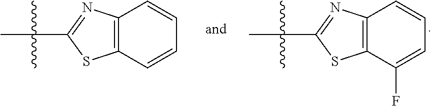 [SMILES: *c1nc2ccccc2s1 ; *c1nc2cccc(F)c2s1]

7. The compound of claim 1, wherein
R₁ is selected from 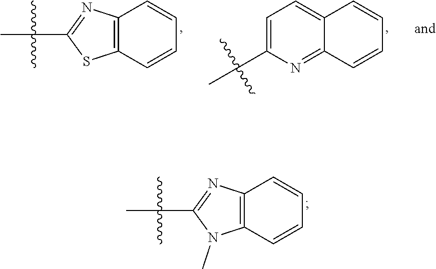 [SMILES: *c1nc2ccccc2s1 ; *c1ccc2ccccc2n1 ; *c1nc2ccccc2n1C]
and
R₂ is selected from

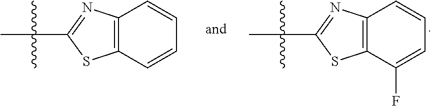 [SMILES: *c1nc2ccccc2s1 ; *c1nc2cccc(F)c2s1]

8. The compound of claim 1, wherein
Z₁ is C;
Z₂ is C;
L₁ is H;
L₂ is H;
L₃ is H; and
L₄ is H.

9. The compound of claim 1, wherein
A₁ is O when [symbol] at a is a double bond or A₁ is two H when [symbol] at a is two single bonds; and
A₂ is two H when [symbol] at b is two single bonds.

10. The compound of claim 1, wherein
J₁ is C;
J₂ is C;
J₃ is selected from C or N; and
J₄ is C.

11. The compound of claim 1, wherein the compound is selected from one or more of the following:

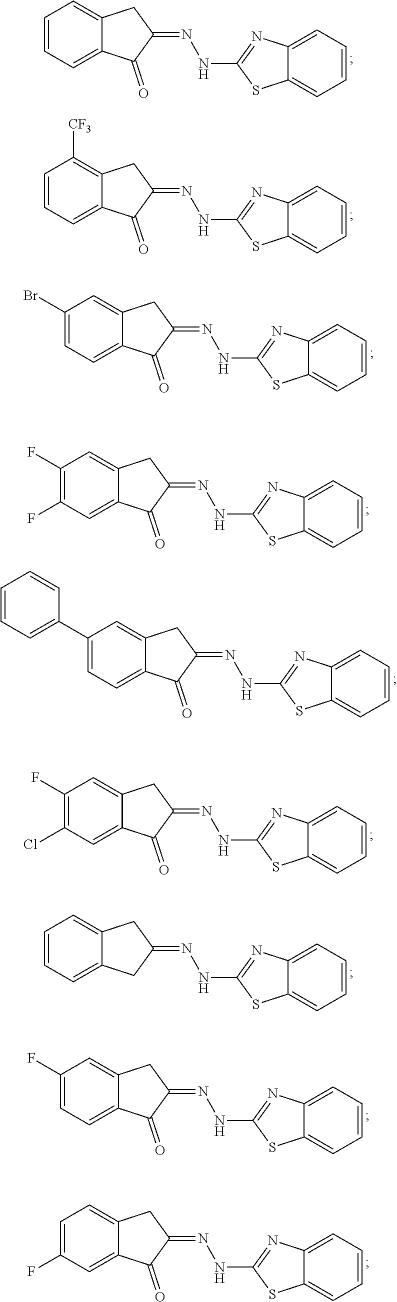

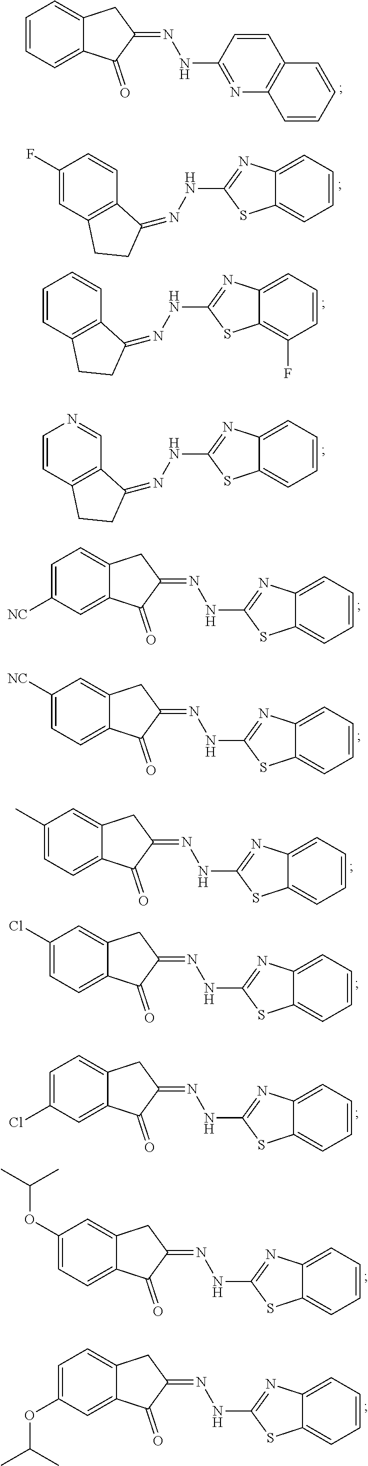

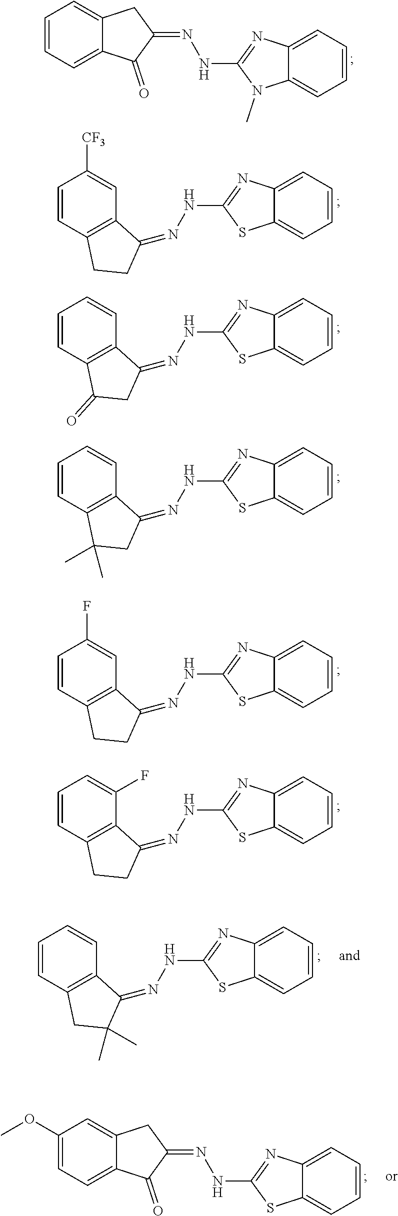

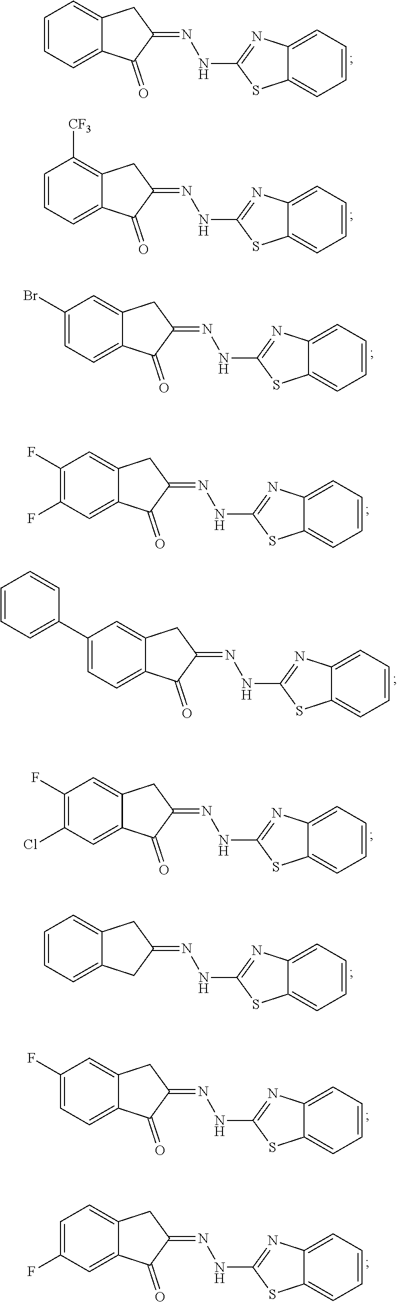

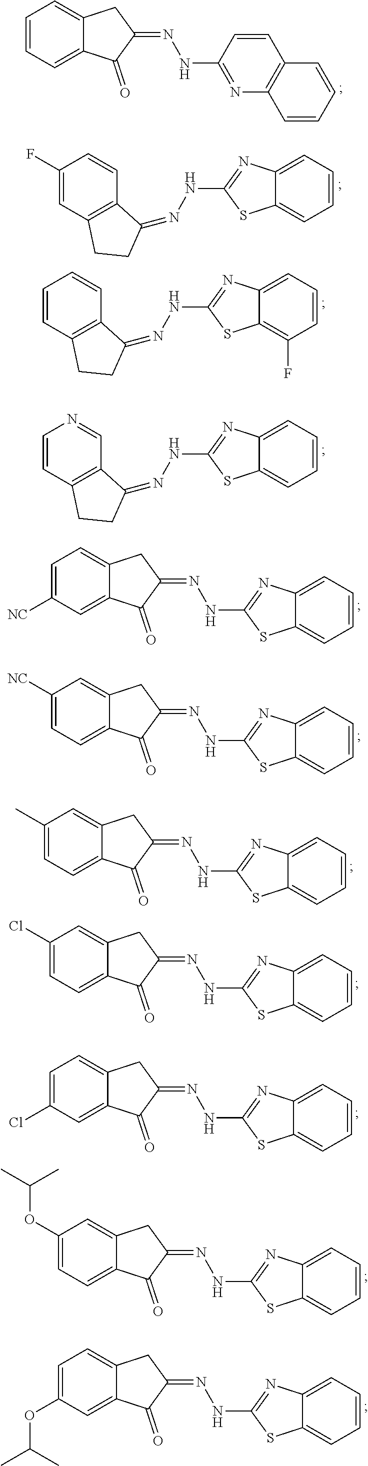

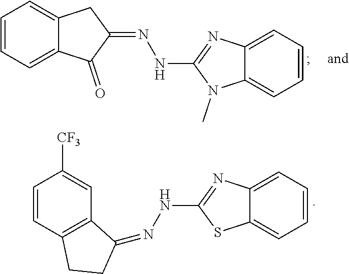 [SMILES: Cn1c(N/N=C2/Cc3ccccc3C2=O)nc2ccccc21 ; FC(F)(F)c1ccc2c(c1)/C(=N\Nc1nc3ccccc3s1)CC2]

12. (canceled)

13. The compound of claim 1, wherein the compound is selected from one or more of the following:

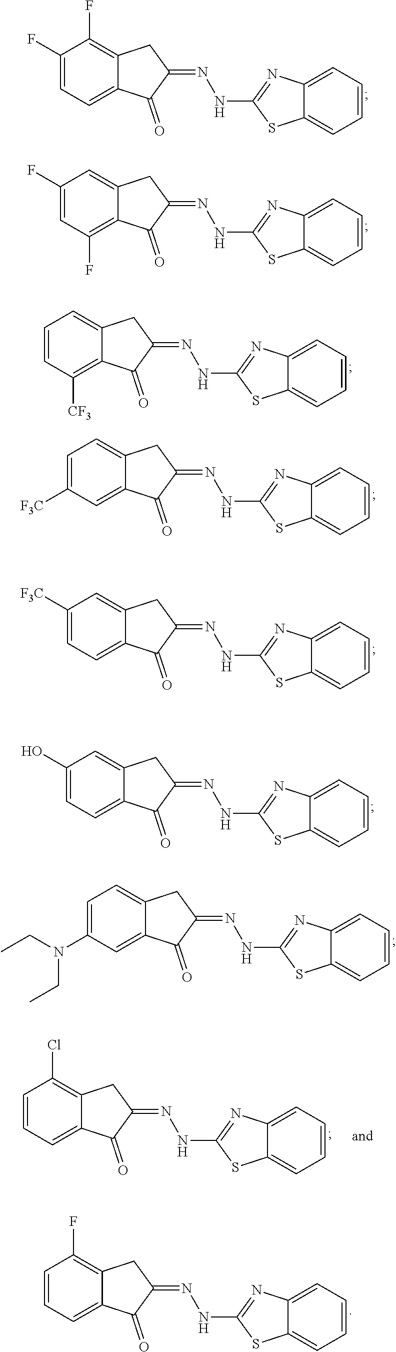

14. A method of inhibiting POU domain transcription factor BRN2, the method comprising administering a compound having the structure of Formula I or Formula II: 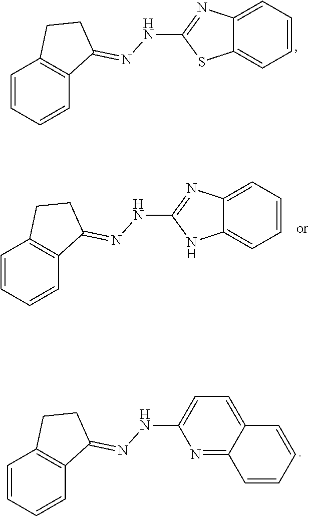 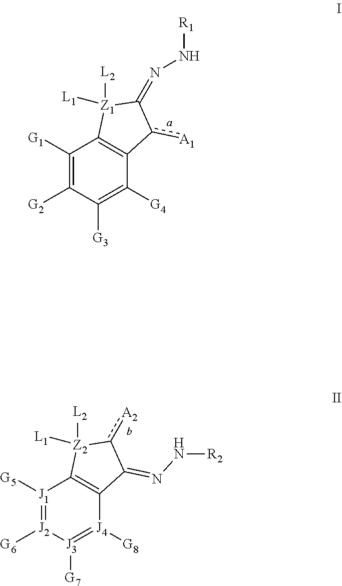
wherein, [symbol]
at a is either a double bond or two single bonds; [symbol]
at b is either a double bond or two single bonds;
Z₁ is C;
Z₂ is selected from C and 0;
L₁ is selected from H, CH₃, CH₂CH₃ and F;
L₂ is selected from H, CH₃, CH₂CH₃ and F;
L₃ is selected from H, CH₃, CH₂CH₃ and F or is absent when Z₂ is N;
L₄ is selected from H, CH₃, CH₂CH₃ and F;
A₁ is O when [symbol] at a is a double bond or A₁ is two H when [symbol] at a is two single bonds;
A₂ is O when [symbol] at b is a double bond or A₂ is two CH₃ groups or two H when [symbol] at b is two single bonds;
G₁ is selected from H, OH, CH₃, CF₃, 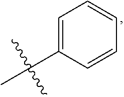 [SMILES: *c1ccccc1]
CN, OCH₃, OCHCH₃(CH₃), N(CH₂CH₃)CH₂CH₃, F, Cl and Br;
G₂ is selected from H, OH, CH₃, CF₃, 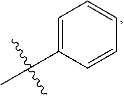 [SMILES: *c1ccccc1]
CN, OCH₃, OCHCH₃(CH₃), N(CH₂CH₃)CH₂CH₃, F, Cl and Br;
G₃ is selected from H, OH, CH₃, CF₃, 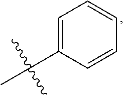 [SMILES: *c1ccccc1]
CN, OCH₃, OCHCH₃(CH₃), N(CH₂CH₃)CH₂CH₃, F, Cl and Br;
G₄ is selected from H, OH, CH₃, CF₃, 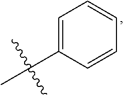 [SMILES: *c1ccccc1]
CN, OCH₃, OCHCH₃(CH₃), N(CH₂CH₃)CH₂CH₃, F, CI and Br;
G₅ is selected from H, CH₃, OCH₃, CH₂CH₃, CF₃, F, Cl and Br or is absent when J₁ is N;
G₆ is selected from H, CH₃, OCH₃, CH₂CH₃, CF₃, F, Cl and Br or is absent when J₂ is N;
G₇ is selected from H, CH₃, OCH₃, CH₂CH₃, CF₃ and F or is absent when J₃ is N;
G₈ is selected from H, CH₃, OCH₃, CH₂CH₃ and F or is absent when J₄ is N;
J₁ is selected from C and N;
J₂ is selected from C and N;
J₃ is selected from C and N;
J₄ is selected from C and N;
R₁ is selected from 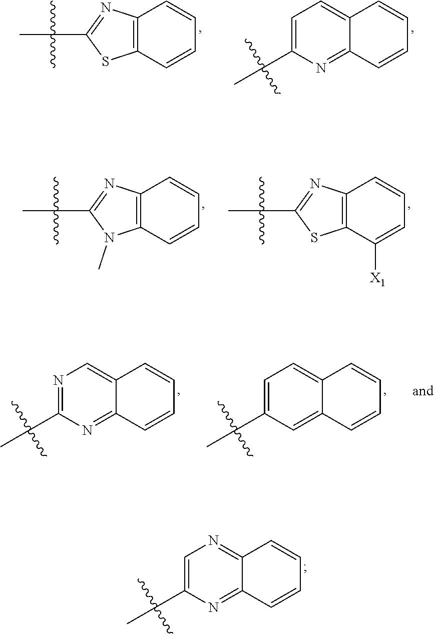
R₂ is selected from 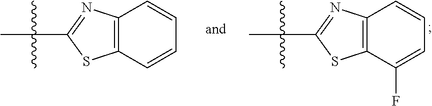 [SMILES: *c1nc2ccccc2s1 ; *c1nc2cccc(F)c2s1]
and
X₁ is selected from F, Cl and Br.

15. The method of claim 14, wherein the inhibiting of the POU domain transcription factor BRN2, is for the treatment of cancer.

16. The method of claim 15, wherein the cancer is a BRN2 expressing cancer.

17. The method of claim 15, wherein the cancer is selected from the following cancers: prostate cancer; lung cancer; bladder cancer; sarcoma; glioma; and melanoma.

18. The method of claim 17, wherein the prostate cancer is selected from: Neuroendocrine Prostate Cancer (NEPC); Prostate Adenocarcinoma; castration resistant prostate cancer (CRPC); androgen receptor pathway inhibitor (ARPI) resistant prostate cancer; enzalutamide (ENZ)-resistant (ENZ^{R}); and Abiraterone (Abi)-resistant (ABI_{R}).

19. The method of claim 17, wherein the lung cancer is small cell lung cancer (SCLC) or lung adenocarcinoma.

20. The method of claim 17, wherein the bladder cancer is small cell bladder cancer (SCBC).

21. The method of claim 17, wherein the sarcoma is Ewing's sarcoma.

22. The method of claim 17, wherein the glioma is glioblastoma multiforme.

23. The method of claim 16, wherein the BRN2 expressing cancer is selected from the following: bladder cancer; cholangiocarcinoma; colorectal cancer; diffuse large B-cell lymphoma (DLBC); liver cancer; ovarian cancer; thymoma; thyroid cancer; clear cell renal cell carcinoma (CCRCC); chromophobe renal cell carcinoma (ChRCC); prostate cancer; breast cancer; uterine cancer; pancreatic cancer; cervical cancer; uveal melanoma; acute myeloid leukemia (AML); head and neck cancer; small cell lung cancer (SCLC); lung adenocarcinoma sarcoma; mesothelioma; adenoid cystic carcinoma (ACC); sarcoma; testicular germ cell cancer; uterine cancer; pheochromocytoma and paraganglioma (PCPG); melanoma; glioma; glioblastoma multiforme; T-cell Acute Lymphoblastic Leukemia; T-cell Lymphoma, medulloblastoma; and neuroblastoma.

24.-41. (canceled)
